(19) United States
(12) Patent Application Publication
(10) Pub. No.: US 2011/0171645 A1
(43) Pub. Date: Jul. 14, 2011

(54) METHODS OF DIAGNOSING ACUTE CARDIAC ALLOGRAFT REJECTION

(75) Inventors: Bruce McManus, Vancouver (CA); Zsuzsanna Hollander, Vancouver (CA); Alice Mui, Burnaby (CA); Robert Balshaw, Vancouver (CA); Robert Mcmaster, Vancouver (CA); Paul Keown, Delta, CA (US); Gabriela Cohen Freue, Vancouver (CA); Pooran Qasimi, Surrey, CA (US); Raymond Ng, Vancouver (CA); David Lin, Richmond (CA); David Wishart, Edmonton (CA); Axel Bergman, Vancouver (CA)

(73) Assignee: The University of British Columbia, Vancouver (CA)

(21) Appl. No.: 12/937,220

(22) PCT Filed: Apr. 9, 2009
(86) PCT No.: PCT/CA09/00516

§ 371 Date: Mar. 9, 2011

Related U.S. Application Data
(60) Provisional application No. 61/071,038, filed on Apr. 9, 2008.
(60) Provisional application No. 61/071,037, filed on Apr. 9, 2008.
(60) Provisional application No. 61/071,057, filed on Apr. 10, 2008.
(60) Provisional application No. 61/157,161, filed on Mar. 3, 2009.

(51) Int. Cl. C12Q 1/68; G01N 33/53; H01J 49/26

(52) U.S. Cl. 435/6.11; 436/501; 435/7.92; 250/282

(57) ABSTRACT

The present invention relates to methods of diagnosing acute rejection of a cardiac allograft using genomic expression profiling, proteomic expression profiling, metabolite profiling, or alloreactive T-cell genomic expression profiling,

This application claims priority benefit of U.S. Provisional applications 61/071,038, filed Apr. 9, 2008; U.S. /071,037, filed Apr. 9, 2008; U.S. 61/071,07 filed Apr. 10, 2008; and U.S. 61/157,161, filed Mar. 3, 2009, all of which are herein incorporated by reference.

### FIELD OF INVENTION

The present invention relates to methods of diagnosing acute rejection of a cardiac allograft using genomic expression profiling, proteomic expression profiling, metabolite profiling, or alloreactive T-cell genomic expression profiling.

### BACKGROUND OF THE INVENTION

Transplantation is considered the primary therapy for patients with end-stage vital organ failure. While the availability of immunosuppressants such as cyclosporine and Tacrolimus has improved allograft recipient survival and wellbeing, identification of rejection of the allograft as early and as accurately as possible, and effective monitoring and adjusting immunosuppressive medication doses is still of primary importance to the continuing survival of the allograft recipient.

Rejection of an allograft may be generally described as the result of recipient's immune response to nonself antigens expressed by the donor tissues. Acute rejection may occur within days or weeks of the transplant, while chronic rejection may be a slower process, occurring months or years following the transplant.

At present, invasive biopsies, such as endomyocardial, liver core, and renal fine-needle aspiration biopsies, are widely regarded as the gold standard for the surveillance and diagnosis of allograft rejections, but are invasive procedures which carry risks of their own (e.g. Mehra M R, et al. Curr. Opin. Cardiol. 2002 March; 17(2):131-136.). Biopsy results may also be subject to reproducibility and interpretation issues due to sampling errors and inter-observer variabilities, despite the availability of international guidelines such as the Banff schema for grading liver allograft rejection (Ormonde et al 1999. Liver Transplantation 5:261-268) or the Revised ISHLT transplantation scale (Stewart et al. 2005. J Heart Lung Transplant, 2005; 24: 1710-20). Although less invasive (imaging) techniques have been developed such as angiography and IVUS for monitoring chronic heart rejection, these alternatives are also susceptible to limitations similar to those associated with biopsies.

The severity of allograft rejection as determined by biopsy may be graded to provide standardized reference indicia. The International Society for Heart and Lung Transplantation scale (ISHLT) provides a means of grading biopsy samples for heart transplant subjects (Table 1).

TABLE 1
International Society for Heart and Lung Transplantation grading
of heart transplant rejection for histopathologic biopsy analysis

| Grade | Comment |
|---|---|
| 0R | No acute cellular rejection: No evidence of mononuclear |
| | inflammation or myocyte damage or necrosis. |
| 1R | Mild, low-grade, acute cellular rejection: Mononuclear cells are |
| | present and there may be limited myocyte damage and necrosis. |
| 2R | Moderate, intermediate-grade, acute cellular rejection: Two or |
| | more foci of mononuclear cells with associated myocyte damage |
| | and necrosis are present. The damage may be found in the same |
| | biopsy, or two separate biopsies. Eosinophils may be present. |
| 3R | Severe, high-grade, acute cellular rejection: Widespread, diffuse |
| | myocyte damage and necrosis, and disruption of normal archi- |
| | tecture across several biopsies. Edema, interstitial hemorrhage |
| | and vasculitis may be present. The infiltrate may be |
| | polymorphous. |

Indicators of allograft rejection may include a heightened and localized immune response as indicated by one or more of localized or systemic inflammation, tissue injury, allograft infiltration of immune cells, altered composition and concentration of tissue- and blood-derived proteins, differential oxygenation of allograft tissue, edema, thickening of the endothelium, increased collagen content, altered intramyocardial blood flow, infection, necrosis of the allograft and/or surrounding tissue, and the like.

Allograft rejection may be described as ‘acute’ or ‘chronic’. Acute rejection is generally considered to be rejection of a tissue or organ allograft within ˜6 months of the subject receiving the allograft. Acute rejection may be characterized by cellular and humoral insults on the donor tissue, leading to rapid graft dysfunction and failure of the tissue or organ. Chronic rejection is generally considered to be reject of a tissue or organ allograft beyond 6 months, and may be several years after receiving the allograft. Chronic rejection may be characterized by progressive tissue remodeling triggered by the alloimmune response may lead to gradual neointimal formation within arteries, contributing to obliterative vasculopathy, parenchymal fibrosis and consequently, failure and loss of the graft. Depending on the nature and severity of the rejection, there may be overlap in the indicators or clinical variables observed in a subject undergoing, or suspected of undergoing, allograft rejection—either chronic or acute.

Attempts have been made to reduce the number of biopsies per patient, but may be generally unsuccessful, due in part to the difficulty in pinpointing the sites where rejection starts or progresses, and also to the difficulty in assessing tissue without performing the actual biopsy. Noninvasive surveillance techniques have been investigated, and may provide a reasonable negative prediction of allograft rejection, but may be of less practical utility in a clinical setting (Mehra et al., supra).

The scientific and patent literature is blessed with reports of this marker or that being important for identification/diagnosis/prediction/treatment of every medical condition that can be named. Even within the field of allograft rejection, a myriad of markers are recited (frequently singly), and conflicting results may be presented. This conflict in the literature, added to the complexity of the genome (estimates range upwards of 30,000 transcriptional units), the variety of cell types (estimates range upwards of 200), organs and tissues, and expressed proteins or polypeptides (estimates range upwards of 80,000) in the human body, renders the number of possible nucleic acid sequences, genes, proteins, metabolites or combinations thereof useful for diagnosing acute organ rejection is staggering. Variation between individuals presents additional obstacles, as well as the dynamic range of protein concentration in plasma (ranging from 10⁻⁶ to 10³ μg/mL) with many of the proteins of potential interest existing at very low concentrations) and the overwhelming quantities of the few, most abundant plasma proteins (constituting ˜99% of the total protein mass.

The CARGO study (Cardiac Allograft Rejection Gene Expression Observation) (Deng et al., 2006. Am J. Transplantation 6:150-160) used custom microarray analysis of ˜7300 genes and RT-PCR to examine gene expression profile in subjects exhibiting an ISHLT score of 3 A or greater in samples taken 6 months or more post-transplant.

Metabolite profiling has been suggested as a tool for assessing organ function, disease states and the like (Wishart 2005. 5:2814-2820). Numerous publications are found relating generally to this field, and recently a database of the human ‘metabolome’ has been published (Wishart et al, 2007. Nucleic Acids Research 35:D521-D526), however identification of particular metabolite profiles or signatures useful in assessing or diagnosing allograft rejection remains to be determined.

Immune cells that have a role in recognizing may be useful as indicators of allograft rejection. WO 2005/05721 describes methods for distinguishing immunoreactive T-lymphocytes that bind specifically to donor antigen presenting cells, providing a population of T-lymphocytes that are specifically immunoreactive to the donor antigens. Again however, particular markers that may be useful in assessing or diagnosing allograft rejection remain to be determined.

Traum et al., 2005 (Pediatr. Transplant 9(6):700-711) provides a general overview of transplantation proteomics. Exploration of biomarkers directly in the plasma proteome presents two main challenges—the dynamic range of protein concentrations extends from 10⁻⁶ to 10³ μg/mL (Anderson et al. 2004. Mol Cell Proteomics 3:311-326), with many of the proteins of potential interest existing at very low concentrations and the most abundant plasma proteins comprising as much as 99% of the total protein mass.

Maintenance or measurement of B2M serum levels in heart transplant patients was suggested as helpful in managing long-term immunosuppressive therapy (Erez et al., 1998. J Heart Lung Transplant 17:538-541). PCT Publication WO 2009/003142 disclose that B2M, along with another protein may be useful as biomarkers for peripheral artery disease.

Borozdenkova et al. 2004 (J. Proteome Research 3:282-288) discloses that alpha B-crystallin and tropmyosin were elevated in a set of cardiac transplant subjects.

Ishihara, 2008 (J. Mol Cell Cardiology 45:S33) discloses that ADIPOQ may have a role in cardiac transplantation, and Nakano (Transplant Immunology 2007 17:130-136) suggests that upregulation of ADIPOQ may be necessary for overcoming rejection in liver transplant subjects.

Antibodies that bind SHBG (PCT Publication WO 2007/024715) and F10 (PCT Publication WO 2005/020927) are suggested as being useful in preventing graft rejection.

SERPINF1 and C1Q are disclosed as biomarkers associated with an increased risk of a cardiovascular event; the biomarkers may be detected in a sample of an atherosclerotic plaque from a subject (PCT Publication WO 2009/017405); sequences for SERPINF1 may also be useful in an assay to select optimal blood vessel graft (US Publication 2006/0003338).

Complement is also known to have a role in rejection of allografts—Csencits et al., 2008 (Am J. Transplantation 8:1622-1630) summarizes past studies on various complement components and observes an accelerated humoral immune response in C1Q−/− mice allograft recipients.

PCT Publications WO2006/083986, WO206/122407, US Publications 2008/0153092, 2006/0141493 and U.S. Pat. No. 7,235,358 disclose methods for using panels of biomarkers (proteomic or genomic) for diagnosing or detecting various disease states ranging from cancer to organ transplantation

Alakulppi et al, 2007 (Transplantation 83:791-798) discloses the diagnosis of acute renal allograft rejection using RT-PCR for eight markers.

A review by Fildes et al 2008 (Transplant Immunology 19:1-11) discusses the role of cell types in immune processes following lung transplantation, and discloses that AICL (CLEC2B) interaction with NK cell proteins may have a role in acute and chronic rejection

Integration of multiple platforms (proteomics, genomics) has been suggested for diagnosis and monitoring of various cancers, however discordance between protein and mRNA expression is identified in the field (Chen et al., 2002. Mol Cell Proteomicsl:304-313; Nishizuka et al., 2003 Cancer Research 63:5243-5250). Previous studies have reported low correlations between genomic and proteomic data (Gygi S P et al. 1999. Mol Cell Biol. 19:1720-1730; Huber et al., 2004 Mol Cell Proteomics 3:43-55).

Methods of assessing or diagnosing allograft rejection that are less invasive, repeatable and more robust (less susceptible to sampling and interpretation errors) are greatly desirable.

### SUMMARY OF THE INVENTION

The present invention relates to methods of diagnosing acute rejection of a cardiac allograft using one or more of genomic expression profiling, proteomic expression profiling, metabolite profiling, or alloreactive T-cell genomic expression profiling,

The complex pathobiology of acute cardiac allograft rejection is reflected in the heterogeneity of markers identified herein. Markers identified herein distribute over a range of biological processes: cellular and humoral immune responses, acute phase inflammatory pathways, matrix remodeling effects, lipid metabolism, stress response and the like.

In accordance with one aspect of the invention, there is provided a method of diagnosing acute allograft rejection in a subject using genomic expression profiling, the method comprising: a) determining the expression profile of one or more than one genomic markers in a biological sample from the subject, the markers selected from the group comprising TRF2, SRGAP2P1, KLF4, YLPM1, BID, MARCKS, CLEC2B, ARHGEF7, LYPLAL1, WRB, FGFR1OP2, MBD4; b) comparing the expression profile of the one or more than one markers to a control profile; and c) determining whether the expression level of the one or more than one genomic markers is increased or decreased relative to the control profile, wherein increase or decrease of the at least nine markers is indicative of the acute rejection status.

In accordance with another aspect of the invention, the method further comprises obtaining a value for one or more clinical variables and comparing the one or more clinical variables to a control.

In accordance with another aspect of the invention, the method may further comprise determining the genomic expression profile of one or more markers listed in Table 6.

In accordance with another aspect of the invention, TRF2 and FGFR1OP2 may be increased relative to a control, and SRGAP2P1, KLF4, YLPM1, BID, MARCKS, CLEC2B, ARHGEF7, LYPLAL1, WRB, MBD4 may be decreased relative to a control.

In accordance with another aspect of the invention, the control is a non-rejection, allograft recipient subject or a non-allograft recipient subject.

In accordance with another aspect of the invention, the control is an autologous control.

In accordance with another aspect of the invention, there is provided a kit for assessing, predicting or diagnosing acute allograft rejection in a subject using genomic expression profiling, the kit comprising reagents for specific and quantitative detection of one or more than one of TRF2, SRGAP2P1, KLF4, YLPM1, BID, MARCKS, CLEC2B, ARHGEF7, LYPLAL1, WRB, FGFR1OP2, MBD4, along with instructions for the use of such reagents and methods for analyzing the resulting data. The kit may further comprise one or more oligonucleotides for selective hybridization to one or more than one gene or transcript encoding TRF2, SRGAP2P1, KLF4, YLPM1, BID, MARCKS, CLEC2B, ARHGEF7, LYPLAL1, WRB, FGFR1OP2, MBD4. Instructions or other information useful to combine the kit results with those of other assays to provide a non-rejection cutoff index or control for the diagnosis of a subject's rejection status may also be provided in the kit.

In accordance with one aspect of the invention, there is provided a method of diagnosing acute allograft rejection in a subject, the method comprising: a) determining the expression profile of five or more than five markers in a biological sample from the subject, the markers selected from the group comprising a polypeptide encoded by B2M, F10, CP, CST3, ECMP1, CFH, C1QC, CFI, APCS, C1R, SERPINF1, PLTP, ADIPOQ and SHBG; b) comparing the expression profile of the one or more than one markers to a control profile; and c) determining whether the expression level of the one or more than one markers is increased or decreased relative to the control profile, wherein increase or decrease of the one or more than one markers is indicative of the acute rejection status.

In accordance with another aspect of the invention, the five or more than five markers include PLTP, ADIPOQ, B2M, F10 and CP.

In accordance with another aspect of the invention, the five or more than five markers include PLTP, ADIPOQ, B2M, F10 and CP, and one or more than one of ECMP1, C1QC, C1R and SERPINF1.

In accordance with another aspect of the invention, the method further comprises obtaining a value for one or more clinical variables and comparing the one or more clinical variables to a control.

In accordance with another aspect of the invention, B2M, F10, CP, CST3, ECMP1, CFH, C1QC, CFI, APCS, C1R and/or SERPINF1 may be increased relative to a control, and PLTP, ADIPOQ and/or SHBG may be decreased relative to a control.

In accordance with another aspect of the invention, the control is a non-rejection, allograft recipient subject or a non-allograft recipient subject

In accordance with another aspect of the invention, the control is an autologous control.

In accordance with another aspect of the invention, there is provided a kit for assessing, predicting or diagnosing acute allograft rejection in a subject, the kit comprising reagents for specific and quantitative detection of five or more than five of comprising a polypeptide encoded by B2M, F10, CP, CST3, ECMP1, CFH, C1QC, CFI, APCS, C1R, SERPINF1, PLTP, ADIPOQ and SHBG, along with instructions for the use of such reagents and methods for analyzing the resulting data. Instructions or other information useful to combine the kit results with those of other assays to provide a non-rejection cutoff index or control for the diagnosis of a subject's rejection status may also be provided in the kit.

In accordance with another aspect of the invention, the five or more than five markers include a polypeptide encoded by PLTP, ADIPOQ, B2M, F10 and CP.

In accordance with another aspect of the invention, the five or more than five markers include PLTP, ADIPOQ, B2M, F10 and CP, and one or more than one of ECMP1, C1QC, C1R and SERPINF1.

In accordance with one aspect of the invention, there is provided a method of diagnosing acute allograft rejection in a subject, the method comprising: a) determining the expression profile of one or more than one markers in a biological sample comprising alloreactive T-cells from the subject, the one or more than one markers selected from the group comprising KLF12, TTLL5, 239901_at, 241732_at, OFD1, MIRH1, WDR21A, EFCAB2, TNRC15, LENG10, MYSM1, 237060_at, C19orf59, MCL1, ANKRD25, MYL4; b) comparing the expression profile of the one or more than one markers to a non-rejector alloreactive T-cell control profile; and c) determining whether the expression level of the markers is increased or decreased relative to the control profile, wherein up-regulation or down-regulation of the markers is indicative of the acute rejection status.

In accordance with another aspect of the invention, KLF12, TTLL5, 239901_at, 241732_at, OFD1, MIRH1, WDR21A, EFCAB2, TNRC15, LENG10 and MYSM1 may be decreased relative to a control, and 237060_at, C19orf59, MCL1, ANKRD25 and MYL4 may be increased relative to a control.

In accordance with another aspect of the invention, there is provided a kit for diagnosing acute allograft rejection in a subject, the kit comprising reagents for isolation of alloreactive T-cells, reagents for specific and quantitative detection of KLF12, TTLL5, 239901_at, 241732_at, OFD1, MIRH1, WDR21A, EFCAB2, TNRC15, LENG10, MYSM1, 237060_at, C19orf59, MCL1, ANKRD25, MYL4, along with instructions for the use of such reagents and methods for analyzing the resulting data. The kit may further comprise one or more oligonucleotides for selective hybridization to one or more than one of a gene or transcript encoding some or part of KLF12, TTLL5, 239901_at, 241732_at, OFD1, MIRH1, WDR21A, EFCAB2, TNRC15, LENG10, MYSM1, 237060_at, C19orf59, MCL1, ANKRD25, MYL4. Instructions or other information useful to combine the kit results with those of other assays to provide a non-rejection cutoff index or control for the diagnosis of a subject's rejection status may also be provided in the kit.

In accordance with one aspect of the invention, there is provided a method of diagnosing acute allograft rejection in a subject, the method comprising: a) determining the expression profile of one or more than one markers in a biological sample from the subject, the one or more than one markers selected from the group comprising KLF12, TTLL5, 239901_at, 241732_at, OFD1, MIRH1, WDR21A, EFCAB2, TNRC15, LENG10, MYSM1, 237060_at, C19orf59, MCL1, ANKRD25, MYL4; b) comparing the expression profile of the one or more than one markers to a control profile; and c) determining whether the expression level of the markers is increased or decreased relative to the control profile, wherein increase or decrease of the markers is indicative of the acute rejection status.

In accordance with another aspect of the invention, the method further comprises obtaining a value for one or more clinical variables and comparing the one or more clinical variables to a control.

In accordance with another aspect of the invention, the control is a non-rejection, allograft recipient subject or a non-allograft recipient subject.

In accordance with another aspect of the invention, the control is an autologous control.

In accordance with another aspect of the invention, there is provided a method of diagnosing cardiac allograft rejection using a metabolite profile in a subject, the method comprising the following steps: measuring the concentration of at least three markers in a biological sample from the subject, the markers selected from the group comprising creatine, taurine, serine, carnitine and glycine; comparing the concentration of each of the at least three markers to a non-rejector metabolite profile cutoff index, and determining a rejection status of the subject; whereby the rejection status of the subject is indicated by the concentration of each of the at least three markers being above or below the control metabolite profile cutoff index.

In accordance with another aspect of the invention, at least three markers are taurine, serine and glycine, the concentration of the markers is an absolute comparison, and each of taurine, serine and glycine markers are decreased relative to a non-rejection metabolite cutoff index.

In accordance with another aspect of the invention, the at least three markers are glycine, creatine and carnitine; the concentration of the markers is relative to a metabolite baseline comparison; and each of creatine and carnitine markers are increased relative to a non-rejection metabolite profile cutoff index, and glycine marker is decreased relative to a non-rejection metabolite profile cutoff index.

In accordance with another aspect of the invention, the method of diagnosing cardiac allograft rejection using a metabolite profile further comprises obtaining a value for one or more clinical variables.

It is therefore an advantage of some aspects of the present invention to provide a method of diagnosing acute allograft rejection without a biopsy of the transplanted tissue or organ.

This summary of the invention does not necessarily describe all features of the invention. Other aspects, features and advantages of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows a sample map of the subject in the study. Squares indicate the time points for which a sample for microarray data was available. Circles designate diagnosis of a related tissue biopsy with ≧2R rejection versus the triangles which illustrate 1R rejection in the related tissue biopsy. Xs are the samples linked to a tissue biopsy with no rejection.

FIG. 2 shows the results of subject classification using a biomarker panel of 12 genes. Subjects were previously determined to have acute rejection (≧2R) or no rejection (0R). The list of genes for this biomarker panel include: Transferrin receptor 2 (TFR2), SLIT-ROBO Rho GTPase activating protein 2 Pseudogene 1 (SRGAP2P1), Kruppel-like factor 4 (KLF4), YLP motif containing 1 (YLPM1), BH3 interacting domain death agonist (BID), Myristoylated alanine-rich protein kinase C substrate (MARCKS), C-type lectin domain family 2, member B (CLEC2B), Rho guanine nucleotide exchange factor (GEF) 7, (ARHGEF7/BETA-PIX), Lysophospholipase-like 1 (LYPLAL1), Tryptophan rich basic protein (WRB), FGFR1 oncogene partner 2 (FGR1OP2), Methyl-CpG binding domain protein 4 (MBD4). Diamond—acute rejector (AR); Circle—non rejector (NR)

FIG. 3 shows a proposed relationship between the biomarkers ARHGEF7, TRF2, BID, MARCKS, KLF4, CLEC2B and MBD4.

FIG. 4 shows a summary of subject classification using clinical variable profiling. Diamond—acute rejector (AR); Circle—non rejector (NR)

FIG. 5. Proportion of protein group codes (PGC's) identified using different peptide counts (p). Average peptide counts across iTRAQ runs were used for PGC's identified in multiple runs. “Total” (horizontal slash bar), “Analyzed” (diagonal slash bar) and “Panel” (vertical slash bar) represent the sets of PGC's detected in at least one of the 18 samples included in the discovery, detected in at least ⅔ of the AR (acute rejection) and NR (non-rejection) groups, and identified with significant differential relative concentrations, respectively.

FIG. 6. Plasma protein panel A proteomic markers. A. Average of the score generated by LDA based on panel A for all available AR samples (solid line) and NR samples (dashed or stippled line) at each timepoint. B. Score when patients transitioned between NR and AR episodes. The first consecutive AR time points were considered and averaged (AR) from AR patients (solid line). Consecutive timepoints of NR before (NR before AR) and after (NR after AR) AR were considered and averaged from the same patients. A control curve (dashed or stippled line) was constructed for NR patients matched as closely as possible to AR patients by available timepoints. Standard deviations within each group are represented using vertical bars.

FIG. 7: Internal validation of proteomic markers. Classification of 13 new subject samples using panel A (FDR<25%) and panel B (selected by SDA). Scores generated by both classifiers were re-centered to set both the cut-off lines for classification at zero. Average scores for each AR (open star) and NR (solid star) samples in the training set are displayed using red and black asterisks, respectively. Scores for each AR (solid triangle) and NR (solid square) samples in the test set are shown. Samples with positive values were classified as AR and those with negative values were classified as NR by LDA.

FIG. 8: Technical validation of proteomic markers. iTRAQ versus ELISA relative protein levels (relative to pooled control) of 5 validated proteins from the 18 subject samples used in the discovery. AR samples=open circles; NR samples=solid circle. Spearman's correlation coefficients (Cor) and p-values from a test of positive correlation are displayed for each protein in the bottom-right of each plot.

FIG. 9 shows a sample map of the subjects whose samples were included in the metabolomics study. Square indicates the time points for which a sample for metabolomic data was available. Circle indicates diagnosis of a related tissue biopsy with ≧2R rejection versus the triangles which illustrate 1R rejection in the related tissue biopsy. X are the samples linked to a tissue biopsy with no rejection.

FIG. 10 shows the grouping of subjects in metabolomics study, exhibiting 0R or >2R rejection of a cardiac allograft when metabolite concentrations were analyzed using a moderated t-test. When the absolution concentration of the post-transplant sample was analyzed, three metabolites were statistically significant using a moderated t-test. The horizontal line illustrates the mean of each group. The total sample population included six samples from acute rejector (AR) subjects and 21 from non-rejector (NR) subjects. Diamond—acute rejector (AR); Circle—non rejector (NR)

FIG. 11 shows the grouping of subjects exhibiting 0R or >2R rejection when metabolite concentrations were analyzed using a moderated t-test. When the concentration of the post-transplant sample was compared to the baseline concentration, three metabolites were statistically significant using a moderated t-test. The line illustrates the mean of each group. The total sample population included six samples from AR subjects and 21 from NR subjects. Diamond—acute rejector (AR); Circle—non rejector (NR)

FIG. 12 shows a sample map of the subjects in the alloreactive T-cell subject population. Squares indicate the time points for which a sample for microarray data was available. Circles designate diagnosis of a related tissue biopsy with ≧2R rejection versus the triangles which illustrate 1R rejection in the related tissue biopsy. Xs are the samples linked to a tissue biopsy with no rejection.

FIG. 13: Alloreactive T cell gene biomarkers enhance the classification ability of whole blood gene biomarkers to discriminate acute from no rejection. A panel of genes from whole blood are used as a biomarker panel (A) to differentiate acute from no rejection. When 2 genes from the Alloreactive T cell list are added, the classification is even more separated (B). Diamond—acute rejector (AR); Circle—non rejector (NR)

FIG. 14 shows examples of Protein Coverage Maps for proteins in panels A and B (Table 10) for iTRAQ experiment (this run was used to process B-314-W12, B-314-W6 and B-415-W12. Proteins in each group (with a common Protein Group Code, PGC) are shown, and aligned where two or more proteins share a PGC. Double underline, no bold=peptides identified with a confidence interval (confidence of identification)≧95%; Single underline, no bold=50%≦CI<95%; No underline, bold=0%≦CI<50%; and Plain text (no underline, no bold) for no detected peptides. A: PGC 151: Phospholipid transfer protein precursor—IPI00643034.2 (PLTP) Isoform 1 of Phospholipid transfer protein precursor (SEQ ID NO: 1); IPI00217778.1 (PLTP) Isoform 2 of Phospholipid transfer protein precursor (SEQ ID NO: 2); IPI00022733.3 (PLTP) 45 kDa protein (SEQ ID NO: 3). B: B: PGC 92: Adiponectin precursor IPI00020019.1 (SEQ ID NO: 4). C: PGC 61: Pigment epithelium-derived factor precursor IPI00006114.4 (SEQ ID NO: 14). D: PGC 188: Beta-2-microglobulin—IPI00868938.1 (−) Beta-2-microglobulin (SEQ ID NO: 5); IPI00796379.1 (B2M) B2M protein (SEQ ID NO: 6); IPI00004656.2 (B2M) Beta-2-microglobulin (SEQ ID NO: 7). E: PGC 84: Coagulation factor X precursor IPI00019576.1 (SEQ ID NO: 8). F: PGC 6: Ceruloplasmin (IPI00017601.1 (SEQ ID NO: 9). G: PGC 76: Complement C1q subcomponent subunit C precursor IPI00022394.2 (SEQ ID NO: 12). H: PGC 26: Complement C1r subcomponent precursor IPI00296165.5 (SEQ ID NO: 13). I: PGC 62: Extracellular matrix protein—IPI00645849.1 Extracellular matrix protein 1 (SEQ ID NO: 10); IPI00003351.2 Extracellular matrix protein 1 precursor (SEQ ID NO: 11). Peptides that were identified in the iTRAQ experiments are listed in FIG. 17.

FIG. 15 shows examples of Protein CoverageMaps for additional identified proteomic markers (Table 10) for iTRAQ experiment (this run was used to process B-314-W12, B-314-W6 and B-415-W12. Proteins in each group (with a common Protein Group Code, PGC) are shown, and aligned where two or more proteins share a PGC. Double underline, no bold=peptides identified with a confidence interval (confidence of identification)≧95%; Single underline, no bold=50%≦CI<95%; No underline, bold=0%≦CI<50%; and Plain text (no underline, no bold) for no detected peptides. These proteins were outside of Panels A and B, but demonstrated differential expression between AR and NR subjects (pval<0.05) A: PGC 110: Cystatin—C precursor (CST3) IPI00032293.1 (SEQ ID NO: 15). B: PGC138: Sex hormone-binding globulin (SHBG) isoform 2 IPI00219583.1 (SEQ ID NO: 16); SHBG isoform 1 IPI00023019.1 (SEQ ID NO: 17). C: PGC 8: CFH isoform 1 IPI00029739.5 (SEQ ID NO: 18). D: PGC 50: Complement factor I (CFI) precursor IPI00291867.3 (SEQ ID NO: 19); IPI00872555.2 (encoded by cDNA FLJ76262) (SEQ ID NO: 20). E: PGC 48: Serum amyloid P-component precursor IPI00022391.1 (SEQ ID NO: 21).

FIG. 16A-L shows target sequences of 12 nucleic acid markers useful for diagnosis of acute cardiac allograft rejection, listed in Table 6 (SEQ ID NOs: 25-36).

FIG. 17 shows exemplary peptides identified in iTRAQ assays according to some embodiments of the present invention. The list further includes their assigned protein group codes and SEQ ID NOs 37-307.

FIG. 18 A-P shows target sequences of 16 nucleic acid markers useful for diagnosis of acute cardiac allograft rejection in alloreactive T-cells (listed in Table 9) (SEQ ID NOs: 345-360).

FIG. 19 A-Z, AA-KK shows target sequences of 37 nucleic acid markers useful for diagnosis of acute cardiac allograft rejection (listed in Table 10) (SEQ ID NOs: 361-397).

### DETAILED DESCRIPTION

In the description that follows, a number of terms are used extensively, the following definitions are provided to facilitate understanding of various aspects of the invention. Use of examples in the specification, including examples of terms, is for illustrative purposes only and is not intended to limit the scope and meaning of the embodiments of the invention herein. Numeric ranges are inclusive of the numbers defining the range. In the specification, the word “comprising” is used as an open-ended term, substantially equivalent to the phrase “including, but not limited to,” and the word “comprises” has a corresponding meaning.

The present invention provides for methods of diagnosing rejection in a subject that has received a tissue or organ allograft, specifically a cardiac allograft.

The present invention provides genomic, T-cell, nucleic acid, proteomic expression profiles or metabolite profiles related to the assessment, prediction or diagnosis of allograft rejection in a subject. While several of the elements in the genomic or T-cell expression profiles, proteomic expression profiles or metabolite profiles may be individually known in the existing art, the specific combination of the altered expression levels (increased or decreased relative to a control) of specific sets of genomic, T-cell, proteomic or metabolite markers comprise a novel combination useful for assessment, prediction or diagnosis or allograft rejection in a subject.

An allograft is an organ or tissue transplanted between two genetically different subjects of the same species. The subject receiving the allograft is the ‘recipient’, while the subject providing the allograft is the ‘donor’. A tissue or organ allograft may alternately be referred to as a ‘transplant’, a ‘graft’, an ‘allograft’, a ‘donor tissue’ or ‘donor organ’, or similar terms. A transplant between two subjects of different species is a xenograft.

Subjects may present with a variety of symptoms or clinical variables well-known in the literature, however none of these of itself is a predictive or diagnostic of allograft rejection. A myriad of clinical variables may be used in assessing a subject having, or suspected of having, allograft rejection, in addition to biopsy of the allograft. The information gleaned from these clinical variables is then used by a clinician, physician, veterinarian or other practitioner in a clinical field in attempts to determine if rejection is occurring, and how rapidly it progresses, to allow for modification of the immunosuppressive drug therapy of the subject. Examples of clinical variables are described in Table 2.

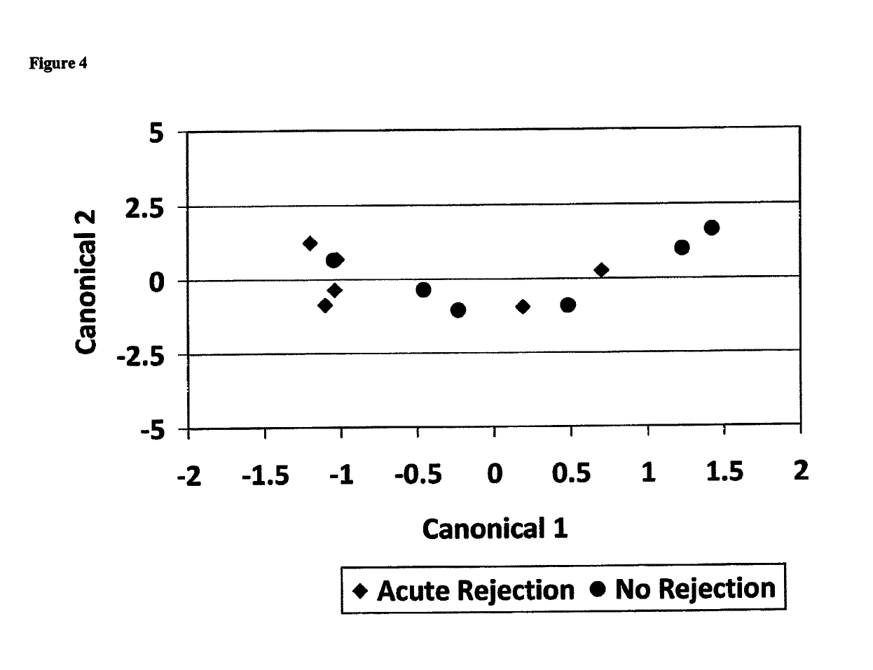

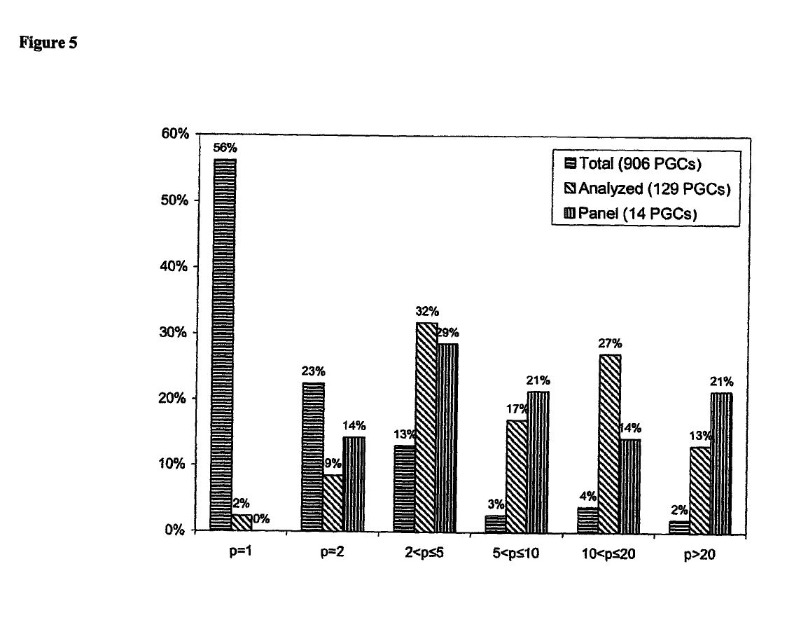

Clinical variables (optionally accompanied by biopsy), while currently the only practical tools available to a clinician in mainstream medical practice, are not always able to cleanly differentiate between an AR (an “acute rejector”) and an NR (a “non-rejector”) subject, as is illustrated in FIG. 4. While the extreme left and right subjects are correctly classified as AR or NR, the bulk of the subjects are represented in the middle range and their status is unclear. This does not negate the value of the clinical variables in the assessment of allograft rejection, but instead indicates their limitation when used in the absence of other methods.

TABLE 2
Clinical variables for possible use in assessment of allograft rejection.

| | Renal/Heart/ | |
|---|---|---|
| Clinical Variable Name | Liver/All | Variable Explanation |
| Primary Diagnosis | All | Diagnosis leading to transplant |
| Secondary Diagnosis | All | Diagnosis leading to transplant |
| “Transplant Procedure - Living | | |
| related, Living unrelated, or | | |
| cadaveric” | | |
| Blood Type | All | Blood Type |
| Blood Rh | All | Blood Rh |
| Height (cm) | All | Height (cm) |
| Weight (kg) | All | Weight (kg) |
| BMI | All | Calculation: Weight/(Height)2 |
| Liver Ascites | All | |
| HLA A1 | All | |
| HLA A2 | All | |
| HLA B1 | All | |
| HLA B2 | All | |
| HLA DR1 | All | |
| HLA DR2 | All | |
| CMV | All | Viral Status |
| CMV Date | All | Date of viral status |
| HIV | All | Viral Status |
| HBV | All | Viral Status |
| HBV Date | All | Date of viral status |
| HbsAb | All | Viral Status |
| HbcAb (Total) | All | Viral Status |
| HBvDNA | All | Viral Status |
| HCV | All | Viral Status |
| HCV Genotype | All | Hepatitis C genotype |
| HCV Genotype Sub | All | “Hepatitis C genotype, subtype” |
| EBV | All | Viral Status |
| Zoster | All | Viral Status |
| Dialysis Start Date | All | Dialysis Start Date |
| Dialysis Type | All | Dialysis Type |
| Cytoxicity Current Level | All | |
| Cytoxicity Current Date | All | |
| Cytoxicity Peak Level | All | |
| Cytoxicity Peak Date | All | |
| Flush Soln | All | Type of Flush Solution used at transplant |
| Cold Time 1 | All | |
| Cold Time 2 | All | |
| Re-Warm Time 1 | All | |
| Re-Warm Time 2 | All | |
| HTLV 1 | All | |
| HTLV 2 | All | |
| HCV RNA | All | |
| 24 hr Urine | All | 24 Hour urine output |
| Systolic Blood Pressure | All | Blood Pressure reading |
| Diastolic Blood Pressure | All | Blood Pressure reading |
| 24 Hr Urine | All | 24 hour urine |
| Sodium | All | Blood test |
| Potassium | All | Blood test |
| Chloride | All | Blood test |
| Total CO2 | All | Blood test |
| Albumin | All | Blood test |
| Protein | All | Blood test |
| Calcium | All | Blood test |
| Inorganic Phosphate | All | Blood test |
| Magnesium | All | Blood test |
| Uric Acid | All | Blood test |
| Glucose | All | Blood test |
| Hemoglobin A1C | All | Blood test |
| CPK | All | Blood test |
| Parathyroid Hormone | All | Blood test |
| Homocysteine | All | Blood test |
| Urine Protein | All | Urine test |
| Creatinine | All | Blood test |
| BUN | All | Blood test |
| Hemaglobin | All | Blood test |
| Platelet Count | All | Blood test |
| WBC Count | All | Blood test |
| Prothrombin Time | All | Blood test |
| Partial Thromboplastin Time | All | Blood test |
| INR | All | Blood test |
| Gamma GT | All | Blood test |
| AST | All | Blood test |
| Alkaline Phosphatase | All | Blood test |
| Amylase | All | Blood test |
| Total Bilirubin | All | Blood test |
| Direct Bilirubin | All | Blood test |
| LDH | All | Blood test |
| ALT | All | Blood test |
| Triglycerides | All | Blood test |
| Cholesterol | All | Blood test |
| HDL Cholesterol | All | Blood test |
| LDL Cholesterol | All | Blood test |
| FEV1 | All | Lung function test |
| FVC | All | Lung function test |
| Total Ferritin | All | Blood test |
| TIBC | All | Blood test |
| Transferrin Saturated | All | Blood test |
| Ferritin | All | Blood test |
| Angiography | Heart | Heart function test |
| Intravascular ultrasound | Heart | Heart function test |
| Dobutamine Stress | Heart | Heart function test |
| Echocardiography | | |
| Cyclosporine WB | All | Immunosuppressive levels |
| Cyclosporine 2 hr | All | Immunosuppressive levels |
| Tacrolimus WB | All | Immunosuppressive levels |
| Sirolimus WB | All | Immunosuppressive total daily dose |
| Solumedrol | All | Immunosuppressive total daily dose |
| Prednisone | All | Immunosuppressive total daily dose |
| Prednisone ALT | All | Immunosuppressive total daily dose |
| Tacrolimus | All | Immunosuppressive total daily dose |
| Cyclosporine | All | Immunosuppressive total daily dose |
| Imuran | All | Immunosuppressive total daily dose |
| Mycophonelate Mofetil | All | Immunosuppressive total daily dose |
| Sirolimus | All | Immunosuppressive total daily dose |
| OKT3 | All | Immunosuppressive total daily dose |
| ATG | All | Immunosuppressive total daily dose |
| ALG | All | Immunosuppressive total daily dose |
| Basiliximab | All | Immunosuppressive total daily dose |
| Daclizumab | All | Immunosuppressive total daily dose |
| Ganciclovir | All | Anti-viral total daily dose |
| Lamivudine | All | Anti-viral total daily dose |
| Riboviron | All | Anti-viral total daily dose |
| Interferon | All | Anti-viral total daily dose |
| Hepatisis C Virus RNA | All | test for presence of HCV values ( ) |
| CMV Antigenemia | All | Antiviral and Virus |
| Valganciclovir | All | Anti-viral total daily dose |
| Neutrophil Number | All | Blood test |
| C Peptide | All | Blood test |
| Peg Interferon | All | Anti-viral total daily dose |
| GFR | All | Glomerular Filtration Rate |
| Complication Events | All | Complication Type |
| Biopsy Scores | Renal | Borderline, 1A, 1B, 2A, 2B, 3, |
| | | Hyperacute |
| Biopsy Scores | Liver | Portal inflammation, Bile duct |
| | | inflammation damage, Venous |
| | | endothelial inflammation each scored |
| | | from 1 to 3 |
| Donor Blood Type | All | Donor Blood Type |
| Donor Blood Rh | All | Donor Rh |
| Donor HLA A1 | All | Donor HLA A1 |
| Donor HLA A2 | All | Donor HLA A2 |
| Donor HLA B1 | All | Donor HLA B1 |
| Donor HLA B2 | All | Donor HLA B2 |
| Donor HLA DR1 | All | Donor HLA DR1 |
| Donor HLA DR2 | All | Donor HLA DR2 |
| Donor CMV | All | Donor CMV |
| Donor HIV | All | Donor HIV |
| Donor HBV | All | Donor HBV |
| Donor HbsAb | All | Donor HbsAb |
| Donor HbcAb (total) | All | Donor HbcAb (total) |
| Donor Hbdna | All | Donor Hbdna |
| Donor HCV | All | Donor HCV |
| Donor EBV | All | Donor EBV |

The multifactorial nature of allograft rejection prediction, diagnosis and assessment is considered in the art to exclude the possibility of a single biomarker that meets even one of the needs of prediction, diagnosis or assessment of allograft rejection. Strategies involving a plurality of markers may take into account this multifactorial nature. Alternately, a plurality of markers may be assessed in combination with clinical variables that are less invasive (e.g. a biopsy not required) to tailor the prediction, diagnosis and/or assessment of allograft rejection in a subject.

Regardless of the methods used for prediction, diagnosis and assessment of allograft rejection, earlier is better—from the viewpoint of preserving organ or tissue function and preventing more systemic detrimental effects. There is no ‘cure’ for allograft rejection, only maintenance of the subject at a suitably immunosuppressed state, or in some cases, replacement of the organ if rejection has progressed too rapidly or is too severe to correct with immunosuppressive drug intervention therapy.

Applying a plurality of mathematical and/or statistical analytical methods to a protein or polypeptide dataset, metabolite concentration data set, or nucleic acid expression dataset may indicate varying subsets of significant markers, leading to uncertainty as to which method is ‘best’ or ‘more accurate’. Regardless of the mathematics, the underlying biology is the same in a dataset. By applying a plurality of mathematical and/or statistical methods to a microarray dataset and assessing the statistically significant subsets of each for common markers, uncertainty may be reduced, and clinically relevant core group of markers may be identified.

“Markers”, “biological markers” or “biomarkers” may be used interchangeably and refer generally to detectable (and in some cases quantifiable) molecules or compounds in a biological sample. A marker may be down-regulated (decreased), up-regulated (increased) or effectively unchanged in a subject following transplantation of an allograft. Markers may include nucleic acids (DNA or RNA), a gene, or a transcript, or a portion or fragment of a transcript in reference to ‘genomic’ markers (alternately referred to as “nucleic acid markers”); polypeptides, peptides, proteins, isoforms, or fragments or portions thereof for ‘proteomic’ markers, or selected molecules, their precursors, intermediates or breakdown products (e.g. fatty acid, amino acid, sugars, hormones, or fragments or subunits thereof) (“metabolite markers” or “metabolomic markers”). In some usages, these terms may reference the level or quantity of a particular protein, peptide, nucleic acid or polynucleotide, or metabolite (in absolute terms or relative to another sample or standard value) or the ratio between the levels of two proteins, polynucleotides, peptides or metabolites, in a subject's biological sample. The level may be expressed as a concentration, for example micrograms per milliliter; as a colorimetric intensity, for example 0.0 being transparent and 1.0 being opaque at a particular wavelength of light, with the experimental sample ranked accordingly and receiving a numerical score based on transmission or absorption of light at a particular wavelength; or as relevant for other means for quantifying a marker, such as are known in the art. In some examples, a ratio may be expressed as a unitless value. A “marker” may also reference to a ratio, or a net value following subtraction of a baseline value. A marker may also be represented as a ‘fold-change’, with or without an indicator of directionality (increase or decrease/up or down). The increase or decrease in expression of a marker may also be referred to as ‘down-regulation’ or ‘up-regulation’, or similar indicators of an increase or decrease in response to a stimulus, physiological event, or condition of the subject. A marker may be present in a first biological sample, and absent in a second biological sample; alternately the marker may be present in both, with a statistically significant difference between the two. Expression of the presence, absence or relative levels of a marker in a biological sample may be dependent on the nature of the assay used to quantify or assess the marker, and the manner of such expression will be familiar to those skilled in the art.

A marker may be described as being differentially expressed when the level of expression in a subject who is rejecting an allograft is significantly different from that of a subject or sample taken from a non-rejecting subject. A differentially expressed marker may be overexpressed or underexpressed as compared to the expression level of a normal or control sample.

A “profile” is a set of one or more markers and their presence, absence, relative level or abundance (relative to one or more controls). For example, a metabolite profile is a dataset of the presence, absence, relative level or abundance of metabolic markers. A proteomic profile is a dataset of the presence, absence, relative level or abundance of proteomic markers. A genomic or nucleic acid profile a dataset of the presence, absence, relative level or abundance of expressed nucleic acids (e.g. transcripts, mRNA, EST or the like). A profile may alternately be referred to as an expression profile.

The increase or decrease, or quantification of the markers in the biological sample may be determined by any of several methods known in the art for measuring the presence and/or relative abundance of a gene product or transcript, or a nucleic acid molecule comprising a particular sequence, polypeptide or protein, metabolite or the like. The level of the markers may be determined as an absolute value, or relative to a baseline value, and the level of the subject's markers compared to a cutoff index (e.g. a non-rejection cutoff index). Alternately the relative abundance of the marker may be determined relative to a control. The control may be a clinically normal subject (e.g. one who has not received an allograft) or may be an allograft recipient that has not previously demonstrated rejection.

In some embodiments, the control may be an autologous control, for example a sample or profile obtained from the subject before undergoing allograft transplantation. In some embodiments, the profile obtained at one time point (before, after or before and after transplantation) may be compared to one or more than one profiles obtained previously from the same subject. By repeatedly sampling the same biological sample from the same subject over time, a composite profile, illustrating marker level or expression over time may be provided. Sequential samples can also be obtained from the subject and a profile obtained for each, to allow the course of increase or decrease in one or more markers to be followed over time For example, an initial sample or samples may be taken before the transplantation, with subsequent samples being taken weekly, biweekly, monthly, bimonthly or at another suitable, regular interval and compared with profiles from samples taken previously. Samples may also be taken before, during and after administration of a course of a drug, for example an immunosuppressive drug.

Techniques, methods, tools, algorithms, reagents and other necessary aspects of assays that may be employed to detect and/or quantify a particular marker or set of markers are varied. Of significance is not so much the particular method used to detect the marker or set of markers, but what markers to detect. As is reflected in the literature, tremendous variation is possible. Once the marker or set of markers to be detected or quantified is identified, any of several techniques may be well suited, with the provision of appropriate reagents. One of skill in the art, when provided with the set of markers to be identified, will be capable of selecting the appropriate assay (for example, a PCR based or a microarray based assay for nucleic acid markers, an ELISA, protein or antibody microarray or similar immunologic assay, or in some examples, use of an iTRAQ, iCAT or SELDI proteomic mass spectrometric based method) for performing the methods disclosed herein.

The present invention provides nucleic acid expression profiles (both genomic and T-cell) proteomic expression profiles and metabolite profiles related to the assessment, prediction or diagnosis of allograft rejection in a subject. While several of the elements in the genomic or T-cell expression profiles, proteomic expression profiles or metabolite profiles may be individually known in the existing art, the specific combination of the altered expression levels (increased or decreased relative to a control) of specific sets of genomic, T-cell, proteomic or metabolite markers comprise a novel combination useful for assessment, prediction or diagnosis of allograft rejection in a subject.

For example, detection or determination, and in some cases quantification, of a nucleic acid may be accomplished by any one of a number methods or assays employing recombinant DNA technologies known in the art, including but not limited to, as sequence-specific hybridization, polymerase chain reaction (PCR), RT-PCR, microarrays and the like. Such assays may include sequence-specific hybridization, primer extension, or invasive cleavage. Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, or the like.

Methods of designing and selecting probes for use in microarrays or biochips, or for selecting or designing primers for use in PCR-based assays are known in the art. Once the marker or markers are identified and the sequence of the nucleic acid determined by, for example, querying a database comprising such sequences, or by having an appropriate sequence provided (for example, a sequence listing as provided herein), one of skill in the art will be able to use such information to select appropriate probes or primers and perform the selected assay.

Standard reference works setting forth the general principles of recombinant DNA technologies known to those of skill in the art include, for example: Ausubel et al, Current Protocols In Molecular Biology, John Wiley & Sons, New York (1998 and Supplements to 2001); Sambrook et al, Molecular Cloning: A Laboratory Manual, 2d Ed., Cold Spring Harbor Laboratory Press, Plainview, N.Y. (1989); Kaufman et al, Eds., Handbook Of Molecular And Cellular Methods In Biology And Medicine, CRC Press, Boca Raton (1995); McPherson, Ed., Directed Mutagenesis: A Practical Approach, IRL Press, Oxford (1991).

Proteins, protein complexes or proteomic markers may be specifically identified and/or quantified by a variety of methods known in the art and may be used alone or in combination. Immunologic- or antibody-based techniques include enzyme-linked immunosorbent assay (ELISA), radioimmunoassay (RIA), western blotting, immunofluorescence, microarrays, some chromatographic techniques (i.e. immunoaffinity chromatography), flow cytometry, immunoprecipitation and the like. Such methods are based on the specificity of an antibody or antibodies for a particular epitope or combination of epitopes associated with the protein or protein complex of interest. Non-immunologic methods include those based on physical characteristics of the protein or protein complex itself. Examples of such methods include electrophoresis, some chromatographic techniques (e.g. high performance liquid chromatography (HPLC), fast protein liquid chromatography (FPLC), affinity chromatography, ion exchange chromatography, size exclusion chromatography and the like), mass spectrometry, sequencing, protease digests, and the like. Such methods are based on the mass, charge, hydrophobicity or hydrophilicity, which is derived from the amino acid complement of the protein or protein complex, and the specific sequence of the amino acids. Examples of methods employing mass spectrometry include those described in, for example, PCT Publication WO 2004/019000, WO 2000/00208, U.S. Pat. No. 6,670,194. Immunologic and non-immunologic methods may be combined to identify or characterize a protein or protein complex. Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, or the like.

Methods of producing antibodies for use in protein or antibody arrays, or other immunology based assays are known in the art. Once the marker or markers are identified and the amino acid sequence of the protein or polypeptide is identified, either by querying of a database or by having an appropriate sequence provided (for example, a sequence listing as provide herein), one of skill in the art will be able to use such information to prepare one or more appropriate antibodies and perform the selected assay.

For preparation of monoclonal antibodies directed towards a biomarker, any technique that provides for the production of antibody molecules by continuous cell lines in culture may be used. Such techniques include, but are not limited to, the hybridoma technique originally developed by Kohler and Milstein (1975, Nature 256:495-497), the trioma technique (Gustafsson et al., 1991, Hum. Antibodies Hybridomas 2:26-32), the human B-cell hybridoma technique (Kozbor et al., 1983, Immunology Today 4:72), and the EBV hybridoma technique to produce human monoclonal antibodies (Cole et al., 1985, In: Monoclonal Antibodies and Cancer Therapy, Alan R. Liss, Inc., pp. 77-96). Human antibodies may be used and can be obtained by using human hybridomas (Cote et al., 1983, Proc. Natl. Acad. Sci. USA 80:2026-2030) or by transforming human B cells with EBV virus in vitro (Cole et al., 1985, In: Monoclonal Antibodies and Cancer Therapy, Alan R. Liss, Inc., pp. 77-96). Techniques developed for the production of “chimeric antibodies” (Morrison et al, 1984, Proc. Natl. Acad. Sci. USA 81:6851-6855; Neuberger et al, 1984, Nature 312:604-608; Takeda et al, 1985, Nature 314:452-454) by splicing the genes from a mouse antibody molecule specific for a biomarker together with genes from a human antibody molecule of appropriate biological activity can be used; such antibodies are within the scope of this invention. Techniques described for the production of single chain antibodies (U.S. Pat. No. 4,946,778) can be adapted to produce a biomarker-specific antibodies. An additional embodiment of the invention utilizes the techniques described for) the construction of Fab expression libraries (Huse et al, 1989, Science 246:1275-1281) to allow rapid and easy identification of monoclonal Fab fragments with the desired specificity for a biomarker proteins. Non-human antibodies can be “humanized” by known methods (e.g., U.S. Pat. No. 5,225,539).

Antibody fragments that contain the idiotypes of a biomarker can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments. Synthetic antibodies, e.g., antibodies produced by chemical synthesis, are useful in the present invention

Standard reference works described herein and known to those skilled in the relevant art describe both immunologic and non-immunologic techniques, their suitability for particular sample types, antibodies, proteins or analyses. Standard reference works setting forth the general principles of immunology and assays employing immunologic methods known to those of skill in the art include, for example: Harlow and Lane, Antibodies: A Laboratory Manual, 2d Ed., Cold Spring Harbor Laboratory Press, Cold Spring Harbor, N.Y. (1999); Harlow and Lane, Using Antibodies: A Laboratory Manual. Cold Spring Harbor Laboratory Press, New York; Coligan et al. eds. Current Protocols in Immunology, John Wiley & Sons, New York, N.Y. (1992-2006); and Roitt et al., Immunology, 3d Ed., Mosby-Year Book Europe Limited, London (1993).

Standard reference works setting forth the general principles of peptide synthesis technology and methods known to those of skill in the art include, for example: Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; Sambrook et al., Molecular Cloning: A Laboratory Manual, 3 ed., Cold Spring Harbor Press, Cold Spring Harbor, N.Y. 2001; and Ausubel et al., Current Protocols in Molecular Biology, Greene Publishing Associates and John Wiley & Sons, NY, 1994).

A subject's rejection status may be described as an “acute rejector” (AR) or as a “non-rejector” (NR) and is determined by comparison of the concentration of the markers to that of a non-rejector cutoff index. A “non-rejector cutoff index” is a numerical value or score, beyond or outside of which a subject is categorized as having an AR rejection status. The non-rejector cutoff index may be alternately referred to as a ‘control value’, a ‘control index’, or simply as a ‘control’. A non-rejector cutoff-index may be the concentration of individual markers in a control subject population and considered separately for each marker measured; alternately the non-rejector cutoff index may be a combination of the concentration of the markers, and compared to a combination of the concentration of the markers in the subject's sample provided for diagnosing. The control subject population may be a normal or healthy control population, or may be an allograft recipient population that has not, or is not, rejecting the allograft. The control may be a single subject, and for some embodiments, may be an autologous control. A control, or pool of controls, may be constant e.g. represented by a static value, or may be cumulative, in that the sample population used to obtain it may change from site to site, or over time and incorporate additional data points. For example, a central data repository, such as a centralized healthcare information system, may receive and store data obtained at various sites (hospitals, clinical laboratories or the like) and provide this cumulative data set for use with the methods of the invention at a single hospital, community clinic, for access by an end user (i.e. an individual medical practitioner, medical clinic or center, or the like).

The non-rejector cutoff index may be alternately referred to as a ‘control value’, a ‘control index’ or simply as a ‘control’. In some embodiments the cutoff index may be further characterized as being a metabolite cutoff index (for metabolite profiling of subjects), a genomic cutoff index (for genomic expression profiling of subjects), a proteomic cutoff index (for proteomic profiling of subjects), or the like.

A “biological sample” refers generally to body fluid or tissue or organ sample from a subject. For example, the biological sample may a body fluid such as blood, plasma, lymph fluid, serum, urine or saliva. A tissue or organ sample, such as a non-liquid tissue sample may be digested, extracted or otherwise rendered to a liquid form—examples of such tissues or organs include cultured cells, blood cells, skin, liver, heart, kidney, pancreas, islets of Langerhans, bone marrow, blood, blood vessels, heart valve, lung, intestine, bowel, spleen, bladder, penis, face, hand, bone, muscle, fat, cornea or the like. A plurality of biological samples may be collected at any one time. A biological sample or samples may be taken from a subject at any time, including before allograft transplantation, at the time of translation or at anytime following transplantation. A biological sample may comprise nucleic acid, such as deoxyribonucleic acid or ribonucleic acid, or a combination thereof, in either single or double-stranded form. When an organ is removed from a donor, the spleen of the donor or a part of it may be kept as a biological sample from which to obtain donor T-cells. When an organ is removed from a living donor, a blood sample may be taken, from which donor T-cells may be obtained. Alloreactive T-cells may be isolated by exploiting their specific interaction with antigens (including the MHC complexes) of the allograft. Methods to enable specific isolation of alloreactive T-cells are described in, for example PCT Publication WO 2005/05721, herein incorporated by reference.

A lymphocyte is nucleated or ‘white’ blood cell (leukocyte) of lymphoid stem cell origin. Lymphocytes include T-cells, B-cells natural killer cells and the like, and other immune regulatory cells. A “T-cell” is a class of lymphocyte responsible for cell-mediated immunity, and for stimulating B-cells. A stimulated B-cell produces antibodies for specific antigens. Both B-cells and T-cells function to recognize non-self antigens in a subject. Non-self antigens include those of viruses, bacteria and other infectious agents as well as allografts.

An alloreactive T-cell is a T-cell that is activated in response to an alloantigen. A T-cell that is reactive to a xenoantigen is a xenoreactive T-cell. A xenoantigen is an antigen from another species or species' tissue, such as a xenograft. Alloreactive T cells are the front-line of the graft rejection immune response. They are a subset (−0.1-1%) of the peripheral blood mononuclear cells (PBMC) which recognize allogeneic antigens present on the foreign graft. They may infiltrate the foreign graft, to initiate a cascade of anti-graft immune response, which, if unchecked, will lead to rejection and failure of the graft. Alloreactive T cells, therefore provide specificity compared to other sources of markers, or may function as a complementary source of markers that differentiate between stages of organ rejection.

The term “subject” or “patient” generally refers to mammals and other animals including humans and other primates, companion animals, zoo, and farm animals, including, but not limited to, cats, dogs, rodents, rats, mice, hamsters, rabbits, horses, cows, sheep, pigs, goats, poultry, etc. A subject includes one who is to be tested, or has been tested for prediction, assessment or diagnosis of allograft rejection. The subject may have been previously assessed or diagnosed using other methods, such as those described herein or those in current clinical practice, or may be selected as part of a general population (a control subject).

A fold-change of a marker in a subject, relative to a control may be at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 or more, or any amount therebetween. The fold change may represent a decrease, or an increase, compared to the control value.

One or more than one includes 1, 2, 3, 4, 5, 6, 7, 8, 9, 10, 11, 12, 13, 14, 15, 16 or more.

“Down-regulation” or ‘down-regulated may be used interchangeably and refer to a decrease in the level of a marker, such as a gene, nucleic acid, metabolite, transcript, protein or polypeptide. “Up-regulation” or “up-regulated” may be used interchangeably and refer to an increase in the level of a marker, such as a gene, nucleic acid, metabolite, transcript, protein or polypeptide. Also, a pathway, such as a signal transduction or metabolic pathway may be up- or down-regulated.

Once a subject is identified as an acute rejector, or at risk for becoming an acute rejector by any method (genomic, proteomic, metabolomic or a combination thereof), therapeutic measures may be implemented to alter the subject's immune response to the allograft. The subject may undergo additional monitoring of clinical values more frequently, or using more sensitive monitoring methods. Additionally the subject may be administered immunosuppressive medicaments to decrease or increase the subject's immune response. Even though a subject's immune response needs to be suppressed to prevent rejection of the allograft, a suitable level of immune function is also needed to protect against opportunistic infection. Various medicaments that may be administered to a subject are known; see for example, Goodman and Gilman's The Pharmacological Basis of Therapeutics 11^{th} edition. Ch 52, pp 1405-1431 and references therein; L L Brunton, J S Lazo, K L Parker editors. Standard reference works setting forth the general principles of medical physiology and pharmacology known to those of skill in the art include: Fauci et al., Eds., Harrison's Principles Of Internal Medicine, 14th Ed., McGraw-Hill Companies, Inc. (1998). Other preventative and therapeutic strategies are reviewed in the medical literature—see, for example Kobashigawa et al. 2006. Nature Clinical Practice. Cardiovascular Medicine 3:203-21.

Genomic Nucleic Acid Expression Profiling

A method of diagnosing acute allograft rejection in a subject as provided by the present invention comprises 1) determining the expression profile of one or more than one nucleic acid markers in a biological sample from the subject, the nucleic acid markers selected from the group comprising TRF2, SRGAP2P1, KLF4, YLPM1, BID, MARCKS, CLEC2B, ARHGEF7, LYPLAL1, WRB, FGFR1OP2, MBD4; 2) comparing the expression profile of the one or more than one nucleic acid markers to a non-rejector profile; and 3) determining whether the expression level of the one or more than one nucleic acid markers is up-regulated or down-regulated relative to the control profile, wherein up-regulation or down-regulation of the one or more than one nucleic acid markers is indicative of the rejection status.

Therefore, the invention also provides for a method of predicting, assessing or diagnosing allograft rejection in a subject as provided by the present invention comprises 1) measuring the increase or decrease of one or more than one nucleic acid markers selected from the group comprising TRF2, SRGAP2P1, KLF4, YLPM1, BID, MARCKS, CLEC2B, ARHGEF7, LYPLAL1, WRB, FGFR1OP2, MBD4; and 2) determining the ‘rejection status’ of the subject, wherein the determination of ‘rejection status’ of the subject is based on comparison of the subject's nucleic acid marker expression profile to a control nucleic acid marker expression profile.

The phrase “gene expression data”, “gene expression profile” “nucleic acid expression profile” or “marker expression profile” as used herein refers to information regarding the relative or absolute level of expression of a gene or set of genes in a biological sample. The level of expression of a gene may be determined based on the level of a nucleic acid such as RNA including mRNA, transcribed from or encoded by the gene.

A “polynucleotide”, “oligonucleotide”, “nucleic acid” or “nucleotide polymer” as used herein may include synthetic or mixed polymers of nucleic acids, including RNA, DNA or both RNA and DNA, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), and modified linkages (e.g., alpha anomeric polynucleotides, etc.). Also included are synthetic molecules that mimic polynucleotides in their ability to bind to a designated sequence via hydrogen bonding and other chemical interactions.

An oligonucleotide includes variable length nucleic acids, which may be useful as probes, primers and in the manufacture of microarrays (arrays) for the detection and/or amplification of specific nucleic acids. Oligonucleotides may comprise DNA, RNA, PNA or other polynucleotide moieties as described in, for example, U.S. Pat. No. 5,948,902. Such DNA, RNA or oligonucleotide strands may be synthesized by the sequential addition (5′-3′ or 3′-5′) of activated monomers to a growing chain which may be linked to an insoluble support. Numerous methods are known in the art for synthesizing oligonucleotides for subsequent individual use or as a part of the insoluble support, for example in arrays (BERNFIELD M R. and ROTTMAN F M. J. Biol. Chem. (1967) 242(18):4134-43; SULSTON J. et al. PNAS (1968) 60(2):409-415; GILLAM S. et al. Nucleic Acid Res. (1975) 2(5):613-624; BONORA G M. et al. Nucleic Acid Res. (1990) 18(11):3155-9; LASHKARI D A. et al. PNAS (1995) 92(17):7912-5; MCGALL G. et al. PNAS (1996) 93(24):13555-60; ALBERT T J. et al. Nucleic Acid Res. (2003) 31(7):e35; GAO X. et al. Biopolymers (2004) 73(5):579-96; and MOORCROFT M J. et al. Nucleic Acid Res. (2005) 33(8):e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

A “gene” is an ordered sequence of nucleotides located in a particular position on a particular chromosome that encodes a specific functional product and may include untranslated and untranscribed sequences in proximity to the coding regions (5′ and 3′ to the coding sequence). Such non-coding sequences may contain regulatory sequences needed for transcription and translation of the sequence or splicing of introns, for example, or may as yet to have any function attributed to them beyond the occurrence of the mutation of interest. A gene may also include one or more promoters, enhancers, transcription factor binding sites, termination signals or other regulatory elements. A gene may be generally referred to as ‘nucleic acid’.

The term “microarray,” “array,” or “chip” refers to a plurality of defined nucleic acid probes coupled to the surface of a substrate in defined locations. The substrate may be preferably solid. Microarrays, their methods of manufacture, use and analysis have been generally described in the art in, for example, U.S. Pat. Nos. 5,143,854 (Pirrung), 5,424,186 (Fodor), 5,445,934 (Fodor), 5,677,195 (Winkler), 5,744,305 (Fodor), 5,800,992 (Fodor), 6,040,193 (Winkler), and Fodor et al. 1991. Science, 251:767-777.

‘Hybridization” includes a reaction in which one or more polynucleotides and/or oligonucleotides interact in an ordered manner (sequence-specific) to form a complex that is stabilized by hydrogen bonding—also referred to as ‘Watson-Crick’ base pairing. Variant base-pairing may also occur through non-canonical hydrogen bonding includes Hoogsteen base pairing. Under some thermodynamic, ionic or pH conditions, triple helices may occur, particularly with ribonucleic acids. These and other variant hydrogen bonding or base-pairing are known in the art, and may be found in, for example, Lehninger—Principles of Biochemistry, 3^{rd} edition (Nelson and Cox, eds. Worth Publishers, New York.).

Hybridization reactions can be performed under conditions of different “stringency”. The stringency of a hybridization reaction includes the difficulty with which any two nucleic acid molecules will hybridize to one another. Stringency may be increased, for example, by increasing the temperature at which hybridization occurs, by decreasing the ionic concentration at which hybridization occurs, or a combination thereof. Under stringent conditions, nucleic acid molecules at least 60%, 65%, 70%, 75% or more identical to each other remain hybridized to each other, whereas molecules with low percent identity cannot remain hybridized. An example of stringent hybridization conditions are hybridization in 6× sodium chloride/sodium citrate (SSC) at about 44-45° C., followed by one or more washes in 0.2×SSC, 0.1% SDS at 50° C., 55° C., 60° C., 65° C., or at a temperature therebetween.

Hybridization between two nucleic acids may occur in an antiparallel configuration—this is referred to as ‘annealing’, and the paired nucleic acids are described as complementary. A double-stranded polynucleotide may be “complementary”, if hybridization can occur between one of the strands of the first polynucleotide and the second. The degree of which one polynucleotide is complementary with another is referred to as homology, and is quantifiable in terms of the proportion of bases in opposing strands that are expected to hydrogen bond with each other, according to generally accepted base-pairing rules.

In general, sequence-specific hybridization involves a hybridization probe, which is capable of specifically hybridizing to a defined sequence. Such probes may be designed to differentiate between sequences varying in only one or a few nucleotides, thus providing a high degree of specificity. A strategy which couples detection and sequence discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Probes used in hybridization may include double-stranded DNA, single-stranded DNA and RNA oligonucleotides, and peptide nucleic acids. Hybridization conditions and methods for identifying markers that hybridize to a specific probe are described in the art—see, for example, Brown, T. “Hybridization Analysis of DNA Blots” in Current Protocols in Molecular Biology. F M Ausubel et al, editors. Wiley & Sons, 2003. doi: 10.1002/0471142727.mb0210s21. Suitable hybridization probes for use in accordance with the invention include oligonucleotides, polynucleotides or modified nucleic acids from about 10 to about 400 nucleotides, alternatively from about 20 to about 200 nucleotides, or from about 30 to about 100 nucleotides in length.

Specific sequences may be identified by hybridization with a primer or a probe, and this hybridization subsequently detected.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are well known in the art, and are taught, for example, in Beverly, S M. Enzymatic Amplification of RNA by PCR (RT-PCR) in Current Protocols in Molecular Biology. F M Ausubel et al, editors. Wiley & Sons, 2003. doi: 10.1002/0471142727.mb1505s56. Synthesis of the replicate copies may include incorporation of a nucleotide having a label or tag, for example, a fluorescent molecule, biotin, or a radioactive molecule. The replicate copies may subsequently be detected via these tags, using conventional methods.

A primer may also be used as a probe in hybridization reactions, such as Southern or Northern blot analyses (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

A “probe set” (or ‘primer set’) as used herein refers to a group of oligonucleotides that may be used to detect one or more expressed nucleic acids, or expressed genes. Detection may be, for example, through amplification as in PCR and RT-PCR, or through hybridization, as on a microarray, or through selective destruction and protection, as in assays based on the selective enzymatic degradation of single or double stranded nucleic acids. Probes in a probe set may be labeled with one or more fluorescent, radioactive or other detectable moieties (including enzymes). Probes may be any size so long as the probe is sufficiently large to selectively detect the desired gene—generally a size range from about 15 to about 25, or to about 30 nucleotides is of sufficient size. A probe set may be in solution, e.g. for use in multiplex PCR. Alternately, a probe set may be adhered to a solid surface, as in an array or microarray.

In some embodiments of the invention, a probe set for detection of nucleic acids expressed by a set of genomic markers comprising one or more of TRF2, SRGAP2P1, KLF4, YLPM1, BID, MARCKS, CLEC2B, ARHGEF7, LYPLAL1, WRB, FGFR1OP2, and MBD4 is provided. Such a probe set may be useful for determining the rejection status of a subject. The probe set may comprise one or more pairs of primers for specific amplification (e.g. PCR or RT-PCR) of nucleic acid sequences corresponding to one or more of TRF2, SRGAP2P1, KLF4, YLPM1, BID, MARCKS, CLEC2B, ARHGEF7, LYPLAL1, WRB, FGFR1OP2 and MBD4. In another embodiment of the invention, the probe set is part of a microarray.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput array (BORTOLIN S. et al. 2004 Clinical Chemistry 50: 2028-36). This method amplifies genomic DNA by PCR followed by sequence-specific primer extension with universally tagged primers. The products are then sorted on a Tag-It array and detected using the Luminex xMAP system.

It will be appreciated by a person of skill in the art that any numerical designations of nucleotides or amino acids within a sequence are relative to the specific sequence. Also, the same positions may be assigned different numerical designations depending on the way in which the sequence is numbered and the sequence chosen. Furthermore, sequence variations such as insertions or deletions, may change the relative position and subsequently the numerical designations of particular nucleotides or amino acids at or around a mutational site. For example, the sequences represented by accession numbers AC006825.13, AC016026.15, AY309933.2, AY4771193.1, CQ786436.1, AF042083.1, AF087891.1, AK094795.1, AY005151.1, BC009197.2, BM842561.1, BQ068464.1, CR407603.1, CR600736.1, NM_{—}00196.2 all represent human BID nucleotide sequences, but may have some sequence differences, and numbering differences between them. As another example, the sequences represented by accession numbers NP_{—}932070.1, NP_{—}932071.1, NP_{—}001187.1, EAW57770.1, CAG17894.1, AAC34365.1, AAP97190.1, AAQ15216.1, AAH36364.1, CAG28531.1, P55957.1 all represent human BID polypeptide sequences, but may have some sequence differences, and numbering differences between them.

Selection and/or design of probes, primers or probe sets for specific detection of expression of any gene of interest, including any of the above genes is within the ability of one of skill in the relevant art, when provided with one or more nucleic acid sequences of the gene of interest. Further, any of several probes, primers or probe sets, or a plurality of probes, primers or probe sets may be used to detect a gene of interest, for example, an array may include multiple probes for a single gene transcript—the aspects of the invention as described herein are not limited to any specific probes exemplified.

Sequence identity or sequence similarity may be determined using a nucleotide sequence comparison program (for DNA or RNA sequences, or fragments or portions thereof) or an amino acid sequence comparison program (for protein, polypeptide or peptide sequences, or fragments or portions thereof), such as that provided within DNASIS (for example, but not limited to, using the following parameters: GAP penalty 5, # of top diagonals 5, fixed GAP penalty 10, k-tuple 2, floating gap 10, and window size 5). However, other methods of alignment of sequences for comparison are well-known in the art for example the algorithms of Smith & Waterman (1981, Adv. Appl. Math. 2:482), Needleman & Wunsch (J. Mol. Biol. 48:443, 1970), Pearson & Lipman (1988, Proc. Nat'l. Acad. Sci. USA 85:2444), and by computerized implementations of these algorithms (e.g. GAP, BESTFIT, FASTA, and BLAST), or by manual alignment and visual inspection.

If a nucleic acid or gene, polypeptide or sequence of interest is identified and a portion or fragment of the sequence (or sequence of the gene polypeptide or the like) is provided, other sequences that are similar, or substantially similar may be identified using the programs exemplified above. For example, when constructing a microarray or probe sequences, the sequence and location are known, such that if a microarray experiment identifies a ‘hit’ (the probe at a particular location hybridizes with one or more nucleic acids in a sample, the sequence of the probe will be known (either by the manufacturer or producer of the microarray, or from a database provided by the manufacturer—for example the NetAffx databases of Affymetrix, the manufacturer of the Human Genome U133 Plus 2.0 Array). If the identity of the sequence source is not provided, it may be determined by using the sequence of the probe in a sequence-based search of one or more databases. For peptide or peptide fragments identified by proteomics assays, for example iTRAQ, the sequence of the peptide or fragment may be used to query databases of amino acid sequences as described above. Examples of such a database include those maintained by the National Centre for Biotechnology Information, or those maintained by the European Bioinformatics Institute.

A protein or polypeptide, nucleic acid or fragment or portion thereof may be considered to be specifically identified when its sequence may be differentiated from others found in the same phylogenetic Species, Genus, Family or Order. Such differentiation may be identified by comparison of sequences. Comparisons of a sequence or sequences may be done using a BLAST algorithm (Altschul et al. 1009. J. Mol Biol 215:403-410). A BLAST search allows for comparison of a query sequence with a specific sequence or group of sequences, or with a larger library or database (e.g. GenBank or GenPept) of sequences, and identify not only sequences that exhibit 100% identity, but also those with lesser degrees of identity. For example, regarding a protein with multiple isoforms (either resulting from, for example, separate genes or variant splicing of the nucleic acid transcript from the gene, or post translational processing), an isoform may be specifically identified when it is differentiated from other isoforms from the same or a different species, by specific detection of a structure, sequence or motif that is present on one isoform and is absent, or not detectable on one or more other isoforms.

Access to the methods of the invention may be provided to an end user by, for example, a clinical laboratory or other testing facility performing the individual marker tests—the biological samples are provided to the facility where the individual tests and analyses are performed and the predictive method applied; alternately, a medical practitioner may receive the marker values from a clinical laboratory and use a local implementation or an internet-based implementation to access the predictive methods of the invention.

Determination of statistical parameters such as multiples of the median, standard error, standard deviation and the like, as well as other statistical analyses as described herein are known and within the skill of one versed in the relevant art. Use of a particular coefficient, value or index is exemplary only and is not intended to constrain the limits of the various aspects of the invention as disclosed herein.

Interpretation of the large body of gene expression data obtained from, for example, microarray experiments, or complex RT-PCR experiments may be a formidable task, but is greatly facilitated through use of algorithms and statistical tools designed to organize the data in a way that highlights systematic features. Visualization tools are also of value to represent differential expression by, for example, varying intensity and hue of colour (Eisen et al. 1998. Proc Natl Acad Sci 95:14863-14868). The algorithm and statistical tools available have increased in sophistication with the increase in complexity of arrays and the resulting datasets, and with the increase in processing speed, computer memory, and the relative decrease in cost of these.

Mathematical and statistical analysis of nucleic acid or protein expression profiles, or metabolite profiles may accomplish several things—identification of groups of genes that demonstrate coordinate regulation in a pathway or a domain of a biological system, identification of similarities and differences between two or more biological samples, identification of features of a gene expression profile that differentiate between specific events or processes in a subject, or the like. This may include assessing the efficacy of a therapeutic regimen or a change in a therapeutic regimen, monitoring or detecting the development of a particular pathology, differentiating between two otherwise clinically similar (or almost identical) pathologies, or the like.

Clustering methods are known and have been applied to microarray datasets, for example, hierarchical clustering, self-organizing maps, k-means or deterministic annealing. (Eisen et al, 1998 Proc Natl Mad Sci USA 95:14863-14868; Tamayo, P., et al. 1999. Proc Natl Acad Sci USA 96:2907-2912; Tavazoie, S., et al. 1999. Nat Genet. 22:281-285; Alon, U., et al. 1999. Proc Natl Acad Sci USA 96:6745-6750). Such methods may be useful to identify groups of genes in a gene expression profile that demonstrate coordinate regulation, and also useful for the identification of novel genes of otherwise unknown function that are likely to participate in the same pathway or system as the others demonstrating coordinate regulation.

The pattern of nucleic acid or protein expression in a biological sample may also provide a distinctive and accessible molecular picture of its functional state and identity (DeRisi 1997; Cho 1998; Chu 1998; Holstege 1998; Spellman 1998). Two different samples that have related gene expression patterns are therefore likely to be biologically and functionally similar to one another, conversely two samples that demonstrate significant differences may not only be differentiated by the complex expression pattern displayed, but may indicate a diagnostic subset of gene products or transcripts that are indicative of a specific pathological state or other physiological condition, such as allograft rejection.

Applying a plurality of mathematical and/or statistical analytical methods to a microarray dataset may indicate varying subsets of significant markers, leading to uncertainty as to which method is ‘best’ or ‘more accurate’. Regardless of the mathematics, the underlying biology is the same in a dataset. By applying a plurality of mathematical and/or statistical methods to a microarray dataset and assessing the statistically significant subsets of each for common markers to all, the uncertainty is reduced, and clinically relevant core group of markers is identified.

Genomic Expression Profiling Markers (“Genomic Markers”)

The present invention provides for a core group of markers useful for the assessment, prediction or diagnosis of allograft rejection, including acute allograft rejection, comprising TRF2, SRGAP2P1, KLF4, YLPM1, BID, MARCKS, CLEC2B, ARHGEF7, LYPLAL1, WRB, FGFR1OP2, MBD4.

Of the 39 genes or transcripts (Table 6) that were detected, quantified and found to demonstrate a statistically significant fold change in the AR samples relative to non-rejecting transplant (NR) controls for at least one of the three modified t-tests applied, 12 markers are in the union set (statistically significant for all three tests). The fold change for each marker in the larger set of 39 was at least two-fold, and may represent an increase/up-regulation or decrease/down-regulation of the gene or transcript in question.

The product of the Transferrin receptor 2 (TFR2) gene mediates cellular uptake of transferrin-bound iron in a non-iron dependent manner. TFR2 may be involved in iron metabolism, hepatocyte function and erythrocyte development and differentiation. Nucleotide sequences of human TFR2 are known (e.g. GenBank Accession No. AF053356, AK022002, AK000421).

SLIT-ROBO Rho GTPase activating protein 2 Pseudogene 1 (SRGAP2P1) is a pseudogene demonstrating sequence similarity to SRGAP2. Nucleotide sequences of human SRGAP2P1 are known (e.g. GenBank Accession No. AL358175.18, BC017972.1, BC036880.1, BC112927.1, DQ786311.1).

The product of the Kruppel-like factor 4 (KLF4) gene may function as an activator or repressor of transcription. Nucleotide sequences of human KLF4 are known (e.g. GenBank Accession No. CH410015.1, DQ658241.1, AF022184.1, AK095134.1).

The product of the YLP motif containing 1 (YLPM1) gene may have a role in modulation of telomerate activity and cell division. Nucleotide sequences of human YLPM1 are known (e.g. GenBank Accession No. AK095760.1, AC006530.4, AC007956.5, AL832365.1, BC007792.1).

The BH3 interacting domain death agonist (BID) gene encodes a death agonist that heterodimerizes with either agonist BAX or antagonist BCL2. The encoded protein is a member of the BCL-2 family of cell death regulators. It is a mediator of mitochondrial damage induced by caspase-8. Nucleotide sequences of human BID are known (e.g. GenBank Accession No. AC006825.13, AF042083.1, AF087891.1, AK094795.1).

The product of the myristoylated alanine-rich protein kinase C substrate (MARCKS) gene is an actin filament crosslinking protein and a substrate for protein kinase C. Phosphorylation by protein kinase C or binding to calcium-calmodulin inhibits its association with actin and with the plasma membrane, leading to its presence in the cytoplasm. The protein is thought to be involved in cell motility, phagocytosis, membrane trafficking and mitogenesis. Nucleotide sequences of human MARCKS are known (e.g. GenBank Accession No. AL132660.14, CH471051.2, AI142997.1, BC013004.2).

The C-type lectin domain family 2, member B (CLEC2B) gene encodes a member of the C-type lectin/C-type lectin-like domain (CTL/CTLD) superfamily. Members of this family share a common protein fold and have diverse functions, such as cell adhesion, cell-cell signalling, glycoprotein turnover, and roles in inflammation and immune response. The encoded type 2 transmembrane protein may function as a cell activation antigen. Nucleotide sequences of human CLEC2B are known (e.g. GenBank Accession No. CH471094.1, AC007068.17, AY142147.1, BC005254.1).

The Rho guanine nucleotide exchange factor (GEF, ARHGEF7, BETA-PIX) gene encodes a member of the Rho guanine nucleotide exchange factor family. Nucleotide sequences of human BETA-PIX are known (e.g. GenBank Accession No. BC050521.1, NM_{—}003899.3).

Lysophospholipase-like 1 (LYPLAL1)—nucleotide sequences of human LYPLAL1 are known (e.g. GenBank Accession No. CH471100.2, AK291542.1, AY341430.1, BC016711.1)

The Tryptophan rich basic protein (WRB) gene encodes a basic nuclear protein of unknown function, widely expressed in adult and fetal tissues. Nucleotide sequences of human WRB are known (e.g. GenBank Accession No. AL163279.2, CH471079.2, AK293113.1, BC012415.1).

FGFR1 oncogene partner 2 (FGFR1OP2) is a fusion gene involving a chromosome 12×8 translocation, identified in an 8; 11 myeloproliferative syndrome patient. Nucleotide sequences of human FGR1OP2 are known (e.g. GenBank Accession No. CH471094.1, AF161472.1, AK001534.1, AL117608.1).

The product of the methyl-CpG binding domain protein 4 (MBD4) gene encodes a nuclear protein having a methyl-CpG binding domain, and capable of binding specifically to methylated DNA. Sequence similarities suggest a role in DNA repair. Nucleotide sequences of human MBD4 are known (e.g. GenBank Accession No. AF120999.1, CH471052.2, AF072250.1, AF532602.1)

Biological Pathways Associated with Genomic Biomarkers of the Invention

Biomarkers of the present invention are associated with biological pathways that may be particularly affected by the immune processes and a subject's response to an allograft rejection. FIG. 3 illustrates a pathway-based relationship between the biomarkers ARHGEF7, TRF2, BID, MARCKS, KLF4, CLEC2B and MBD4. Examples of pathways include:

1. BETAPIX→4 Rac1→4 STAT1→KLF4

2. KLF4→(c-MYC→4 CREB1)→CLECSF2

3. STAT1→BID

4. KLF→Beta-catenin→HDAC1→MBD4

5. BETA-PIX→CDC42→PKC-zeta4→MARCKS

6. KLF4→SP1→HLA-H→TfR2

ARHGEF7, TRF2, BID, MARCKS, KLF4, CLEC2B and MBD4 may, therefore, have a biological role in the allograft rejection process, and represent a therapeutic target.

Large scale gene expression analysis methods, such as microarrays have indicated that groups of genes that have an interaction (often with two or more degrees of separation) are expressed together and may have common regulatory elements. Other examples of such coordinate regulation are known in the art, see, for example, the diauxic shift of yeast (DiRisi et al 1997 Science 278:680-686; Eisen et al. 1998. Proc Natl Acad Sci 95:14863-14868).

BID is one of the gene products whose transcript demonstrates a statistically significant difference between an AR and NR subject. It is known that BID is cleaved into active fragments during ischemia/reperfusion in an animal model (Chen et al 2001. J. Biol Chem 276:30724-8). The decrease in BID transcripts observed in AR subjects compared to NR subjects suggests that BID may have a key effect in the cellular events occurring during organ rejection, but the pathways through which BID exerts its effect may be unexpected. Other markers exhibiting differential expression between AR and NR subjects that may interact with BID, or interact with an interactor of BID and thus participate in the pathway or pathways triggered by allograft rejection include, but are not limited to, FasR (CD95), FLASH, Caspase-8, HGK (MAP4K4), MEKK1 (MAP3K1) and Myosin Va. BID may, therefore, have a biological role in the allograft rejection process, and represent a therapeutic target.

BETA-PIX is another of the gene products whose transcript demonstrates a statistically significant difference between an AR and NR subject. It is known that a variety of signaling molecules are affected by, or affect, the cyclic AMP-dependent protein kinase (PKA) pathway to regulate cellular behaviors, including intermediary metabolism, ion channel conductivity, and transcription. PKA plays a central role in cytoskeletal regulation and cell migration. Other markers that may interact with BETA-PIX, or interact with an interactor of BETA-PIX and thus participate in the pathway or pathways triggered by allograft rejection include, but are not limited to, ITGA4 (Integrin alpha 4), ITGB1 (Integrin beta 1), ADCY7 (Adenylate cyclase), PRKACB (PKA catalytic subunit), PRKAR1A (PKA regulatory subunit), RAC1, RhoA, PPP1R12A (MLCP (regulatory subunit)), MYL4 (MELC). BETA-PIX may, therefore, have a biological role in the allograft rejection process, and represent a therapeutic target.

Without wishing to be bound by theory, other genes or transcript described herein, for example TRF2, SRGAP2P1, KLF4, YLPM1, BID, MARCKS, CLEC2B, ARHGEF7, LYPLAL1, WRB, FGFR1OP2 or MBD4 may have a biological role in the allograft rejection process, and represent a therapeutic target

The invention also provides for a kit for use in predicting or diagnosing a subject's rejection status. The kit may comprise reagents for specific and quantitative detection of TRF2, SRGAP2P1, KLF4, YLPM1, BID, MARCKS, CLEC2B, ARHGEF7, LYPLAL1, WRB, FGFR1OP2, MBD4, along with instructions for the use of such reagents and methods for analyzing the resulting data. The kit may be used alone for predicting or diagnosing a subject's rejection status, or it may be used in conjunction with other methods for determining clinical variables, or other assays that may be deemed appropriate. The kit may include, for example, one or more labelled oligonucleotides capable of selectively hybridizing to the marker. The kit may further include, for example, one or more oligonucleotides operable to amplify a region of the marker (e.g. by PCR). Instructions or other information useful to combine the kit results with those of other assays to provide a non-rejection cutoff index for the prediction or diagnosis of a subject's rejection status may also be provided.

### Alloreactive T-Cell Profiling

Profiling of the nucleic acids expressed in alloreactive lymphocytes, such as T-cells or T-lymphocytes (“alloreactive T-cell profiling”) may also be used for diagnosing allograft rejection. Alloreactive T-cell profiling may be used alone, or in combination with genomic expression profiling, proteomic profiling or metabolomic profiling.

Alloreactive T cells are the front-line of the graft rejection immune response. They are a subset (˜0.1-1%) of the peripheral blood mononuclear cells (PBMC) which recognize allogeneic antigens present on the foreign graft. They may infiltrate the foreign graft, to initiate a cascade of anti-graft immune response, which, if unchecked, will lead to rejection and failure of the graft. Alloreactive T cells, therefore, provide specificity compared to other sources of markers, or may function as a complementary source of markers that differentiate between stages of organ rejection. Gene expression profiles from an alloreactive T cell population may further be used across different organ transplants, and may also serve to better distinguish between organ rejection and immune activation due to other reasons (allergies, viral infection and the like).

Alloreactive T-cell profiling may also be used in combination with metabolite (“metabolomics”), genomic or proteomic profiling. Minor alterations in a subject's genome, such as a single base change or polymorphism, or expression of the genome (e.g. differential gene expression) may result in rapid response in the subject's small molecule metabolite profile. Small molecule metabolites may also be rapidly responsive to environmental alterations, with significant metabolite changes becoming evident within seconds to minutes of the environmental alteration—in contrast, protein or gene expression alterations may take hours or days to become evident. The list of clinical variables indicates several metabolites that may be used to monitor, for example, cardiovascular disease, obesity or metabolic syndrome—examples include cholesterol, homocysteine, glucose, uric acid, malondialdehyde and ketone bodies. Other non-limiting examples of small molecule metabolites are listed in Table 3.

Markers from alloreactive T-cells may be used alone for the diagnosis of allograft rejection, or may be used in combination with markers from whole blood.

The present invention also provides for a core group of markers useful for the assessment, prediction or diagnosis of allograft rejection, including acute allograft rejection, comprising KLF12, TTLL5, 239901_at, 241732_at, OFD1, MIRH1, WDR21A, EFCAB2, TNRC15, LENG10, MYSM1, 237060_at, C19orf59, MCL1, ANKRD25, MYL4.

The 16 genes or transcripts (Table 9) that were detected, quantified and found to demonstrate a statistically significant fold change in the alloreactive T-cells of AR subjects relative to non-rejecting transplant (NR) controls were statistically significant in each of the moderated t-tests applied. The fold change for each marker was at least 1.6-fold, and may represent an increase/up-regulation or decrease/down-regulation of the gene or transcript in question.

A method of diagnosing acute allograft rejection in a subject as provided by the present invention comprises 1) determining the expression profile of one or more than one markers in a biological sample from the subject, the one or more than one markers selected from the group comprising KLF12, TTLL5, 239901_at, 241732_at, OFD1, MIRH1, WDR21A, EFCAB2, TNRC15, LENG10, MYSM1, 237060_at, C19orf59, MCL1, ANKRD25, MYL4; 2) comparing the expression profile of the one or more than one markers to a non-rejector allograft T-cell control profile; and 3) determining whether the expression level of the one or more than one markers is up-regulated or down-regulated relative to the control profile, wherein up-regulation or down-regulation of the markers is indicative of the rejection status.

Alloreactive T-Cell Genomic Expression Profiling Markers (“Alloreactive T-Cell Markers”)

The Kruppel-like factor 12 (KLF12) gene encodes an developmentally regulated transcription factor and has a role in vertebrate development and carcinogenesis. Nucleotide sequences of human KLF12 are known (e.g. GenBank Accession No. CH471093.1, CQ834616,1, AJ243274.1, AK291397.1).

The tubulin tyrosine ligase-like family, member 5 (TTLL5) gene encodes a protein that may have a role in catalysis of the ATP-dependent post translational modification of alpha-tubulin. Nucleotide sequences of human TTLL5 are known (e.g. GenBank Accession No. AC009399.5, AB023215.1, AK024259.1, AY237126.1).

The OFD1 (oral-facial-digital syndrome 1, 71-7A; SGBS2; CXorf5; MGC117039; MGC117040) gene is located on the X chromosome and encodes a centrosomal protein. Nucleotide sequences of human OFD1 are known (e.g. GenBank Accession No. NT_{—}011757, NM_{—}003611).

MIRH1 (microRNA host gene (non-protein coding) 1, MIRH1, C13orf25, FLJ14178, MGC126270) encodes a microRNA. Nucleotide sequences of human MIRH1 are known (e.g. GenBank Accession No. BC109081, NW_{—}001838084).

The WDR21A (WD repeat domain 21A, DKFZp434K114, MGC20547, MGC46524, WDR21) gene encodes a WD repeat-containing protein. Nucleotide sequences of human WDR21A are known (e.g. GenBank Accession No. NW_{—}001838113, NW_{—}925561n NM_{—}181340, NM_{—}181341).

The EFCAB2 gene (EF-hand calcium binding domain 2, FLJ33608, MGC12458, RP11-290P14.1) encodes a calcium ion binding protein. Nucleotide sequences of human EFCAB2 are known (e.g. GenBank Accession No. NM_{—}032328, and BC005357).

The TNRC15 (GIGYF2, GRB10 interacting GYF protein 2, PERQ2; PERQ3; FLJ23368; KIAA0642; DKFZp686115154; DKFZp686J17223) gene encodes a product that may interact with Grb10. Nucleotide sequences of human TNRC15 are known (e.g. GenBank Accession No. NW_{—}001838867, NW_{—}921618, and NT_{—}005403).

LENG10 is a leukocyte receptor cluster (LRC), member 10. Nucleotide sequences of human LENG10 is known, for example GenBank Accession No.: AF211977.

The gene for MYSM1 (myb-like, SWIRM and MPN domains 1, 2A-DUB; KIAA1915; RP4-592A1.1; DKFZp779J1554; DKFZp779J1721) encodes a deubiquitinase with a role in regulation of transcription via coordination of histone acetylation and deubiquitination. Nucleotide sequences of human MYSM1 are known, for example GenBank Accession No.: NM_{—}001085487, and NW_{—}001838579.

C19orf59 (chromosome 19 open reading frame 59, MCEMP1, MGC132456) encodes a single-pass transmembrane protein, and may have a role in regulating mast cell differentiation or immune responses. Nucleotide sequences of human C19orf59 are known, for example GenBank Accession No.: NC_{—}000019.8., and NM_{—}174918. This gene encodes

MCL1 (myeloid cell leukemia sequence 1 (BCL2-related), EAT, MCL1L, MCL1S, MGC104264, MGC1839, TM). The product encoded by this gene may be involved in regulation of apoptosis. Nucleotide sequences of human MCL1 are known, for example: GenBank Accession No.: NM_{—}021960, and NM_{—}182763.

ANKRD25 also known as KANK2 (KN motif and ankyrin repeat domains 2), DKFZp434N161, FLJ20004, KIAA1518, MGC119707, MXRA3, SIP. Nucleotide sequences of human MCL1 are known, for example: GenBank Accession No.: NM_{—}015493, AB284125, and DJ053242. The product of the ANKRD25 gene may be an SRC interacting protein (SIP) and have a role in sequestering SRC coactivators in the cytoplasm and buffer the availability of these coactivators, thus providing a mechanism for the regulation of the transcription regulators.

MYL4 (myosin, light chain 4, alkali; atrial, embryonic), also known as ALC1, AMLC, GT1, and PRO1957. Nucleotide sequences of human MYL4 are known, for example: GenBank Accession No.: NM_{—}000258, NW_{—}001838448, NW_{—}926883, NM_{—}001002841 and NM_{—}002476. The product encoded by this gene encodes a myosin alkali light chain that is found in embryonic muscle and adult atria.

The invention also provides for a kit for use in predicting or diagnosing a subject's rejection status. The kit may comprise reagents for specific and quantitative detection of KLF12, TTLL5, 239901_at, 241732_at, OFD1, MIRH1, WDR21A, EFCAB2, TNRC15, LENG10, MYSM1, 237060_at, C19orf59, MCL1, ANKRD25, MYL4, along with instructions for the use of such reagents and methods for analyzing the resulting data. The kit may be used alone for predicting or diagnosing a subject's rejection status, or it may be used in conjunction with other methods for determining clinical variables, or other assays that may be deemed appropriate. The kit may include, for example, one or more labelled oligonucleotides capable of selectively hybridizing to the marker. The kit may further include, for example, one or more oligonucleotides operable to amplify a region of the marker (e.g. by PCR). Instructions or other information useful to combine the kit results with those of other assays to provide a non-rejection cutoff index for the prediction or diagnosis of a subject's rejection status may also be provided.

Methods for selecting and manufacturing such oligonucleotides, as well as their inclusion on a ‘chip’ or an array, and methods of using such chips or arrays are referenced or described herein.

### Proteomic Profiling for Diagnosing Allograft Rejection

Proteomic profiling may also be used for diagnosing allograft rejection. Proteomic profiling may be used alone, or in combination with genomic expression profiling, metabolite profiling, or alloreactive T-cell profiling.

In some embodiments, the invention provides for a method of diagnosing acute allograft rejection in a subject comprising 1) determining the expression profile of one or more than one proteomic markers in a biological sample from the subject, the proteomic markers selected from the group comprising a polypeptide encoded by B2M, F10, CP, CST3, ECMP1, CFH, C1QC, CFI, APCS, C1R, SERPINF1, PLTP, ADIPOQ and SHBG; 2) comparing the expression profile of the one or more than one proteomic markers to a non-rejector profile; and 3) determining whether the expression level of the one or more than one proteomic markers is increased or decreased relative to the control profile, wherein increase or decrease of the one or more than one proteomic markers is indicative of the acute rejection status.

The invention also provides for a method of predicting, assessing or diagnosing allograft rejection in a subject as provided by the present invention comprises 1) measuring the increase or decrease of five or more than five proteomic markers selected from the group comprising a polypeptide encoded by B2M, F10, CP, CST3, ECMP1, CFH, C1QC, CFI, APCS, C1R, SERPINF1, PLTP, ADIPOQ and SHBG; and 2) determining the ‘rejection status’ of the subject, wherein the determination of ‘rejection status’ of the subject is based on comparison of the subject's proteomic marker expression profile to a control proteomic marker expression profile. The five or more than five markers may include a polypeptide encoded by PLTP, ADIPOQ, B2M, F10 and CP. In some embodiments of the invention, the five or more than five markers include a polypeptide encoded by PLTP, ADIPOQ, B2M, F10 and CP, and one or more than one of ECMP1, C1QC, C1R and SERPINF1.

A myriad of non-labelling methods for protein identification and quantitation are currently available, such as glycopeptide capture (Zhang et al., 2005. Mol Cell Proteomics 4:144-155), multidimensional protein identification technology (Mud-PIT) Washburn et al., 2001 Nature Biotechnology (19:242-247), and surface-enhanced laser desorption ionization (SELDI-TOF) (Hutches et al., 1993. Rapid Commun Mass Spec 7:576-580). In addition, several isotope labelling methods which allow quantification of multiple protein samples, such as isobaric tags for relative and absolute protein quantification (iTRAQ) (Ross et al, 2004 Mol Cell Proteomics 3:1154-1169); isotope coded affinity tags (ICAT) (Gygi et al., 1999 Nature Biotecnology 17:994-999), isotope coded protein labelling (ICPL) (Schmidt et al., 2004. Proteomics 5:4-15), and N-terminal isotope tagging (NIT) (Fedjaev et al., 2007 Rapid Commun Mass Spectrom 21:2671-2679; Nam et al., 2005. J Chromatogr B Analyt Technol Biomed Life Sci. 826:91-107), have become increasingly popular due to their high-throughput performance, a trait particular useful in biomarker screening/identification studies.

A multiplexed iTRAQ methodology was employed for identification of plasma proteomic markers in allograft recipients. iTRAQ was first described by Ross et al, 2004 (Mol Cell Proteomics 3:1154-1169). Briefly, subject plasma samples (control and allograft recipient) were depleted of the 14 most abundant proteins and quantitatively analyzed by iTRAQ-MALDI-TOF/TOF. resulted in the identification of about 200 medium-to-low abundant proteins per iTRAQ run and 1000 proteins cumulatively. Of these, 129 of proteins were detected in at least ⅔ of samples within AR and NR groups, and were considered for statistical analyses. Fourteen candidate plasma proteins with differential relative concentrations between AR and NR were identified. Two classifiers were constructed using LDA, a multivariate analysis that seeks for the linear combination of markers that best discriminates both groups. Results were validated further using additional samples (test set) from an extended cohort of patients. (A technical validation using ELISA was also performed and corroborated the results from iTRAQ. The ELISA results on their own demonstrated differential protein levels in AR versus NR samples.

Thus, although single candidate biomarkers may not clearly differentiate groups (with some fold-changes being relatively small), together, the identified markers achieved a satisfactory classification (100% sensitivity and >91% specificity).

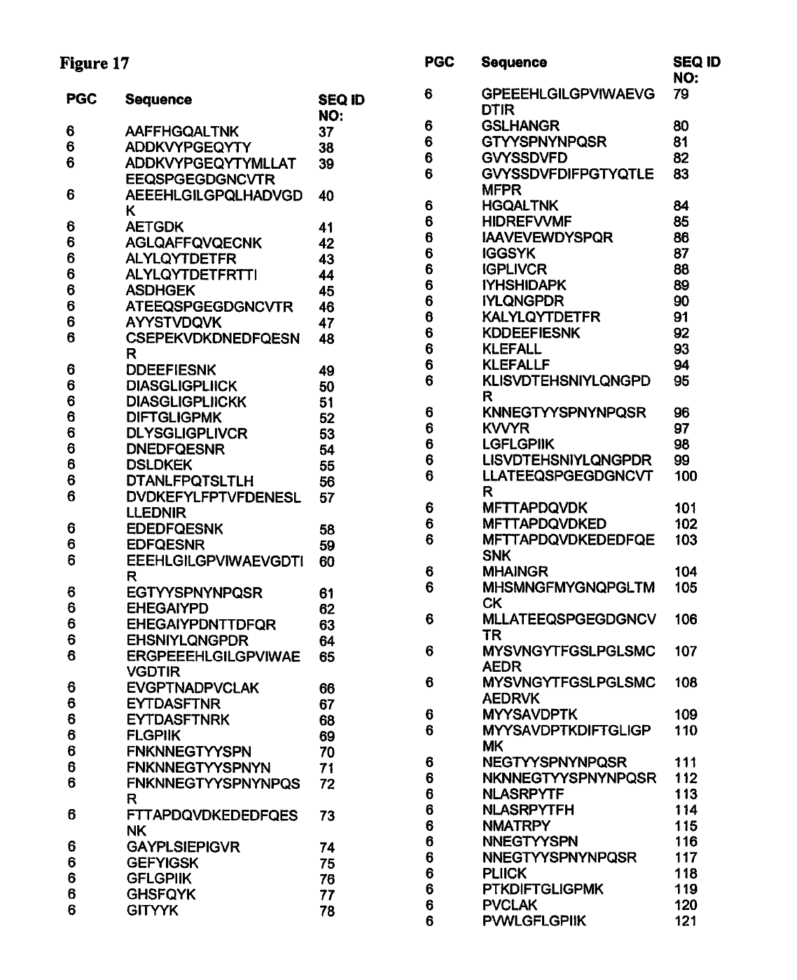

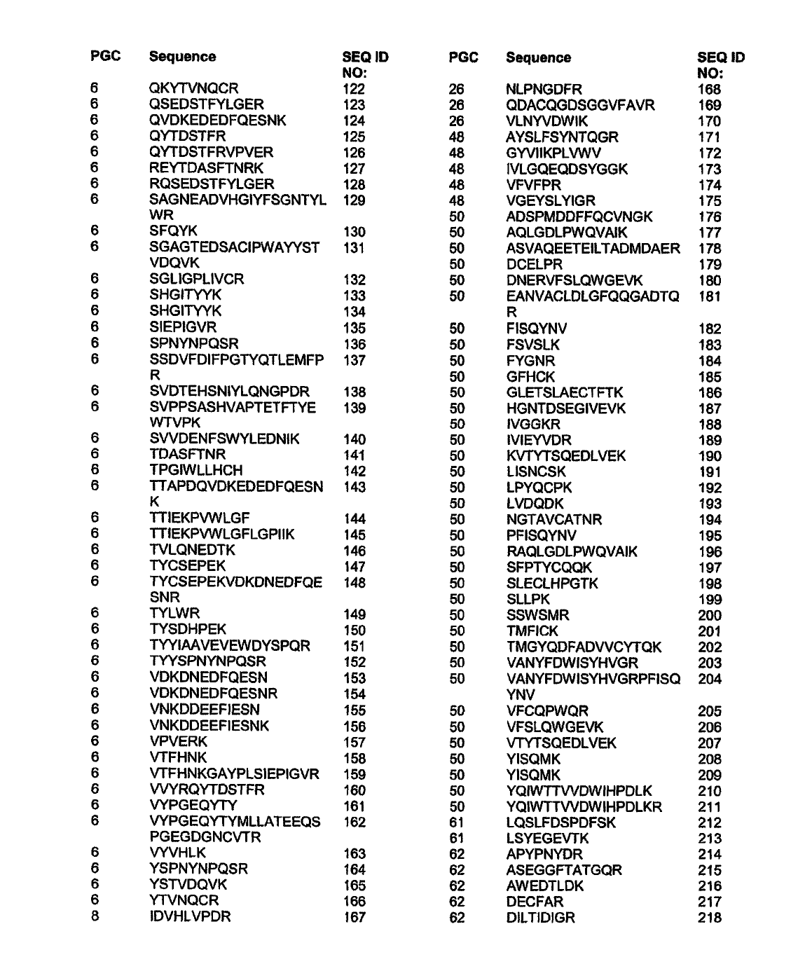

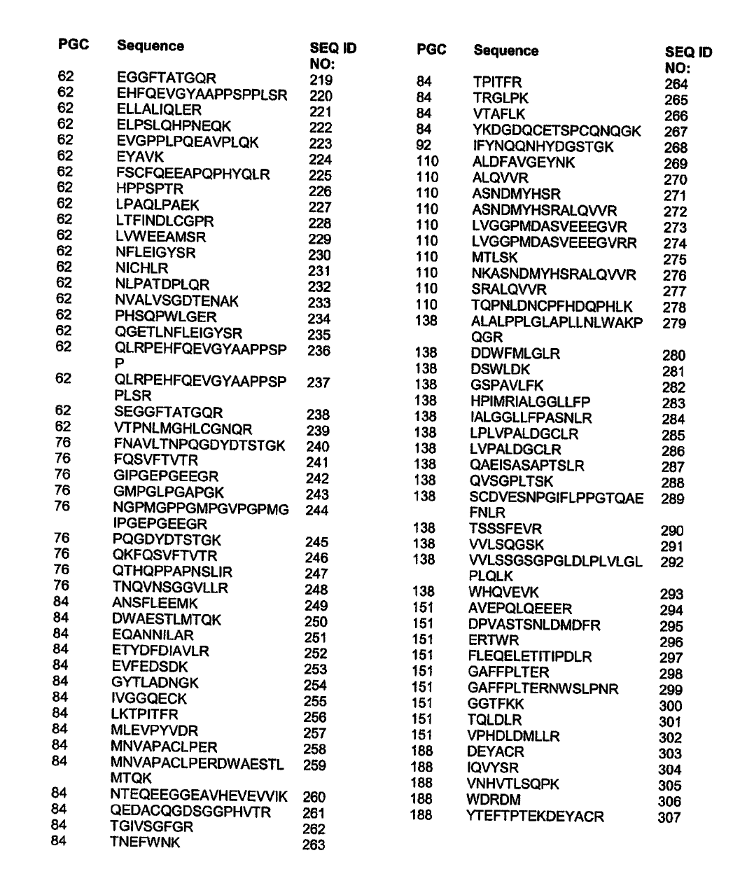

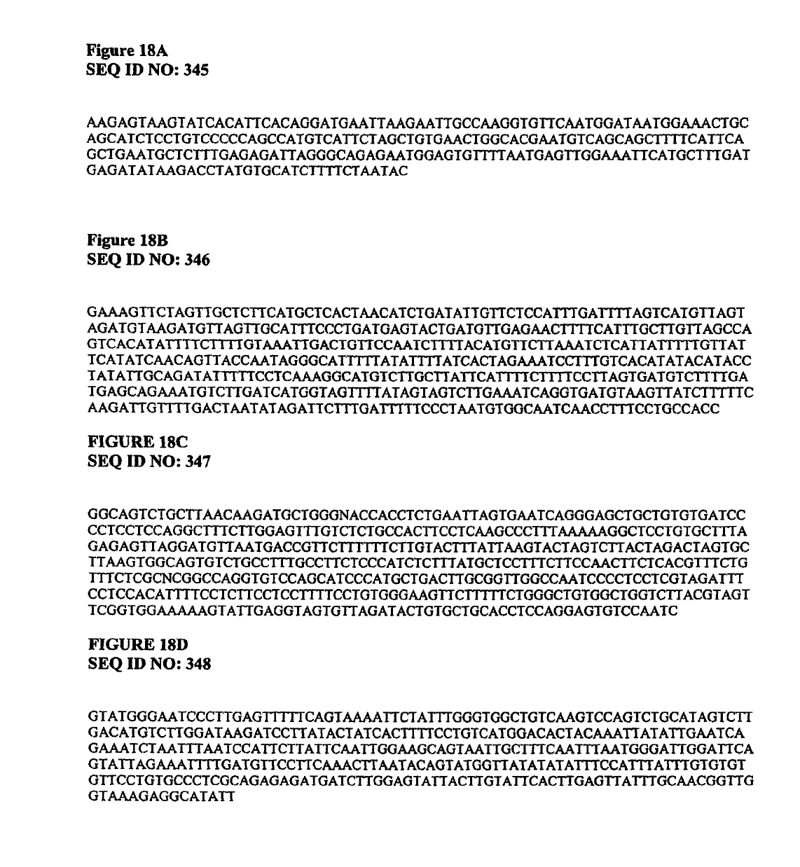

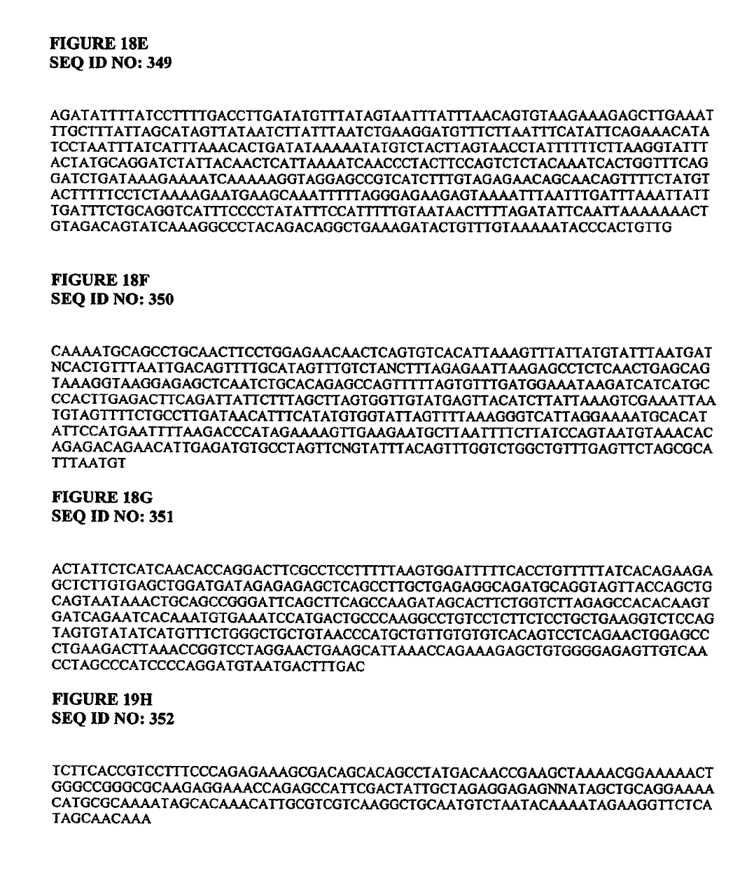

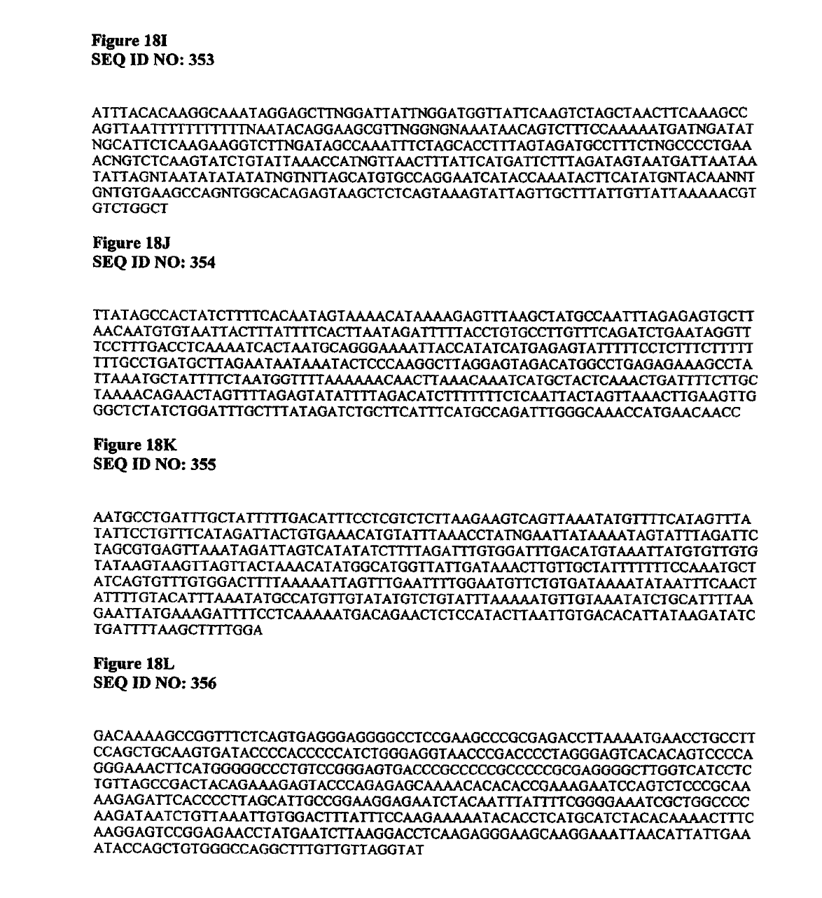

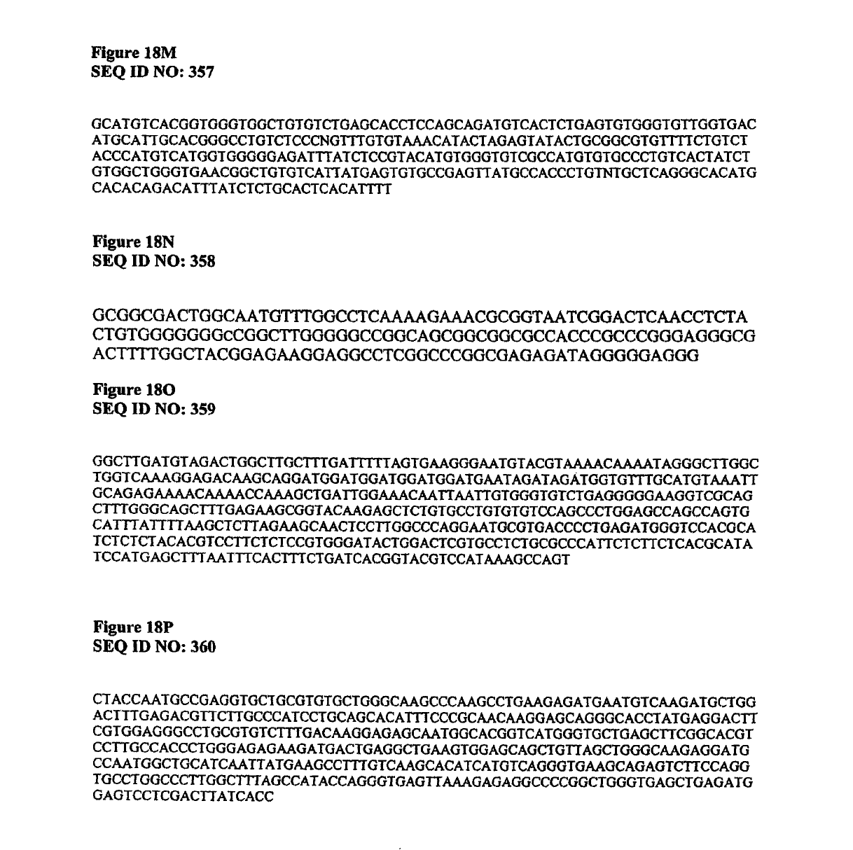

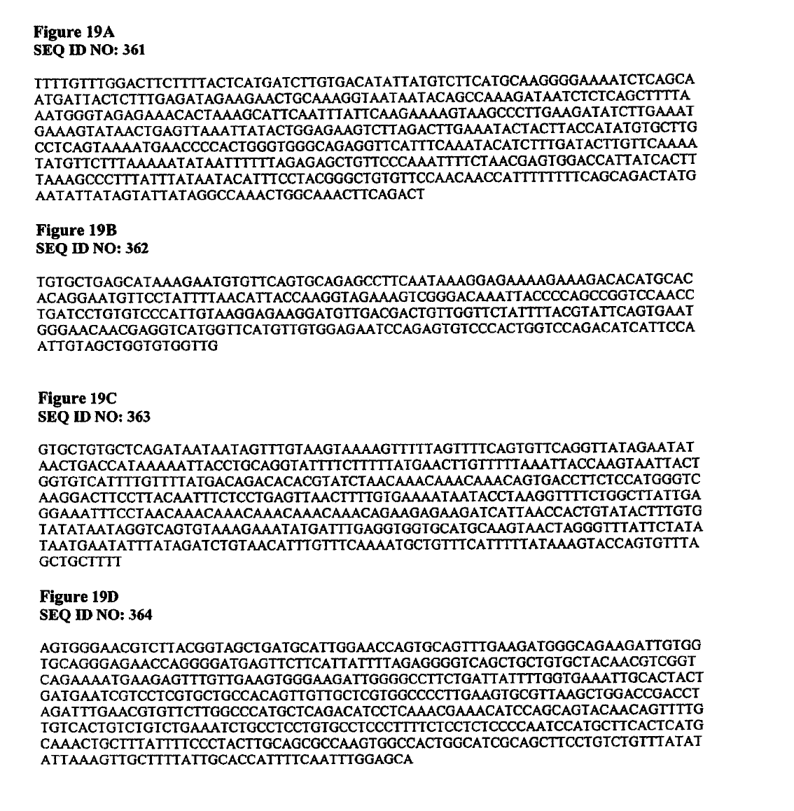

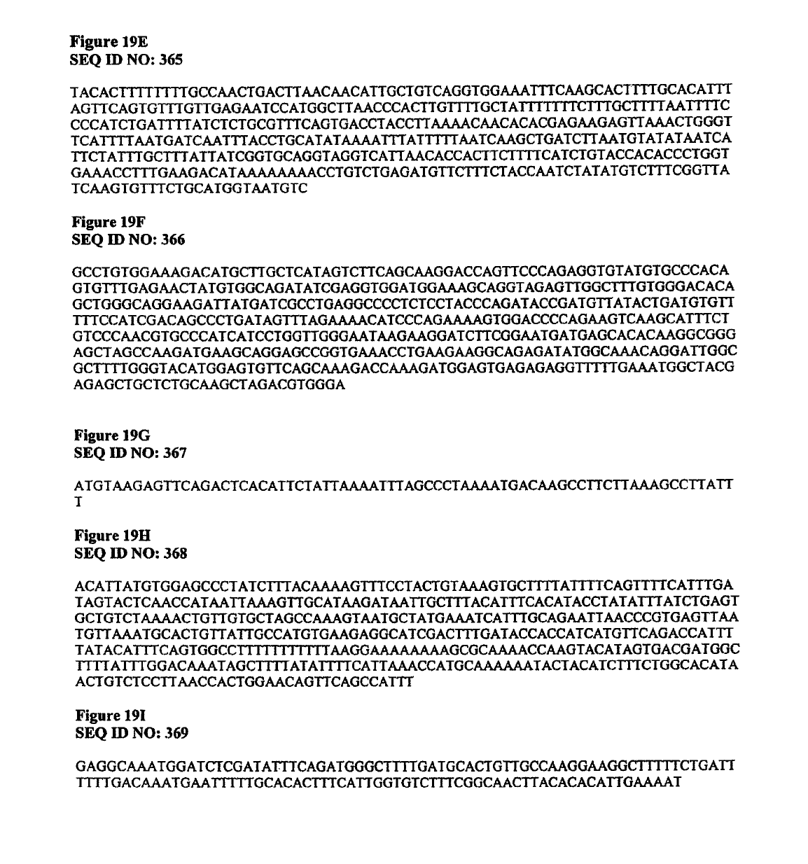

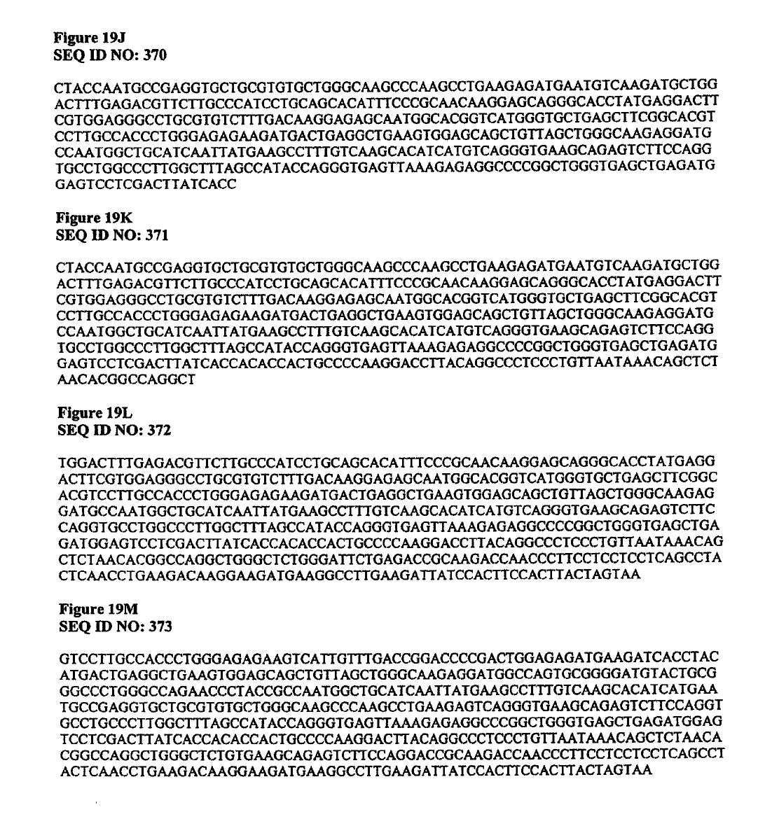

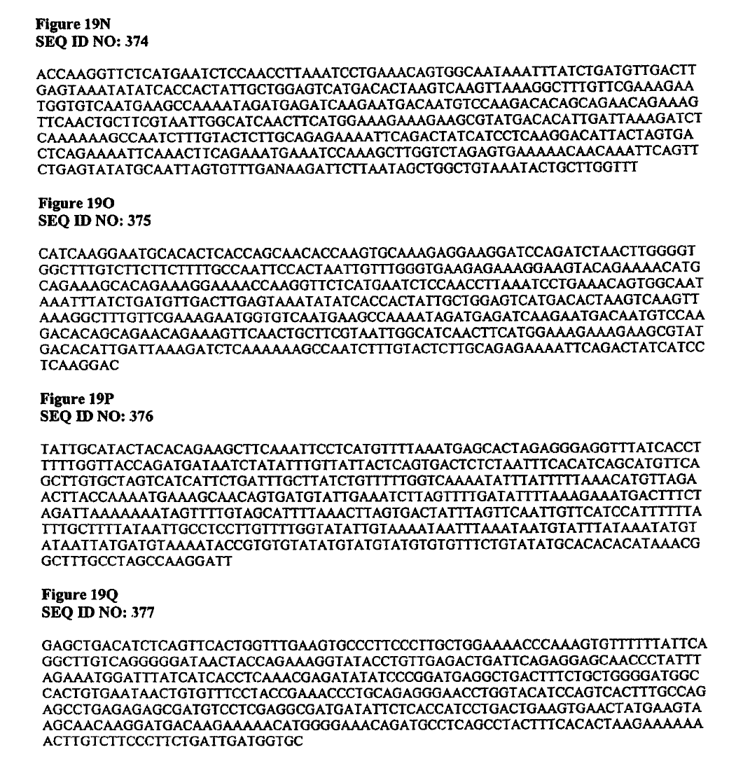

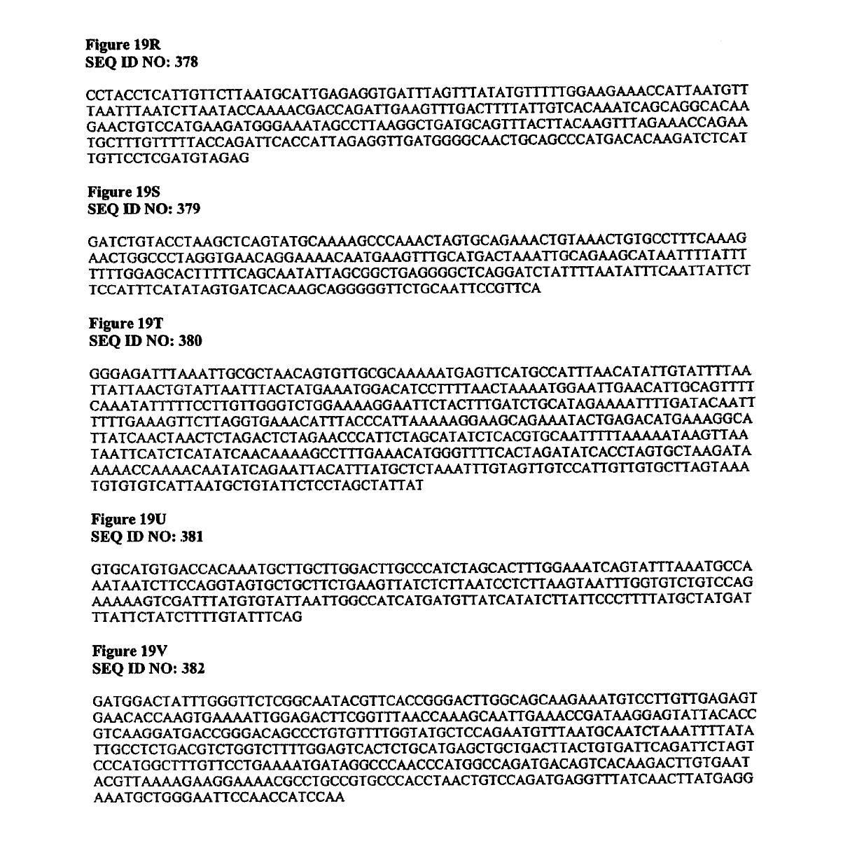

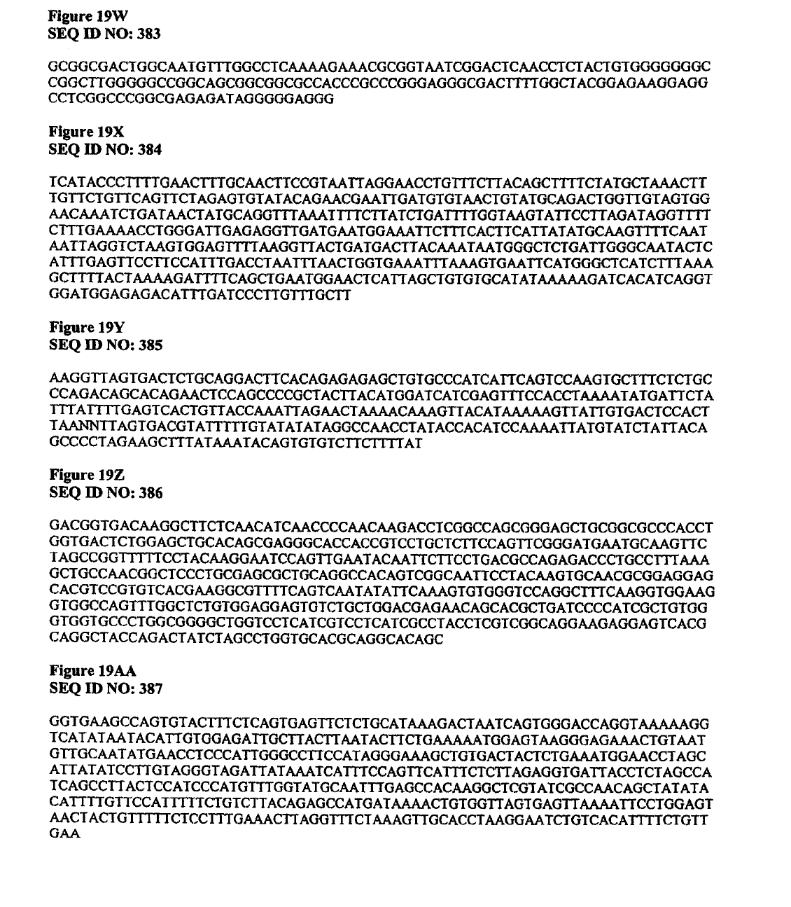

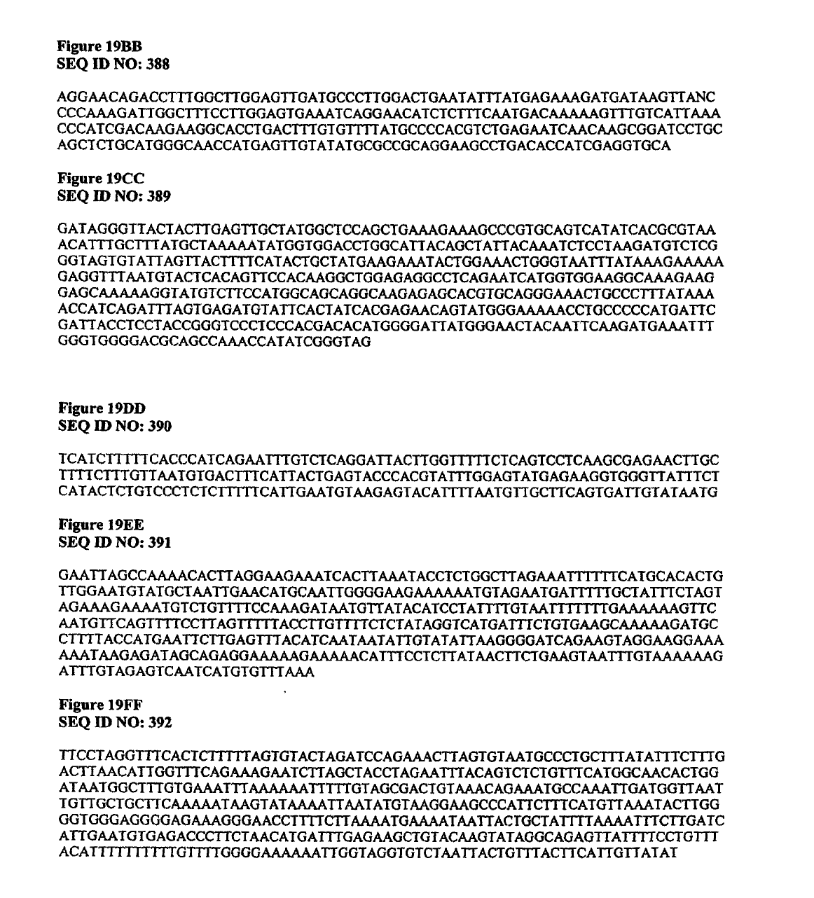

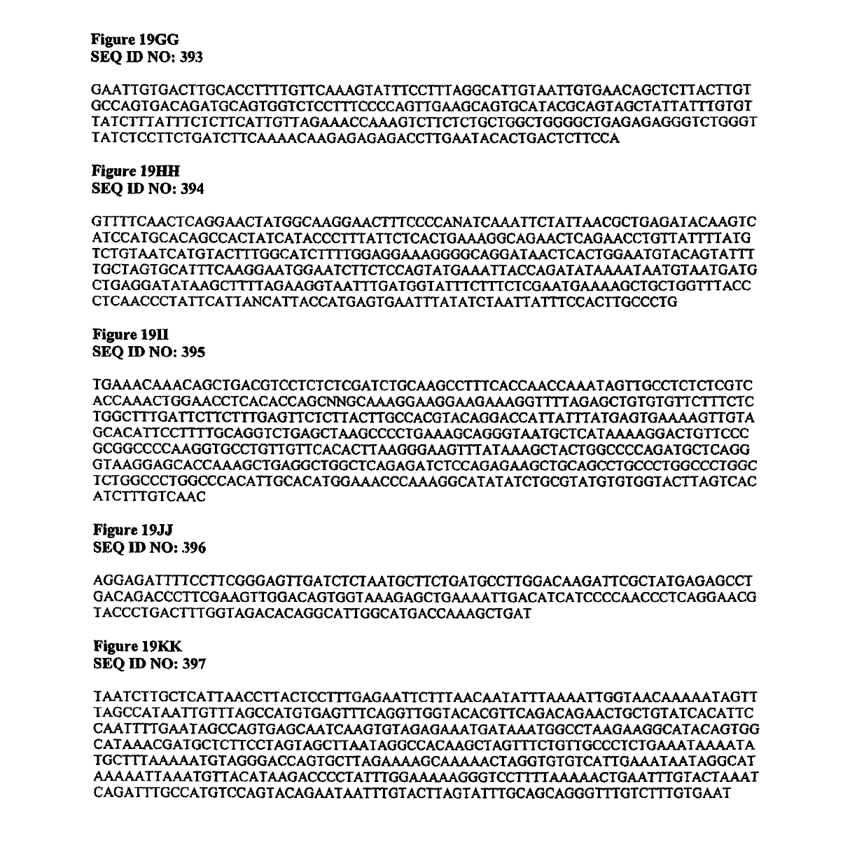

Exemplary peptide sequences comprising one or more proteomic markers that may be detected in a sample are provided in FIG. 17. These peptides were produced by a tryptic digest (as described herein) and identified in the iTRAQ experiments. Detection of one or more than one peptide in a sample is indicative of the proteomic marker being present in the sample. While iTRAQ was one exemplary method used to detect the peptides, other methods described herein, for example immunological based methods such as ELISA may also be useful. Alternately, specific antibodies may be raised against the one or more proteins, isoforms, precursors, polypeptides, peptides, or portions or, fragments thereof, and the specific antibody used to detect the presence of the one or more proteomic marker in the sample. Methods of selecting suitable peptides, immunizing animals (e.g. mice, rabbits or the like) for the production of antisera and/or production and screening of hybridomas for production of monoclonal antibodies are known in the art, and described in the references disclosed herein.

Proteomic Expression Profiling Markers (“Proteomic Markers”)

One or more precursors, splice variants, isoforms may be encoded by a single gene Examples of genes and the isoforms, precursors and variants encoded are provided in Table 8, under the respective Protein Group Code (PGC).

A polypeptide encoded by PLTP (isoform 1) (Phospholipid Transfer Protein; alternately referred to as Lipid transfer protein II, HDLCQ9) is a lipid transfer protein in human serum, and may have a role in high density lipoprotein (HDL) remodeling and cholesterol metabolism. Nucleotide sequences encoding PLTP are known (e.g. GenBank Accession Nos. AY509570, NM_{—}006227, NM_{—}182676). Amino acid sequences for PLTP are known (e.g. GenPept Accession Nos AAA36443, NP_{—}872617, NP_{—}006218, P55058).

A polypeptide encoded by ADIPOQ (Adiponectin; alternately referred to as APM1, ADPN, Adipocyte, C1q-, and collagen domain-containing, ACRP30) is a hormone secreted by adipocytes that regulates energy homeostasis and glucose and lipid metabolism. Nucleotide sequences encoding ADIPOQ are known (e.g. GenBank Accession No. EU420013, BC096308, NM_{—}004797). Amino acid sequences for ADOPOQ are known (e.g. GenPept Accession No. NP_{—}004788, CAB52413, Q60994, Q15848, BAA08227).

A polypeptide encoded by B2M (Beta-2-Microglobulin) is a serum protein found in association with the major histocompatibility complex (MHC) class 1 heavy chain on the surface of most nucleated cells. Nucleotide sequences encoding B2M are known (e.g. GenBank Accession No. NM_{—}004048, BU658737.1, BC032589.1 and AI686916.1.). Amino acid sequences for B2M are known (e.g. GenPept Accession No. P61769, AAA51811, CAA23830).

A polypeptide encoded by F10 (Coagulation Faxtor X, Factor X) is the zymogen of factor Xa, a serine protease that occupies a pivotal position in the clotting process. It is activated either by the contact-activated (intrinsic) pathway or by the tissue factor (extrinsic) pathway. Factor Xa, in combination with factor V, then activates prothrombin to form the effector enzyme of the coagulation cascade Nucleotide sequences encoding F10 are known (e.g. GenBank Accession No. NG_{—}009258, NM_{—}000504, CB158437.1, CR607773.1 and BC046125.1.). Amino acid sequences for F10 are known (e.g. AAA52490, AAA527644, AAA52486, P00742).

A polypeptide encoded by CP (Ceruloplasmin, also known as ferroxidase; iron (II):oxygen oxidoreductase, EC 1.16.3.1) is a blue alpha-2-glycoprotein that binds 90 to 95% of plasma copper and has 6 or 7 cupric ions per molecule. It is involved in peroxidation of Fe(II) transferrin to form Fe(III) transferrin. CP is a plasma metalloprotein. Nucleotide sequences encoding CP are known (e.g. GenBank Accession No. NG_{—}001106, NM_{—}000096, DC334592.1, BC142714.1 and BC146801.1). Amino acid sequences for CP are known (e.g. GenPept Accession No. NP_{—}000087, DC334592.1, BC142714.1 and BC146801.1).

A polypeptide encoded by ECMP1 (ECM1, Extracellular Matrix Protein 1) is expressed in many tissue types and associates with connective tissue proteins and has been demonstrated to promote angiogenesis and play a role in endothelial cell proliferation, wound repair and matrix remodeling. ECM1 is involved in the wnt/β-catenin signaling pathway. Nucleotide sequences encoding ECMP1 are known (e.g. GenBank Accession No. NM_{—}022664, NM_{—}004425, DA963826.1, U68186.1, CR593353.1 and CA413352.1.). Amino acid sequences for ECMP1 are known (e.g. GenPept Accession No. NP_{—}073155, NP_{—}004416, AAB88082, AAB88081).

A polypeptide encoded by C1QC (Complement component C1q, C chain) is a component of complement C1, an initiator of the classical complement pathway. Nucleotide sequences encoding CIQC are known (e.g. GenBank Accession No. NM_{—}172369, NM_{—}001114101, CB995661.1, DA849505.1, BC009016.1 and BG060138.1). Amino acid sequences for C1QC are known (e.g. GenPept Accession No. NP_{—}001107573, NP_{—}758957, P02747).

A polypeptide encoded by C1R (Complement component 1, r subcomponent) is part of a complex including C1q, C1r and C1s to form the complement protein C1. Nucleotide sequences encoding C1R are known (e.g. GenBank Accession No. NM_{—}001733, BC035220.1.). Amino acid sequences for C1R are known (e.g. GenPept Accession No. P00736, NP_{—}001724, AAA58151, CAA28407).

A polypeptide encoded by SERPINF1 (PEDF, Pigment Epithelium-derived factor) is a serine protease inhibitor. Nucleotide sequences encoding SERPINF1 are known (e.g. GenBank Accession No. NM_{—}002615, AA351026.1, CA405781.1, BU154385.1, BM981180.1, BQ773314.1, W22661.1 and AA658568.1.). Amino acid sequences for SERPINF1 are known (e.g. GenPept Accession No. NP_{—}002606, P36955, AAA60058).

A polypeptide encoded by CST3 (Cystatin 3, cystatin C, Gamma-trace) is an inhibitor of lysosomal proteinases. Nucleotide sequences encoding CST3 are known (e.g. GenBank Accession No. NM_{—}000099, BC13083.1). Amino acid sequences for CST3 are known (e.g. GenPept Accession No. NP_{—}000090, CAG46785.1, CAA29096.1).

A polypeptide encoded by SHBG (Sex-hormone binding globulin, androgen-binding protein, ABP, testosterone-binding beta-globulin, TEBG) is a plasma glycoprotein that binds sex steroids. Nucleotide sequences encoding SHBG are known (e.g. GenBank Accession No. AK302603.1, NM_{—}001040.2). Amino acid sequences for SHBG are known (e.g. GenPept Accession No. P04728.2, CAA34400.1, NP001031.2).

A polypeptide encoded by CFH (Complement factor H, FH) is secreted into the bloodstream and has an essential role in the regulation of complement activation. Nucleotide sequences encoding CFH are known (e.g. GenBank Accession No. NM_{—}000186.3, NM001014975.2, BM842566.1, Y00716.1, AL049744.8, BP324193.1 and BC142699.1.). Amino acid sequences for CFH are known (e.g. GenPept Accession No. NP_{—}000177.2, NP_{—}001014975.1, P08603.4, Q14006, Q5TFM2).

A polypeptide encoded by CFI (Complement component I (“eye”), Complement factor I, C3b inactivator) is a serine proteinase in the complement pathway responsible for cleaving and inactivating the activities of C4b and C3b. Nucleotide sequences encoding CFI are known (e.g. GenBank Accession No. NM_{—}000204, DC392360.1, J02770.1, AK290625.1, N63668.1 and BM955734.1.). Amino acid sequences for CFI are known (e.g. GenPept Accession No. NP_{—}000195, P05156, AAA52466).

A polypeptide encoded by APCS (Amyloid P component, serum; Serum amyloid P, SAP) is a member of the pentraxin family, and a constituent of amyloid protein deposits Nucleotide sequences encoding APCS are known (e.g. GenBank Accession No. NM_{—}001639, CR450313, BC070178). Amino acid sequences for APCS are known (e.g. GenPept Accession No. NP_{—}001630, P02743, AAA60302, BAA00060).

Interpretation of the large body of expression data obtained from, for example, iTRAQ protein or proteomic experiments, but is greatly facilitated through use of algorithms and statistical tools designed to organize the data in a way that highlights systematic features. Visualization tools are also of value to represent differential expression by, for example, varying intensity and hue of colour. The algorithm and statistical tools available have increased in sophistication with the increase in complexity of arrays and the resulting datasets, and with the increase in processing speed, computer memory, and the relative decrease in cost of these.

Mathematical and statistical analysis of protein or polypeptide expression profiles may accomplish several things—identification of groups of genes that demonstrate coordinate regulation in a pathway or a domain of a biological system, identification of similarities and differences between two or more biological samples, identification of features of a gene expression profile that differentiate between specific events or processes in a subject, or the like. This may include assessing the efficacy of a therapeutic regimen or a change in a therapeutic regimen, monitoring or detecting the development of a particular pathology, differentiating between two otherwise clinically similar (or almost identical) pathologies, or the like.

The pattern of protein or polypeptide expression in a biological sample may also provide a distinctive and accessible molecular picture of its functional state and identity (DeRisi 1997; Cho 1998; Chu 1998; Holstege 1998; Spellman 1998). Two different samples that have related gene expression patterns are therefore likely to be biologically and functionally similar to one another, conversely two samples that demonstrate significant differences may not only be differentiated by the complex expression pattern displayed, but may indicate a diagnostic subset of gene products or transcripts that are indicative of a specific pathological state or other physiological condition, such as allograft rejection.

The present invention provides for a core group of markers useful for the assessment, prediction or diagnosis of allograft rejection, including acute allograft rejection, comprising five or more than five of B2M, F10, CP, CST3, ECMP1, CFH, C1QC, CFI, APCS, C1R, SERPINF1, PLTP, ADIPOQ and SHBG.

The invention also provides for a kit for use in predicting or diagnosing a subject's rejection status. The kit may comprise reagents for specific and quantitative detection of five or more than five of B2M, F10, CP, CST3, ECMP1, CFH, C1QC, CFI, APCS, C1R, SERPINF1, PLTP, ADIPOQ and SHBG, along with instructions for the use of such reagents and methods for analyzing the resulting data. For example, the kit may comprise antibodies or fragments thereof, specific for the proteomic markers (primary antibodies), along with one or more secondary antibodies that may incorporate a detectable label; such antibodies may be used in an assay such as an ELISA. Alternately, the antibodies or fragments thereof may be fixed to a solid surface, e.g. an antibody array. The kit may be used alone for predicting or diagnosing a subject's rejection status, or it may be used in conjunction with other methods for determining clinical variables, or other assays that may be deemed appropriate. Instructions or other information useful to combine the kit results with those of other assays to provide a non-rejection cutoff index for the prediction or diagnosis of a subject's rejection status may also be provided.

Methods for selecting and manufacturing such antibodies, as well as their inclusion on a ‘chip’ or an array, or in an assay, and methods of using such chips, arrays or assays are referenced or described herein.

### Metabolite Profiling for Diagnosing Allograft Rejection

Metabolite profiling (“metabolomics” or “metabolomic profiling”) may also be used for diagnosing allograft rejection. Metabolite profiling may be used alone, or in combination with genomic expression profiling, proteomic profiling or alloreactive T-cell profiling. Minor alterations in a subject's genome, such as a single base change or polymorphism, or expression of the genome (e.g. differential gene expression) may result in rapid response in the subject's small molecule metabolite profile. Small molecule metabolites may also be rapidly responsive to environmental alterations, with significant metabolite changes becoming evident within seconds to minutes of the environmental alteration—in contrast, protein or gene expression alterations may take hours or days to become evident. The list of clinical variables indicates several metabolites that may be used to monitor, for example, cardiovascular disease, obesity or metabolic syndrome—examples include cholesterol, homocysteine, glucose, uric acid, malondialdehyde and ketone bodies.

Of a set of 33 metabolites (Table 3) that were detected and quantified in a population of AR subjects and NR subjects, 5 demonstrated a statistically significant change in the AR subjects compared to NR subjects. The fold-change varied depending on the marker and the comparison method used—a fold-change of at least 0.44 for taurine (decrease), 0.59 for serine (decrease) and 0.75 for glycine (decrease) using an absolute concentration based analysis; or a fold change of at least 0.65 for glycine (decrease), 2.9 for creatine (increase) and 1.89 (increase) for carnitine. The balance of the metabolites did not exhibit a statistically significant change compared to the NR subject population.

Metabolomic Expression Profiling Markers (“Metabolomic Markers” or “Metabolic Markers”)

Creatine (2-(carbamimidoyl-methyl-amino)acetic acid; CAS Registry No. 57-00-1) is an amino acid found in various tissues—in muscle tissue it is found in a phosphorylated form (phosphocreatine). Creatine is involved in ATP metabolism for cellular energy, and is excreted in the urine as creatinine. The high energy phosphate group of ATP is transferred to creatine to form phosphocreatine—this is reversibly catalyzed by creatine kinase.

Taurine (2-amino-Ethanesulfonic acid; CAS Registry No. 107-35-7) is a sulfur-containing amino acid. It is an essential amino acid in pre-term and newborns in humans and other species. Taurine has multiple roles in the body, including neurotransmitter, cell membrane stabilization and ion transport. Decreased myocardial taurine level has been previously found to be associated with ischemic heart failure (Kramer et al 1981 Am. J. Physiol. 240:H238-46).

Carnitine ((L-)carnitine; (3R)-3-hydroxy-4-trimethylammonio-butanoate; CAS Registry No. 541-15-1) is a nitrogen-containing amino acid, and can be synthesized by most healthy organisms. It also has a key role in energy metabolism (specifically fatty acid transport in the mitochondria) in muscles.

Glycine (2-amioacetic acid; CAS Registry No. 56-40-6) is a nonessential amino acid involved in production of various important biopolymers (protein, nucleic acid, collagen, phospholipids) and also in energy release.

Serine ((L-) serine; 2-amino-3-hydroxy-propanoic acid; CAS Registry No. 56-45-1) is a nonessential amino acid derived from glycine. Serine may exhibit concentration in cell membranes, and products of its metabolism may be essential for cell proliferation and also for specific functions in the CNS—L-serine is a carbon source for de novo synthesis of purine nucleotides, and deoxythymidine monophosphate. In recent years, L-serine and the products of its metabolism have been recognized not only to be essential for cell proliferation, but also to be necessary for specific functions in the central nervous system (e.g. De Konig et al. 2003. Biochem J. 371:653-61). Without wishing to be bound by theory, given that serine may be derived from glycine, the relative lower level of serine observed in AR patients may be in line with the experimental results observed for glycine.

TABLE 3
Metabolites identified and quantified in NMR spectra
of serum samples obtained from subject population.
Compound Name

| | |
|---|---|
| Glucose | Lactate |
| Glutamine | Alanine |
| Glycine | Proline |
| Glycerol | Valine |
| Taurine | Lysine |
| Citrate | Serine |
| Leucine | Ornithine |
| Creatinine | Tyrosine |
| Phenylalanine | Pyruvate |
| Histidine | Carnitine |
| Glutamate | Acetate |
| Isoleucine | Asparagine |
| Betaine | 3-Hydroxybutyrate |
| Creatine | Propylene glycol |
| 2-Hydroxybutyrate | Formate |
| Methionine | Choline |
| Acetone | |

Therefore, a method for diagnosing allograft rejection in a subject as provided by the present invention comprises 1) measuring the concentration of at least three markers selected from the group comprising serine, glycine, taurine, creatine or carnitine; 2) comparing the concentration of each of the at least three markers to a non-rejector cutoff index, and 3) determining the ‘rejection status’ of the subject; whereby the rejection status of the subject is indicated by the concentration of each of the at least three markers being above or below the non-rejector cutoff index.

Various techniques and methods may be used for obtaining a metabolite profile of a subject. The particulars of sample preparation may vary with the method used, and also on the metabolites of interest—for example, to obtain a metabolite profile of amino acids and small, generally water soluble molecules in the sample may involve filtration of the sample with a low molecular weight cutoff of 2-10 kDa, while obtaining a metabolite profile of lipids, fatty acids and other generally poorly-water soluble molecules may involve one or more steps of extraction with an organic solvent and/or drying and resolubilization of the residues. While some exemplary methods of detecting and/or quantifying markers have been indicated herein, others will be known to those skilled in the art and readily usable in the methods and uses described in this application.

Some examples of techniques and methods that may be used (either singly or in combination) to obtain a metabolite profile of a subject include, but are not limited to, nuclear magnetic resonance (NMR), gas chromatography (GC), gas chromatography in combination with mass spectroscopy (GC-MS), mass spectroscopy, Fourier transform MS (FT-MS), high performance liquid chromatography or the like. Exemplary methods for sample preparation and techniques for obtaining a metabolite profile may be found at, for example, the Human Metabolome Project website (Wishart D S et al., 2007. Nucleic Acids Research 35:D521-6).

Standard reference works setting forth the general principles of such methods useful in metabolite profiling as would be known to those of skill in the art include, for example, Handbook of Pharmaceutical Biotechnology, (ed. SC Gad) John Wiley & Sons, Inc., Hoboken, N.J., (2007), Chromatographic Methods in Clinical Chemistry and Toxicology (R Bertholf and R. Winecker, eds.) John Wiley & Sons, Inc., Hoboken, N.J., (2007), Basic One-and Two-Dimensional NMR Spectroscopy by H., Friebolin. Wiley-VCH 4^{th} Edition (2005).

In one example, at least three markers are selected from the group comprising creatine, taurine, serine, carnitine, glycine. Quantification of the markers in the biological sample may be determined by any of several methods known in the art. Concentration of the markers may be determined as an absolute value, or relative to a baseline value, and the concentration of the subject's markers compared to a cutoff index (e.g. a non-rejection cutoff index).

Access to the methods of the invention may be provided to an end user by, for example, a clinical laboratory or other testing facility performing the individual marker tests—the biological samples are provided to the facility where the individual tests and analyses are performed and the predictive method applied; alternately, a medical practitioner may receive the marker values from a clinical laboratory and use a local implementation or an internet-based implementation to access the predictive methods of the invention.

The invention also provides for a kit for use in predicting or diagnosing a subject's rejection status. The kit may comprise reagents for specific and quantitative detection of taurine, glycine, carnitine, creatine or serine, along with instructions for the use of such reagents and methods for analyzing the resulting data. The kit may be used alone for predicting or diagnosing a subject's rejection status, or it may be used in conjunction with other methods for determining clinical variables, or other assays that may be deemed appropriate. Instructions or other information useful to combine the kit results with those of other assays to provide a non-rejection cutoff index for the prediction or diagnosis of a subject's rejection status may also be provided.

### Methods

### Subjects and Specimens for Genomic, Metabolomic and Alloreactive T-Cell Genomic Studies

Subjects were enrolled according to Biomarkers in Transplantation standard operating procedures. Subjects waiting for a cardiac transplant at the St. Paul's Hospital, Vancouver, BC were approached by the research coordinators and 39 subjects who consented were enrolled in the study. All cardiac transplants are overseen by the British Columbia Transplant (BCT) and all subjects receive standard immunosuppressive therapy. Age, gender, ethnicity and primary disease of the subjects are summarized in Table 4, below. Blood samples from consented subjects were taken before transplant (baseline) and at weeks 1, 2, 3, 4, 8, 12, 26 and every 6 months up to 3 years post-transplant. Blood was collected in PAXGene™ tubes, placed in an ice bath for delivery, frozen at −20° C. for one day and then stored at −80° C. until RNA extraction.

TABLE 4
Cardiac transplant subject demographics.

| | Subjects with | Subjects without |
|---|---|---|
| | AR (n = 6) | AR (n = 12) |
| Mean Age (standard deviation | 48.73 (16.64) | 54.32 (14.83) |
| Gender (n, % male) | 4 (66.6%) | 10 (83.4%) |
| Ethnicity (n, %) | | |
| Caucasian | 6 (100%) | 10 (83.4%) |
| Filipino | — | 1 (8.3%) |
| Other | — | 1 (8.3%) |
| Primary Disease (n, %) | | |
| Cardiomyopathy - Ischemic | 4 (66.6%) | 5 (41.7%) |
| (coronary artery disease) | | |
| Cardiomyopathy - Idiopathic | 1 (16.7%) | 2 (16.7%) |
| dilated | | |
| Cardiomyopathy - Dilated | 1 (16.7%) | 1 (8.3%) |
| Cardiomyopathy - Unspecified | — | 2 16.7%) |
| Congenital heart disease | — | 1 (8.3%) |
| Cardiogenic shock | — | 1 (8.3%) |

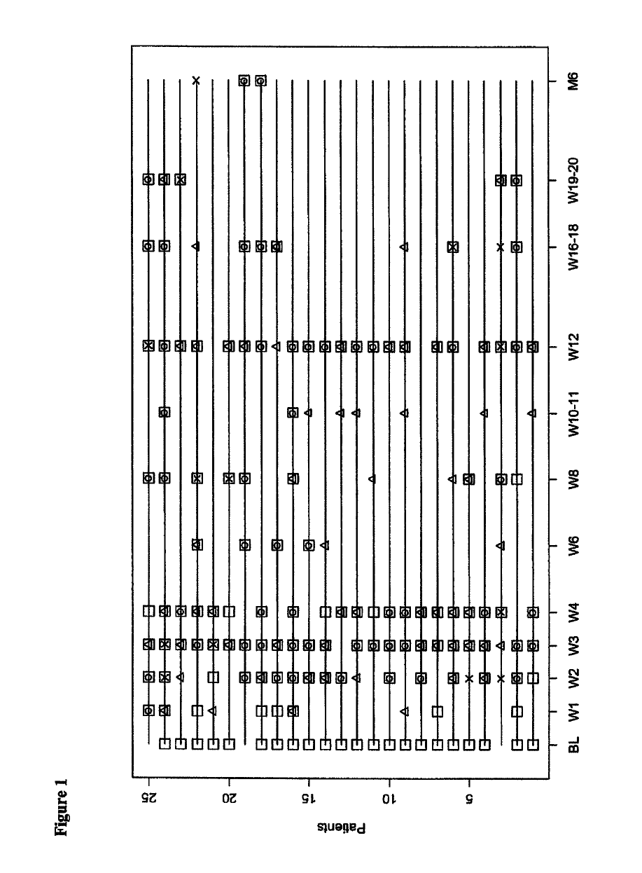

Heart transplant subject data was reviewed and 25 subjects with no serious complications were selected. PAXGene™ blood from time series samples at baseline and weeks 1, 2, 3, 4, 8, and 12 post-transplant was selected for RNA extraction and microarray analysis (FIG. 1).

Subjects and Methods for Proteomic Expression Studies

### Patients

A longitudinal study, approved by the Human Research Ethics Board of the University of British Columbia, was conducted on a series of subjects, with signed consent, who received a cardiac transplant at St. Paul's Hospital, Vancouver, British Columbia between March 2005 and February 2008. Transplant subjects received standard triple immunosuppressive therapy consisting of cyclosporine, prednisone and mycophenolate mofetil. Cyclosporine was replaced by tacrolimus for women and by sirolimus in cases of renal impairment. Basilimax induction was used as a standard protocol. Blood samples were collected prior to transplant and serially for up to 3 years post-transplant, and at times of suspected rejection. Pre-transplant and protocol heart tissue biopsies were collected and placed directly into RNAlater™ Tissue Protect Tubes and stored at −80° C. The biopsies were blindedly reviewed by multiple experienced cardiac pathologists and classified according to the current ISHLT grading scale. Patients with rejection grade ≧2R were identified as having AR for purposes of this investigation. Such patients received appropriate treatments for acute rejection.

The present proteomic study was based on 23 adult cardiac transplant patients with ages ranging from 26 to 70 years, 77% male. Most of these patients were Caucasian (92%); 52% presenting with ischemic heart disease as the primary disease before transplant. Seven patients had at least one acute rejection (AR) with ISHLT Grade ≧2R during the first 5 months post-transplant (AR patients). The other 16 patients did not have an AR episode during same period (NR patients). Samples collected from these 23 patients at different time points resulted in a study cohort of 10 AR samples and 10 NR samples (ISHLT Grade=0R) from AR patients, and 40 NR samples from NR patients.

A potential panel of plasma proteomic markers of cardiac acute rejection was identified using the first timepoint of AR from 6 AR patients and matching timepoints from 12 NR patients. In the internal validation, a test set of samples was constructed using single samples per patient that were randomly selected from the remaining set of samples, resulting in a test set with 11 NR samples from NR patients, and 2 AR samples. Samples available at additional timepoints were used to visualize the properties of the proteomic classifier panel.

### Sample Processing

Blood samples were collected in EDTA tubes, immediately stored on ice. Plasma was obtained within 2 hours from each whole blood sample by centrifugation, aliquoted and stored at −80° C. Peripheral blood plasma drawn from 16 healthy individuals was pooled, aliquoted and stored at −70° C. Heart transplant patient samples were immediately stored on ice before processing and storage at −70° C. within 2 hours. All blood was drawn into tubes with EDTA as an anti-coagulant (BD Biosciences; Franklin Lakes, N.J.). Each plasma sample was then thawed to room temperature, diluted 5 times with 10 mM phosphate buffered saline (PBS) at pH 7.6, and filtered with spin-X centrifuge tube filters (Millipore). Diluted plasma was injected via a 325 μL sample loop onto a 5 mL avian antibody affinity column (Genway Biotech; San Diego, Calif.) to remove the 14 most abundant plasma proteins: albumin, fibrinogen, transferin, IgG, IgA, IgM, haptoglobin, α2-macroglobulin, α1-acid glycoprotein, α1-antitrypsin, Apoliprotein-I, Apoliprotein-II, complement C3 and Apoliprotein B). Flow-through fractions were collected and precipitated by adding TCA to a final concentration of 10% and incubated at 4° C. for 16-18 hours. The protein precipitate was recovered by centrifugation 3200 g at 4° C. for 1 hour, washed three times with ice cold acetone (EMD; Gibbstown, N.J.) and re-hydrated with 200-300 μL iTRAQ buffer consisting of 45:45:10 saturated urea (J. T. Baker; Phillipsburg, N.J.), 0.05 M TEAB buffer (Sigma-Aldrich; St Louis, Mo.), and 0.5% SDS (Sigma-Aldrich; St Louis, Mo.). Each sample was then stored at −70° C. Samples of depleted plasma protein, 100 mg, were digested with sequencing grade modified trypsin (Promega; Madison, Wis.) and labeled with iTRAQ reagents according to manufacturer's protocol (Applied Biosystems; Foster City, Calif.). To ensure interpretable results across different runs, a common reference sample was processed together with 3 patient samples in all runs. The reference sample consisted of a pool of plasma from 16 healthy individuals and was consistently labeled with iTRAQ reagent 114. Patient samples were randomly labeled with iTRAQ reagents 115, 116 and 117. iTRAQ labeled peptides were then pooled and acidified to pH 2.5-3.0. and separated first by strong cation exchange chromatography (PolyLC Inc., Columbia, Md. USA), followed by reverse phase chromatography (Michrom Bioresources Inc., Auburn, Calif. USA) and spotted directly onto 384 spot MALDI ABI 4800 plates, 4 plates per experiment, using a Probot microfraction collector (LC Packings, Amsterdam, Netherlands).

Trypsin Digest and iTRAQ Labeling

Total protein concentration was determined using the bicinchoninic acid assay (BCA) (Sigma-Aldrich, St Louis, Mo. USA) and 100 μg of total protein from each sample was precipitated by the addition of 10 volumes of HPLC grade acetone at −20° C. (Sigma-Aldrich, Seelze, Germany) and incubated for 16-18 hours at −20° C. The protein precipitate was recovered by centrifugation at 16 110×g for 10 min and solubilized in 50 mM TEAB buffer (Sigma-Aldrich; St Louis, Mo.) and 0.2% electrophoresis grade SDS (Fisher Scientific; Fair Lawn, N.J.). Proteins in each sample were reduced with TCEP (Sigma-Aldrich; St Louis, Mo.) at 3.3 mM and incubated at 60° C. for 60 min. Cysteines were blocked with methyl methane thiosulfonate at a final concentration of 6.7 mM and incubated at room temperature for 10 min.

Reduced and blocked samples were digested with sequencing grade modified trypsin (Promega; Madison, Wis.) and incubated at 37° C. for 16-18 hours. Trypsin digested peptide samples were dried in a speed vacuum (Thermo Savant; Holbrook, N.Y.) and labeled with iTRAQ reagent according to the manufacturer's protocol (Applied Biosystems; Foster City, Calif.). Labeled samples were pooled and acidified to pH 2.5-3.0 with concentrated phosphoric acid (ACP Chemicals Inc; Montreal, QC, Canada).

2D-LC Chromatography

iTRAQ labeled peptide were separated by strong cation exchange chromatography (SCX) using a 4.6 mm internal diameter (ID) and 100 mm in length Polysulphoethyl A column packed with 5 μm beads with 300 Å pores (PolyLC Inc., Columbia, Md. USA) on a VISION workstation (Applied Biosystems; Foster City, Calif.). Mobile phases used were Buffer A composed of 10 mM monobasic potassium phosphate (Sigma-Aldrich; St Louis, Mo.) and 25% acetonitrile (EMD Chemicals; Gibbstown, N.J.) pH 2.7, and Buffer B that was the same as A except for the addition of 0.5 M potassium chloride (Sigma-Aldrich St Louis, Mo., USA). Fractions of 500 μL were collected over an 80 minute gradient divided into two linear profiles: 1) 0-30 min with 5% to 35% of Buffer B, and 2) 30-80 min with 35% to 100% of Buffer B. The 20 to 30 fractions with the highest level of peptides, detected by UV trace, were selected and the volume reduced to 150 μL pre fraction. Peptides were desalted by loading fractions onto a C18 PepMap guard column (300 μm ID×5 mm, 5 μm, 100 Å, LC Packings, Amsterdam) and washing for 15 min at 50 μL/min with mobile phase A consisting of water/acetonitrile/TFA 98:2:0.1 (v/v). The trapping column was then switched into the nano flow stream at 200 mL/min where peptides were loaded onto a Magic C18 nano LC column (15 cm, 5 μm pore size, 100 Å, Michrom Bioresources Inc., Auburn Calif., USA) for high resolution chromatography. Peptides were eluted by the following gradient: 0-45 min with 5% to 15% B (acetonitrile/water/TFA 98:2:0.1, v/v); 45-100 min with 15% to 40% B, and 100-105 min with 40% to 75% B. The eluent was spotted directly onto 96 spot MALDI ABI 4800 plates, 4 plates per experiment, using a Probot microfration collector (LC Packings, Amsterdam, Netherlands). Matrix solution, 3 mg/mL α-cyano-4-hydroxycinnamic acid (Sigma-Aldrich, St Louis, Mo. USA) in 50% ACN, 0.1% TFA, was then added at 0.75 μL per spot.

Mass Spectrometry and Data Processing

Peptides spotted onto MALDI plates were analyzed by a 4800 MALDI TOF/TOF analyzer (Applied Biosystems; Foster City, Calif.) controlled using 4000 series Explorer version 3.5 software. The mass spectrometer was set in the positive ion mode with an MS/MS collision energy of 1 keV. A maximum of 1400 shots/spectrum were collected for each MS/MS run causing the total mass time to range from 35 to 40 hours. Peptide identification and quantitation was carried out by ProteinPilot™ Software v2.0 (Applied Biosystems/MDS Sciex, Foster City, Calif. USA) with the integrated new Paragon™ Search Algorithm (Applied Biosystems) (Shilov et al., 2007) and Pro Group™ Algorithm. Database searching was performed against the international protein index (IPI HUMAN v3.39) (Kersey et al, 2004). The precursor tolerance was set to 150 ppm and the iTRAQ fragment tolerance was set to 0.2 Da. Identification parameters were set for trypsin cleavages, cysteine alkylation by MMTS, with special factors set at urea denaturation and an ID focus on biological modifications. The detected protein threshold was set at 85% confidence interval.

Pro Group™ Algorithm (Applied Biosystems) assembled the peptide evidence from the Paragon™ Algorithm into a comprehensive summary of the proteins in the sample and organized the set of identified proteins in protein groups to maintain minimal lists of protein identities within each iTRAQ run. The relative protein levels (protein ratios of concentrations of labels 115, 116 and 117 relative to label 114, respectively) were estimated by Protein Pilot using the corresponding peptide ratios (including singleton peaks). The average protein ratios were calculated by ProteinPilot based on a weighted average of the log ratios of the individual peptides for each protein. The weight of each log ratio was the inverse of the Error Factor, an estimate of the error in the quantitation, calculated by Pro Group Algorithm. This weighted average were then converted back into the linear space and corrected for experimental bias using the Auto Bias correction option in Pro Group Algorithm. Peptide ratios coming from the following cases were excluded from the calculation of the corresponding average protein ratios: shared peptides (i.e., the same peptide sequence was claimed by more than one protein), peptides with a precursor overlap (i.e., the spectrum yielding the identified peptide was also claimed by a different protein but with an unrelated peptide sequence), peptides with a low confidence (i.e., peptide ID confidence <1.0%), peptides that did not have an iTRAQ modification, peptides with only one member of the reagent pair identified, and peptide ratios where the sum of the signal-to-noise ratio for all of the peak pairs was less than 9. Further information on these and other quantitative measures assigned to each protein and on the bias correction are given in ProteinPilot Software documentation.

In this study, plasma proteins, depleted of the 14 most abundant proteins and constituting less than 5% of the total plasma protein mass were analyzed to identify plasma proteomic markers of cardiac acute rejection. As in other shotgun proteomic methods, peptide and protein identification in iTRAQ methodology is based on MS/MS peptide spectra and database searching. Given the ambiguities usually encountered in the protein identification process, many software tools, like ProteinPilot, organize the data by protein groups containing proteins with similar sequences within each experimental run (Nesvizhiskii and Aebersold, 2005). In general, an individual reference name (identifier) is selected as the most likely present protein to represent each group and to be transferred into the protein summary table with corresponding average iTRAQ ratios. However, in some cases, there is no way to differentiate among the different proteins in the group, and in general there is no conclusive evidence about the absence of the non-top proteins in the group. This problem imposes some challenges when matching different replicates as some proteins may appear to be undetected in some replicates when they are truly present but represented by another member of its group. To address this problem and to maximize the number of proteins analyzed a novel algorithm, called Protein Group Code Algorithm (PGCA), was developed. PGCA assigns an identification code to all the proteins in the same protein group within a run and a common code to “similar” protein groups across runs. The assigned protein group code (PGC) was then used to match proteins across different replicates of the experiment. This procedure maintains common identifier nomenclature for related proteins and protein families across all experimental runs.

### Statistical Analysis

A one-protein at a time evaluation of differential relative levels was performed using a robust moderated t-test (eBayes—Smyth GK. Linear models and empirical Bayes methods for assessing differential expression in microarray experiments. Stat Appl Genet Mol Biol. 2004; 3:Article3 (Berkeley Electronic Press) on a set of proteins that, designated by the protein group code assigned by PGCA, had been detected in at least 4 out of 6 AR samples and 8 out of 12 NR samples (i.e., at least ⅔ detection within each analyzed group). eBayes, originally designed for gene expression analysis, decreases the number of false positives caused by artificially low sample variance estimates when the sample size in the study is small. In addition, the robust version of eBayes is less sensitive to observations deviating from the bulk of the data than classical, non-robust tests. Protein group codes with mean relative concentrations (relative to pooled control level) differing significantly between AR and NR (with p-value <0.05) were considered for further analysis. Different criteria were used to identify two potential plasma protein panels: A) false discovery rate (FDR) below 25%, and B) forward selection stepwise discriminant analysis (SDA) maximizing the ability to separate the AR and the NR groups (using R package klaR In R: A language and environment for statistical computing. R Foundation for Statistical Computing, Vienna, Austria).

Linear Discriminant Analysis (LDA) was performed to estimate the ability of the proteomic panels to classify new samples. Two classifiers were built using panels A and B, respectively. The same training (18 subject samples) and test (13 new subject samples) sets were used in both cases (see section Patients). In SDA and LDA, the relative concentration for each protein undetected in patient sample(s) and/or pooled control was imputed using the average relative concentration calculated from training samples in each group (AR and NR means). All of the statistical analyses were implemented using R version 2.6.0.

From the panel of 14 markers, 5 proteins were validated by Enzyme-Linked ImmunoSorbent Assay (ELISA) using commercially available kits and following manufacturer's directions: adiponectin, beta-2 microglobulin, cystatin C (all from R&D Systems, Minneapolis, Minn.), factor X (Diapharma, West Chester, Ohio), and sex hormone-binding globulin (Alpco, Salem, N.H.). Patient samples and the same pooled control used in the iTRAQ experiments were assayed in duplicate by ELISA and analyzed on a VersaMax Tunable Microplate Reader (Molecular Devices, Sunnyvale, Calif.).

### Alloreactive T-Cell Isolation

TABLE 5
Cardiac transplant subject demographics for alloreactive
T-cell gene expression profiling.

| | Subjects with | Subjects without |
|---|---|---|
| | AR (n = 4) | AR (n = 5) |
| Mean Age (standard deviation | 47.38 (15.95) | 59.48 (3.38) |
| Gender (n, % male) | 2 (50%) | 3 (60%) |
| Ethnicity (n, %) | | |
| Caucasian | 4 (100%) | 5 (100%) |
| Primary Disease (n, %) | | |
| Cardiomyopathy - Ischemic | 4 (66.6%) | 3 (60%) |
| (coronary artery disease) | | |
| Cardiomyopathy - Idiopathic | 1 (25%) | — |
| dilated | | |
| Cardiomyopathy - Dilated | — | 1 (20%) |
| Congenital heart disease | — | 1 (20%) |
| Arrhythmogenic (R) Ventricular | 1 (25%) | — |
| Dysplasia | | |

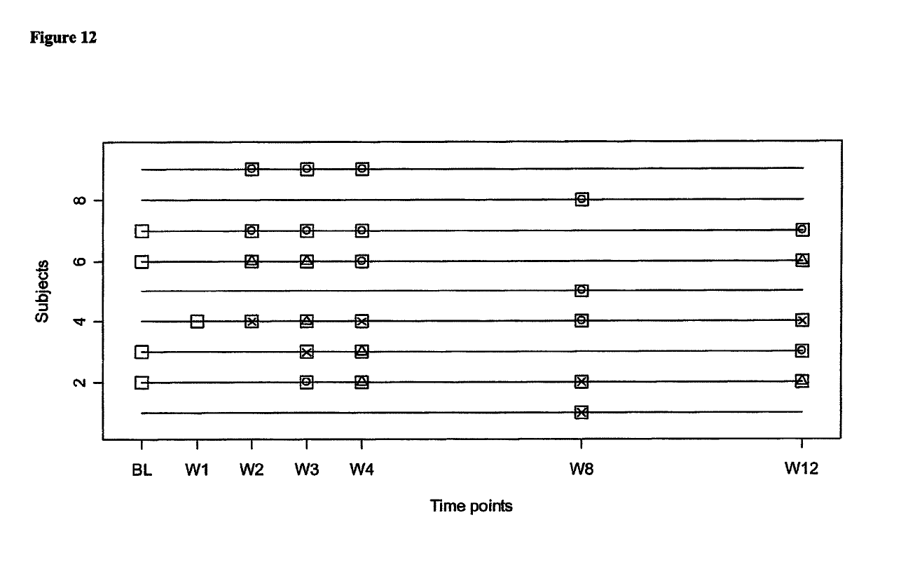

For acute whole blood RNA extraction and microarray analysis, heart transplant subject data was reviewed and 25 subjects with no serious complications were selected. PAXGene™ blood from time series samples at baseline and weeks 1, 2, 3, 4, 8, and 12 post-transplant was selected for RNA extraction and microarray analysis (FIG. 1). For Alloreactive T-cell isolation, RNA extraction and microarray analysis, Blood or spleen samples were collected from consented donors before, at the time, or shortly after transplant. Nine heart transplant subjects were selected for the study based on consent from the donor. This subject distribution and timeline of sampling is illustrated in FIG. 12, subject demographics are indicated in Table 5.

### Biotinylation of APC Membranes

To create biotin coated antigen presenting cell (APC) membranes, white blood cells were first isolated from either donor spleen or donor sodium heparin blood. The cells were then pelleted via centrifugation at 1500 RPM for 5 minutes. A buffer containing 0.2 mg/mL of NHS-biotin (biotin) in PBS was then prepared. The supernatant was removed and the APCs resuspended in biotin solution added at a ratio of 1 μL of buffer per 3000 cells. The tube was inverted a few times for good mixing and incubated at 4° C. for 30 minutes. The tube was then filled with FACS buffer and centrifuged at 1500 RPM for 5 minutes to pellet the cells. The cells were resuspended in FACS buffer and an aliquot removed to determine the extent of biotinylation by staining with SA-PE. The remaining APCs were prepped into membranes as follows. The APC suspension was centrifuged in the 15 mL tubes at 1500 RPM for 5 minutes to pellet the cells. The supernatant was aspirated and the pellet was resuspended in 1 mL of lysis buffer per 2×10⁷ cells. A minimum of 2 mL of lysis buffer was used to make the subsequent homogenization step more efficient. The lysate was allowed to sit on ice for 5 minutes. The cells were then lysed using the Polytron PT 3000 automated homogenizer (Brinkmann). Care was taken to ensure that the generator was fully inserted inside the tube. The RPM were then gradually increased on the homogenizer until a speed is reached at which not much froth is being generated (>10,000 RPM) and the sample was homogenized for 2 minutes at this speed. The contents of the tube were then centrifuged at 2000 RPM for 5 minutes at 4° C. to pellet the remaining non-homogenized cells and unwanted debris. One mL aliquots of supernatant were then transferred into separate 1.5 mL microcentrifuge tubes. These tubes were then centrifuged at 14,000 RPM for 15 minutes at 4° C. to pellet the plasma membranes. The supernatant was aspirated and the pellets were resuspended in 100 μL of a resuspension buffer. Next, a protein determination was performed to quantify the amount of membrane in the solution—an absorbance reading was taken at A280 using a spectrophotometer using 1% BSA as the reference. Resuspension buffer was then used to generate 100 μL aliquots of a cell membrane suspension containing 2 μg of protein per μL.

To ensure adequate biotinylation, an aliquot containing 100,000 cells in 100 μL of FACS buffer was removed and 5 μL of SA-PE added. After mixing by pipetting, the cells were placed on the nutator at 4° C. for 30 minutes in the dark. The tube was then filled with FACS buffer and centrifuged at 1500 RPM for 5 minutes to pellet the cells. The supernatant was then removed and this wash step was repeated twice more to remove any excess SA-PE. Finally, the cells were resuspended in 300 μL of FACS buffer for flow cytometric analysis.

### Binding of Biotinylated APC Membranes to Responder Cells

10 μg of biotinylated membranes were added to each well containing >1.5×10⁵ cells (either PBMCs, PBMCs stained with a fluorochrome conjugated anti-CD3 antibody, or purified CD3+ T cells). The volume of membranes added was usually 5 μL as the membrane preparations were usually stored in aliquots of 200 μg in 100 μL of FACS buffer. The cells were incubated on the nutator for 60 minutes at 4° C. in the dark. The wells were then filled with FACS buffer and the samples centrifuged at 1500 RPM for 5 minutes. The supernatant was removed and more FACS buffer added. This wash step was performed a total of three times. The supernatant was again removed and the cells resuspended in 100 μL of FACS buffer. 2 μL of SA conjugated to a fluorochrome was then added (if the PBMCs were previously stained with a fluorochrome conjugated anti-CD3 antibody, we ensured that the SA conjugated fluorochrome was unique). The samples were incubated on the nutator for 60 minutes at 4° C. in the dark. The wells were then filled with FACS buffer and the samples centrifuged at 1500 RPM for 5 minutes. The supernatant was removed and more FACS buffer added. This wash step was performed a total of three times. The samples were then transferred to the appropriate tube for flow cytometric analysis in 300 μL of FACS buffer.

Extraction of Alloreactive T Cells (Cells that have Bound Biotinylated APC Membranes)

Responder PBMCs that have bound allogeneic biotinylated APC membranes can be isolated using the EasySep® Biotin Selection Kit (StemCell Technologies, Vancouver). This enabled the study of three different subpopulations of responder cells: unmanipulated PBMCs, PBMCs that have bound allogeneic APC membranes (i.e. alloreactive T cells), and PBMCs that have not bound allogeneic APC membranes (i.e. whole PBMCs depleted of alloreactive T cells). In a 15 mL Falcon™ polystyrene round-bottom tube, 1×10⁶ PBMCs were incubated with 300 μg of APC membranes [either from syngeneic (control) or allogeneic (experimental) sources] in 3 mL of staining buffer supplemented with 5 μM of Mg²⁺. on the nutator for 1 hour at 4′C. The tube was then filled with FACS buffer and centrifuged at 1500 RPM for 5 minute and the supernatant was aspirated. This wash step was then repeated again and the cell pellet resuspended in 1 mL of FACS buffer and transferred to a 5 mL Falcon™ polystyrene round-bottom tube. 100 μL of EasySep® Biotin Selection Cocktail (which includes the tetrameric antibody complexes) was then added and the cells incubated at room temperature for 15 minutes. 50 μL of well mixed EasySep® magnetic nanoparticles were then added to the cells and the tube incubated at room temperature for 10 minutes. The tube was then filled to 2.5 mL with FACS buffer and placed inside the EasySep® magnetic for 5 minutes. The tube and magnet were picked up together and the contents of the tube (PBMCs that had not bound biotinylated APC membranes) inverted into a fresh 5 mL tube—this inverted position was held for 3 minutes. This negative fraction contains PBMCs that have not bound the biotinylated APC membranes. The cells bound to the bead comprised the portion of the biological sample enriched for alloreactive T cells, which were then subjected to RNA extraction.

### RNA Extraction and Microarray Analysis

RNA extraction was performed on thawed samples using the PAXgene™ Blood RNA Kit [Cat #762134] to isolate total RNA. Between 4 and 10 μg of RNA was routinely isolated from 2.5 ml whole blood and the RNA quality confirmed using the Agilent BioAnalyzer. Samples with 1.5 μg of RNA, an RIN number >5, and A240/A280 >1.9 were packaged on dry ice and shipped by Federal Express to the Microarray Core (MAC) Laboratory, Children's Hospital, Los Angeles, Calif. for Affymetrix microarray analysis. The microarray analysis was performed by a single technician at the CAP/CLIA accredited MAC laboratory. Nascent RNA was used for double stranded cDNA synthesis. The cDNA was then labeled with biotin, fragmented, mixed with hybridization cocktail and hybridized onto GeneChip Human Genome U133 Plus 2.0 Arrays. The arrays were scanned with the Affymetrix System in batches of 48 with an internal RNA control made from pooled normal whole blood. Microarrays were checked for quality issues using Affymetrix version 1.16.0 and affyPLM version 1.14.0 BioConductor packages (Bolstad, B., Low Level Analysis of High-density Oligonucleotide Array Data: Background, Normalization and Summarization. 2004, University of California, Berkeley; Irizarry et al. 2003. Biostatistics 4(2): 249-64). The arrays with lower quality were repeated with a different RNA aliquot from the same time point. The Affymetrix™ NetAffx™ Annotation database Update Release 25 (March 2008) was used for identification and analysis of microarray results.

### NMR Compound Identification (for Metabolite Studies)

Ultrafiltration of selected serum samples was carried out using a 3 kDa MW 500 μL maximum volume cutoff filter (Pall Life Sciences) in order to separate higher molecular weight components from the metabolites of interest. NMR-ready serum samples were prepared by transferring a 300 μL aliquot of the ultrafiltered fluid to a 1.5 mL Eppendorf tube followed by the addition of 35 μL D₂O and 15 μL of a standard solution (3.73 mM DSS (disodium-2,2-dimethyl-2-silapentane-5-sulphonate), 233 mM imidazole, and 0.47% NaN₃ in H₂O, Sigma-Aldrich, Mississauga, ON). Each serum sample prepared in this manner contained 0.16 mM DSS, 10 mM imidazole, and 0.02% NaN₃ at a pH of 7.3-7.7. The sample (350 μL) was then transferred to a standard SHIGEMI microcell NMR tube for NMR spectra analysis.

All ¹H-NMR spectra were collected on a 500 MHz Inova (Varian Inc., Palo Alto, Calif.) spectrometer equipped with either a 5 mm HCN Z-gradient pulsed-field gradient (PFG) room-temperature probe or a Z-gradient PFG Varian cold-probe. ¹H-NMR spectra were acquired at 25° C. using the first transient of the tnnoesy-presaturation pulse sequence, which was chosen for its high degree of high quantitative accuracy (E. J. Saude, C. M. Slupsky, B. D. Sykes, Metabolomics 2 (2006) 113.). Spectra were collected with 64 transients using a 4 s acquisition time and a 1 s recycle delay. For certain confirmatory experiments, higher field (800 MHz Varian Inova) instruments and larger numbers of transients (256) were used.

Prior to spectral analysis, all FIDs were zero-filled to 64 k data points, and a line broadening of 0.5 Hz was applied. The methyl singlet of the buffer constituent DSS served as an internal standard for chemical shift referencing (set to 0 ppm) and for quantification. All ¹H-NMR spectra were processed and analyzed using the Chenomx NMR Suite Professional software package version 4.6 (Chenomx Inc., Edmonton, AB). The Chenomx NMR Suite software allows for qualitative and quantitative analysis of an NMR spectrum by “fitting” spectral signatures from an internal database of reference spectra to the full NMR spectrum (A. M. Weljie, J. Newton, P. Mercier, E. Carlson, C. M. Slupsky, Anal. Chem. 78 (2006) 4430). Spectral fitting for each metabolite was done using the standard Chenomx 500 MHz (pH 6-8) metabolite library. Concentration data was corrected for bandpass filter attenuation as previously described (E. J. Saude, B. D. Sykes, Metabolomics 3 (2007) 19). In addition to these checks, sample spiking was used to confirm the identity of many spectral signatures seen in the NMR spectra. Sample spiking was performed by adding 20-200 μM of the presumptive compound to selected serum samples and checking to see if the corresponding ¹H NMR signals changed as expected.

### Statistical Analysis

The statistical analysis was performed using SAS version 9.1, R version 2.6.1 and BioConductor version 2.1 (Gentleman, R., et al., Genome Biology, 2004. 5: p. R80).

For analysis of genomic and T-Cell microarray data, Robust Multi-array Average (RMA) (Bolstad, et al. Bioinformatics, 2003. 19(2): p. 185-93) technique was used for background correction, normalization and summarization as available in the Affymetrix BioConductor package. A noise minimization was then performed; probe sets with expression values consistently lower than 50 across at least 3 samples were considered as noise and eliminated from further analysis. The remaining probe sets were analyzed using three different moderated T-tests. Two of the methods are available in the Linear Models for Microarray data (limma) BioConductor package—robust fit combined with eBayes and least square fit combined with eBayes. The third statistical analysis method, Statistical Analysis of Microarrays (SAM), is available in the same BioConductor package. A gene was considered statistically significant if it had a false discovery rate (FDR)<0.05 in all three methods and a fold change >2 in all three moderated T-tests (Smyth, G., Limma: linear models for microarray data, in Bioinformatics and Computational Biology Solutions using R and Bioconductor, R. Gentleman, et al., Editors. 2005, Springer: New York). The biomarker panel genes were identified by applying Stepwise Discriminant Analysis (SDA) with forward selection on the statistically significant probe sets. Linear Discriminant Analysis (LDA) was used to train and test the biomarker panel as a classifier.

The metabolite data was analyzed in two different ways. First, the absolute concentration of the acute rejection (AR) sample (ISHLT grading ≧2R) was compared to the non-rejection (NR) samples (ISHLT grade 0R). Second, the relative to baseline concentration of AR samples was compared to the relative to baseline concentration of NR samples. The relative concentration is calculated for each subject by dividing the post-transplant sample's concentration value by the baseline sample's concentration level. For each analysis two different moderated T-test was used and in both analyses, metabolites with an FDR (false discovery rate)<0.05 were considered statistically significant. The two different t-tests were Significance Analysis of Microarrays (SAM) and robust eBayes. Metabolites were deemed to be significant from either t-test. SAM identified the metabolites significant for the relative to baseline concentration data, and robust eBayes t-test identified the metabolites significant for the absolute concentration data.

### Example 1

### Genomic Expression Profiling

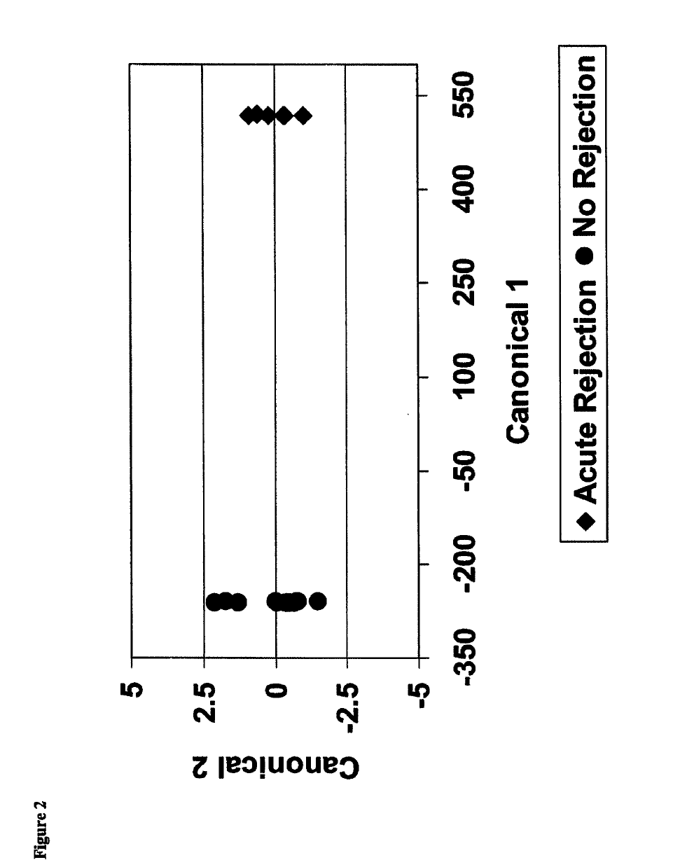

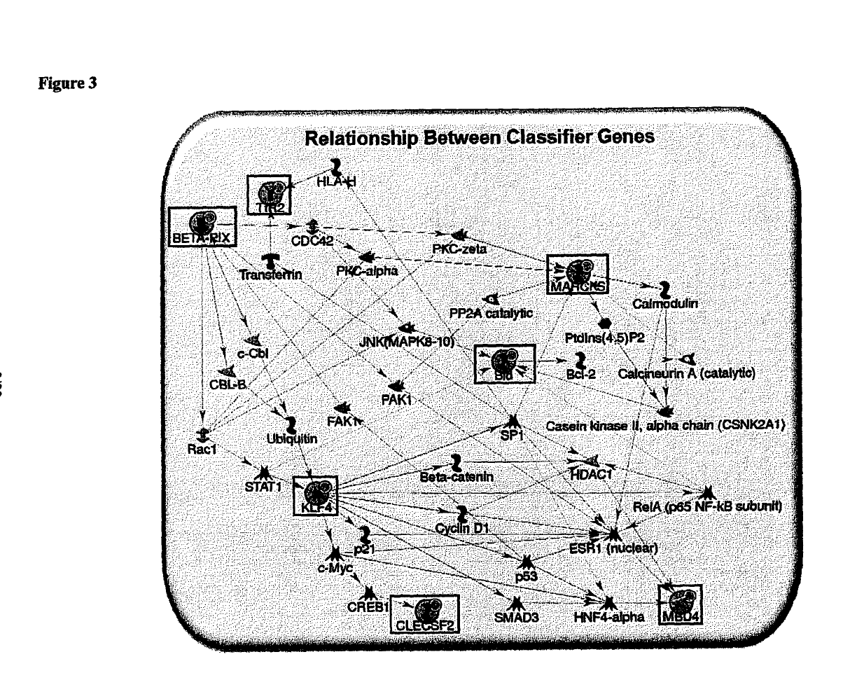

39 differentially expressed probe sets were identified, each of which demonstrated at a least 2-fold difference between samples from acute rejection patients (AR) and those from non-rejection patients (NR) (Table 6). A subset of twelve markers was identified which consistently differentiated AR and NR subjects (indicated in Table 6 with “++”). As per FIG. 2, the increase or decrease in the TRF2, SRGAP2P1, KLF4, YLPM1, BID, MARCKS, CLEC2B, ARHGEF7, LYPLAL1, WRB, FGFR1OP2 and MBD4 markers allowed for categorization of each sample as an AR or NR.

TABLE 6
Differentially expressed probe sets, exhibiting at least a 2-fold difference between AR and NR subjects.
The target sequence is the portion of the consensus or exemplar sequence from which the probe
sequences were selected. The consensus or exemplar Sequence is the sequence used at the time of
design of the array to represent the transcript that the GeneChip U133 2.0 probe set measures. A
consensus sequence results from base-calling algorithms that align and combine sequence data into
groups. An exemplar sequence is a representative cDNA sequence for each gene.

| | | | | | | Direction | SEQ ID NO: |
|---|---|---|---|---|---|---|---|
| Affymetrix | | | RefSeq Accession | log2 (Fold | Fold | (AR versus | of Target |
| Probe Set ID | Gene Symbol | Gene Title | No. | Change) | Change | NR) | sequence |
| 207883_s_at | ++TFR2 | transferrin receptor 2 | NM_003227 | 1.05 | 2.07 | up | 25 |
| 229067_at | ++SRGAP2P1 | SLIT-ROBO Rho | XM_209227 | −1.73 | 3.33 | down | 26 |
| | | GTPase activating | | | | | |
| | | protein 2 | | | | | |
| | | pseudogene 1 | | | | | |
| 221841_s_at | ++KLF4 | Kruppel-like factor 4 | NM_004235 | −1.46 | 2.75 | down | 27 |
| | | (gut) | | | | | |
| 214659_x_at | ++YLPM1 | YLP motif | NM_019589 | −1.02 | 2.03 | down | 28 |
| | | containing 1 | XM_930487 | | | | |
| | | | XM_940570 | | | | |
| 204493_at | ++BID | BH3 interacting | NM_001196 | −1.01 | 2.01 | down | 29 |
| | | domain death | NM_197966 | | | | |
| | | agonist | NM_197967 | | | | |
| 201669_s_at | ++MARCKS | myristoylated | NM_002356 | −1.51 | 2.84 | down | 30 |
| | | alanine-rich protein | | | | | |
| | | kinase C substrate | | | | | |
| 1556209_at | ++CLEC2B | “C-type lectin | NM_005127 | −1.20 | 2.29 | down | 31 |
| | | domain family 2, | | | | | |
| | | member B” | | | | | |
| 235412_at | ++ARHGEF7 | Rho guanine | NM_003899 | −1.12 | 2.17 | down | 32 |
| | | nucleotide exchange | NM_145735 | | | | |
| | | factor (GEF) 7 | | | | | |
| 226851_at | ++LYPLAL1 | lysophospholipase- | NM_138794 | −1.12 | 2.17 | down | 33 |
| | | like 1 | | | | | |
| 202749_at | ++WRB | tryptophan rich basic | NM_004627 | −1.07 | 2.09 | down | 34 |
| | | protein | | | | | |
| 1556283_s_at | ++FGFR1OP2 | FGFR1 oncogene | NM_015633 | 1.36 | 2.56 | up | 35 |
| | | partner 2 | | | | | |
| 209580_s_at | ++MBD4 | methyl-CpG binding | NM_003925 | −1.01 | 2.02 | down | 36 |
| | | domain protein 4 | | | | | |
| 205884_at | ITGA4 | “integrin, alpha 4 | NM_000885 | −1.21 | 2.31 | down | 308 |
| | | (antigen CD49D, | | | | | |
| | | alpha 4 subunit of | | | | | |
| | | VLA-4 receptor)” | | | | | |
| 1553530_a_at | ITGB1 | “integrin, beta 1 | NM_002211 | −1.22 | 2.32 | down | 309 |
| | | (fibronectin receptor, | NM_033666 | | | | |
| | | beta polypeptide, | NM_033667 | | | | |
| | | antigen CD29 | NM_033668 | | | | |
| | | includes MDF2, | NM_033669 | | | | |
| | | MSK12)” | NM_133376 | | | | |
| 203741_s_at | ADCY7 | adenylate cyclase 7 | NM_001114 | −1.32 | 2.49 | down | 310 |
| 200604_s_at | PRKAR1A | “protein kinase, | NM_002734 | −1.16 | 2.23 | down | 311 |
| | | cAMP-dependent, | NM_212471 | | | | |
| | | regulatory, type I, | NM_212472 | | | | |
| | | alpha (tissue specific | | | | | |
| | | extinguisher 1)” | | | | | |
| 202742_s_at | PRKACB | “protein kinase, | NM_002731 | −1.11 | 2.16 | down | 312 |
| | | cAMP-dependent, | NM_182948 | | | | |
| | | catalytic, beta” | NM_207578 | | | | |
| 1555814_a_at | RHOA | “ras homolog gene | NM_001664 | −1.27 | 2.41 | down | 313 |
| | | family, member A” | | | | | |
| 1567458_s_at | RAC1 | “ras-related C3 | NM_006908 | −1.15 | 2.21 | down | 314 |
| | | botulinum toxin | NM_018890 | | | | |
| | | substrate 1 (rho | NM_198829 | | | | |
| | | family, small GTP | | | | | |
| | | binding protein | | | | | |
| | | Rac1)” | | | | | |
| 201604_s_at | PPP1R12A | “protein phosphatase | NM_002480 | −1.33 | 2.51 | down | 315 |
| | | 1, regulatory | | | | | |
| | | (inhibitor) subunit | | | | | |
| | | 12A” | | | | | |
| 211985_s_at | CALM1 | “calmodulin 1 | NM_006888 | −1.18 | 2.27 | down | 316 |
| | | (phosphorylase | | | | | |
| | | kinase, delta)” | | | | | |
| 210088_x_at | MYL4 | “myosin, light chain | NM_001002841 | 1.04 | 2.06 | up | 317 |
| | | 4, alkali; atrial, | NM_002476 | | | | |
| | | embryonic” | | | | | |
| 210395_x_at | MYL4 | “myosin, light chain | NM_001002841 | 1.02 | 2.02 | up | 318 |
| | | 4, alkali; atrial, | NM_002476 | | | | |
| | | embryonic” | | | | | |
| 216054_x_at | MYL4 | “myosin, light chain | NM_001002841 | 1.12 | 2.17 | up | 319 |
| | | 4, alkali; atrial, | NM_002476 | | | | |
| | | embryonic” | | | | | |
| 217274_x_at | MYL4 | “myosin, light chain | NM_001002841 | 1.05 | 2.07 | up | 320 |
| | | 4, alkali; atrial, | NM_002476 | | | | |
| | | embryonic” | | | | | |
| 215719_x_at | FAS | “Fas (TNF receptor | NM_000043 | −1.59 | 3.00 | down | 321 |
| | | superfamily, member | NM_152871 | | | | |
| | | 6)” | NM_152872 | | | | |
| | | | NM_152873 | | | | |
| | | | NM_152874 | | | | |
| | | | NM_152875 | | | | |
| | | | NM_152876 | | | | |
| | | | NM_152877 | | | | |
| 216252_x_at | FAS | “Fas (TNF receptor | NM_000043 | −1.26 | 2.40 | down | 322 |
| | | superfamily, member | NM_152871 | | | | |
| | | 6)” | NM_152872 | | | | |
| | | | NM_152873 | | | | |
| | | | NM_152874 | | | | |
| | | | NM_152875 | | | | |
| | | | NM_152876 | | | | |
| | | | NM_152877 | | | | |
| 222201_s_at | CASP8AP2 | CASP8 associated | NM_012115 | −1.49 | 2.81 | down | 323 |
| | | protein 2 | | | | | |
| 207686_s_at | CASP8 | “caspase 8, | NM_001080124 | −1.28 | 2.42 | down | 324 |
| | | apoptosis-related | NM_001080125 | | | | |
| | | cysteine peptidase” | NM_001228 | | | | |
| | | | NM_033355 | | | | |
| | | | NM_033356 | | | | |
| | | | NM_033358 | | | | |
| 222547_at | MAP4K4 | mitogen-activated | NM_004834 | −1.03 | 2.04 | down | 325 |
| | | protein kinase kinase | NM_145686 | | | | |
| | | kinase kinase 4 | NM_145687 | | | | |
| 214786_at | MAP3K1 | mitogen-activated | NM_005921 | −1.11 | 2.16 | down | 326 |
| | | protein kinase kinase | XM_001128827 | | | | |
| | | kinase 1 | XM_042066 | | | | |
| 227761_at | MYO5A | “myosin VA (heavy | NM_000259 | −1.58 | 3.00 | down | 327 |
| | | chain 12, myoxin)” | | | | | |
| 203218_at | MAPK9 | mitogen-activated | NM_002752 | −1.14 | 2.21 | down | 328 |
| | | protein kinase 9 | NM_139068 | | | | |
| | | | NM_139069 | | | | |
| | | | NM_139070 | | | | |
| 1552610_a_at | JAK1 | Janus kinase 1 (a | NM_002227 | −1.21 | 2.31 | down | 329 |
| | | protein tyrosine | | | | | |
| | | kinase) | | | | | |
| 200796_s_at | MCL1 | myeloid cell | NM_021960 | −1.46 | 2.76 | down | 330 |
| | | leukemia sequence 1 | NM_182763 | | | | |
| | | (BCL2-related) | | | | | |
| 200798_x_at | MCL1 | myeloid cell | NM_021960 | −1.61 | 3.04 | down | 331 |
| | | leukemia sequence 1 | NM_182763 | | | | |
| | | (BCL2-related) | | | | | |
| 235982_at | FCRL1 | Fc receptor-like 1 | NM_052938 | −1.12 | 2.17 | down | 332 |
| 201551_s_at | LAMP1 | lysosomal-associated | NM_005561 | −1.32 | 2.49 | down | 333 |
| | | membrane protein 1 | | | | | |
| 220342_x_at | EDEM3 | “ER degradation | NM_025191 | −1.05 | 2.07 | down | 334 |
| | | enhancer, | | | | | |
| | | mannosidase alpha- | | | | | |
| | | like 3” | | | | | |
| 217234_s_at | VIL2 | villin 2 (ezrin) | NM_001111077 | −1.16 | 2.23 | down | 335 |
| | | | NM_003379 | | | | |
| 235167_at | DKEZp547E087 | hypothetical gene | XM_496136 | −1.31 | 2.48 | down | 336 |
| | | LOC283846 | XM_931802 | | | | |
| | | | XM_931808 | | | | |
| | | | XM_931814 | | | | |
| | | | XM_931818 | | | | |
| | | | XM_931827 | | | | |
| | | | XM_931837 | | | | |
| | | | XM_931840 | | | | |
| 214697_s_at | ROD1 | ROD1 regulator of | NM_005156 | −1.82 | 3.54 | down | 337 |
| | | differentiation 1 (S. pombe) | | | | | |
| 221561_at | SOAT1 | sterol O- | NM_003101 | −1.82 | 3.52 | down | 338 |
| | | acyltransferase (acyl- | | | | | |
| | | Coenzyme A: | | | | | |
| | | cholesterol | | | | | |
| | | acyltransferase) 1 | | | | | |
| 201222_s_at | RAD23B | RAD23 homolog B | NM_002874 | −1.36 | 2.57 | down | 339 |
| | | (S. cerevisiae) | | | | | |
| 204156_at | KIAA0999 | KIAA0999 protein | NM_025164 | −1.11 | 2.16 | down | 340 |
| 1553685_s_at | SP1 | Sp1 transcription | NM_138473 | −1.43 | 2.70 | down | 341 |
| | | factor | | | | | |
| 1554834_a_at | RASSF5 | Ras association | NM_031437 | −1.41 | 2.67 | down | 342 |
| | | (RalGDS/AF-6) | NM_182663 | | | | |
| | | domain family 5 | NM_182664 | | | | |
| | | | NM_182665 | | | | |
| 1557910_at | HSP90AB1 | “heat shock protein | NM_007355 | −1.31 | 2.47 | down | 343 |
| | | 90 kDa alpha | | | | | |
| | | (cytosolic), class B | | | | | |
| | | member 1” | | | | | |
| 222150_s_at | tcag7.1314 | hypothetical protein | NM_017439 | −1.85 | 3.62 | down | 344 |
| | | LOC54103 | | | | | |

### Example 2

### Biological Pathways Based on Genomic Expression Profiling

Using a combination of bioinformatics and literature-based approaches, various pathways have been identified based on selected differentially expressed genes. Interactions between them have also been elucidated in our current results. FIG. 3 illustrates a pathway-based relationship between the biomarkers ARHGEF7, TRF2, BID, MARCKS, KLF4, CLEC2B and MBD4.

Interactions between the biomarker genes and/or gene products:

### 1. BETAPIX→Rac1→STAT1→KLF4

BETA-PIX→Rac 1 (Park et al, 2004. Mol Cell Biol 24:4384-94)
Rac1→STAT1→KLF4 (Uddin et al, 2000 J. Biol Chem 275:27634-40; Feinberg et al 2005. J. Biol. Chem. 280:38247-58)
   2. KLF4→(c-MYC→CREB1)→CLECSF2
KLF4→c-MYC (Kharas et al 2007. Blood. 109:747-55)
c-MYC→CREB1 (Tamura et al 2005 EMBO J. 24:2590-601)
CREB1→CLECSF2 (Zhang et al 2005. Proc Natl Acad. Sci. 102:4459-64)

### 3. STAT1→BID

STAT1→KLF4 (Uddin et al, 2000 J. Biol Chem 275:27634-40; Feinberg et al 2005. J. Biol. Chem. 280:38247-58)
STAT1→BID (Hartmann et al 2005. Genes & Development 19:2953-2968)

### 4. KLF→Beta-catenin→HDAC1→MBD4

KLF→beta catenin (Zhang et al, 2006. Mol. Cell Biol. 26:2055-64)
beta-catenin→HDAC1 (Baek et al 2003. Proc Natl Acad Sci 100:3245-50)
HDAC1 MBD4 (Kondo et al 2005. mo. Cell Biol 25:4388-96)

### 5. BETA-PIX→CDC42→PKC-zeta→MARCKS

BETA-PIX→CDC42 (Feng et al 2002. J Biol Chem 277:5644-50)
CDC42→PKC-zeta (Slater et al 2001. Biochemistry 40:4437-45)
PKC-zeta→MARCKS (Hartwig et al 1992. Nature 356:618-22)

### 6. KLF4→SP1→HLA-H→TfR2

KLF4→SP1 (Kanai et al 2006. Clin Cancer Res 12:6395-402)
SP1→HLA-H (Mura et al 2004. FASEB J. 18:1922-4)
HLA-H→TFR2 (Goswami et al 2006. J. Biol. Chem. 281:28494-8)

### Example 3

### Metabolite Profiling

Metabolite profiles of subjects were obtained as described. 33 metabolites (Table 3) were identified and quantified in 53 serum samples obtained from the subject population. Comparisons between AR and NR subject samples. Subject samples were identified as AR or NR based on ISHLT biopsy score (≧2R for AR, 0R for NR). ISHLT biopsy scores are determined by a pathologist's assessment of an endomyocardial biopsy (Stewart et al 2005, supra.)

Metabolites exhibiting a statistically significant change are listed in Tables 7a-d.

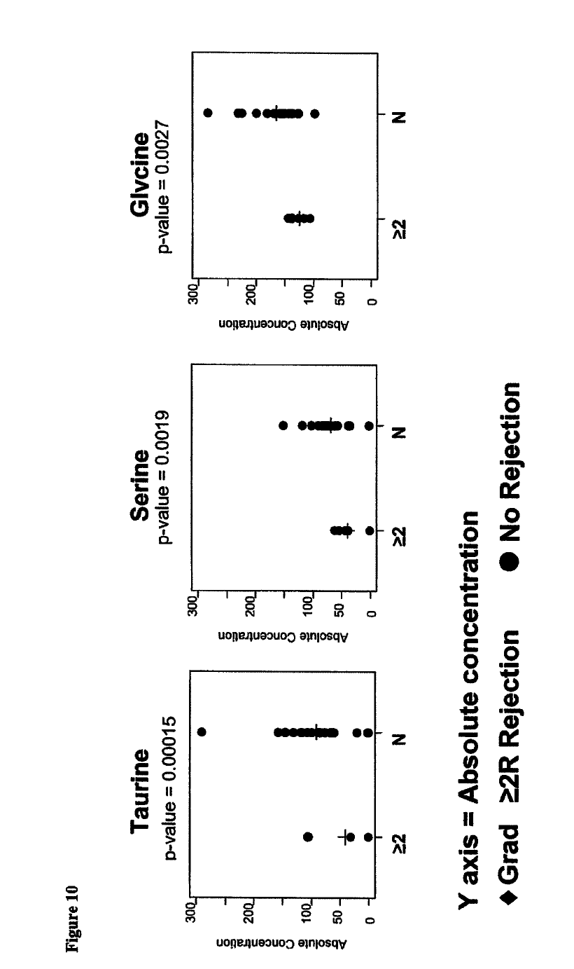

As illustrated in FIG. 10, the absolute concentration for each of taurine, serine and glycine allowed for determination of the rejection status of each of the subjects in the population tested. All subjects having an ISHLT biopsy score ≧2R were correctly assigned a rejection status of AR; while all subjects having an ISHLT biopsy score 0R were correctly assigned a rejection status of NR by metabolite profiling.

When the concentration of the post-transplant sample was compared to the baseline concentration, three metabolites were statistically significant using a moderated t-test. The line illustrates the mean of each group. The total sample population included six samples from AR subjects and 21 from NR subjects.

TABLE 7a
Absolute concentration values for taurine,
serine and glycine in AR and NR subjects.
Absolute concentration (micromolar)

| Metabolite | Taurine | Serine | Glycine |
|---|---|---|---|
| AR1 | 6.727920455 | 5.321928095 | 7.118941073 |
| AR2 | −4.321928095 | 5.426264755 | 6.87036472 |
| AR3 | 6.714245518 | 5.754887502 | 6.727920455 |
| AR4 | −4.321928095 | −4.321928095 | 6.87036472 |
| AR5 | 4.95419631 | 5.321928095 | 6.988684687 |
| AR6 | −4.321928095 | 5.95419631 | 7.17990909 |
| NR1 | −4.321928095 | 5.169925001 | 7.247927513 |
| NR2 | 7.17990909 | 6.321928095 | 7.169925001 |
| NR3 | 7.17990909 | 6.321928095 | 7.499845887 |
| NR4 | −4.321928095 | 6.108524457 | 7.108524457 |
| NR5 | 6.06608919 | 6.189824559 | 7.321928095 |
| NR6 | 7.199672345 | 6.894817763 | 7.864186145 |
| NR7 | 6.475733431 | 6.672425342 | 7.392317423 |
| NR8 | 6.459431619 | 7.247927513 | 8.154818109 |
| NR9 | 7.294620749 | 6.375039431 | 7.813781191 |
| NR10 | 6.727920455 | 6.189824559 | 7.64385619 |
| NR11 | 6.392317423 | −4.321928095 | 6.988684687 |
| NR12 | 6.614709844 | 5.906890596 | 7.169925001 |
| NR13 | 6.87036472 | −4.321928095 | 7.169925001 |
| NR14 | 8.184875343 | 6.169925001 | 7.169925001 |
| NR15 | 4.321928095 | −4.321928095 | 6.62935662 |
| NR16 | 7.022367813 | 6.375039431 | 7.276124405 |
| NR17 | 5.882643049 | 5.781359714 | 7 |
| NR18 | −4.321928095 | 6.209453366 | 7.409390936 |
| NR19 | 6.247927513 | 6.044394119 | 7.098032083 |
| NR20 | 5.977279923 | 5.209453366 | 7.247927513 |
| NR21 | 6.857980995 | 6.189824559 | 7.294620749 |
| NR22 | −4.321928095 | 6.475733431 | 7.499845887 |

TABLE 7b
Heart metabolite markers - Absolute Concentration: mean,
std dev for AR and NR subject data of Table 7a.

| Metabolite | mean(AR) | SD(AR) | mean(NR) | SD(NR) |
|---|---|---|---|---|
| Taurine | 0.905 | 5.762 | 4.621 | 4.375 |
| Serine | 3.909 | 4.040 | 4.767 | 3.724 |
| Glycine | 6.959 | 0.169 | 7.325 | 0.331 |

TABLE 7c
Relative to baseline concentration values for glycine,
creatine and carnitine in AR and NR subjects
Relative concentration

| Metabolite | Glycine | Creatine | Carnitine |
|---|---|---|---|
| AR1 | 0.391020618 | 0.584962501 | 0.494764692 |
| AR2 | −0.01227833 | 2.887525271 | 1.600392541 |
| AR3 | −0.154722595 | 2.263034406 | 1.293731203 |
| AR4 | −0.01227833 | 0.093109404 | −1.632268215 |
| AR5 | −2.613086102 | −0.900464326 | 1.070389328 |
| AR6 | −2.421861698 | −0.637429921 | 1.263034406 |
| NR1 | 0.520007059 | −0.415037499 | 0.125530882 |
| NR2 | 0.442004547 | 1 | 0.750021747 |
| NR3 | 0.617202838 | 0 | −0.494764692 |
| NR4 | −2.493246332 | −0.559427409 | 1.070389328 |
| NR5 | −2.279842694 | −1.807354922 | 0.765534746 |
| NR6 | 0.588061739 | −1.125530882 | 0.649502753 |
| NR7 | 0.116193018 | −0.702319451 | 0.349942471 |
| NR8 | 0.878693704 | −0.803602787 | −0.718229032 |
| NR9 | 0.537656786 | −2.263034406 | −0.628031223 |
| NR10 | 0.192645078 | −1.280107919 | −1.371968777 |
| NR11 | −0.181240315 | −1.137503524 | 0.061400545 |
| NR12 | 0.455679484 | 0 | −0.134301092 |
| NR13 | 0.455679484 | 0.099535674 | 0.263034406 |
| NR14 | 0.455679484 | 1.618909833 | −0.032421478 |
| NR15 | −0.084888898 | 0.099535674 | −1.584962501 |
| NR16 | 0.116253068 | −0.308122295 | −0.359081093 |
| NR17 | −0.159871337 | −2.115477217 | −0.928446739 |
| NR18 | 0.249519599 | −0.176877762 | −1.560714954 |
| NR19 | −0.031250934 | 1.365649472 | 0.584962501 |
| NR20 | 0.118644496 | −0.378511623 | −0.308122295 |
| NR21 | 0.165337732 | −0.378511623 | −0.378511623 |
| NR22 | 0.37056287 | −0.893084796 | 0.791413378 |

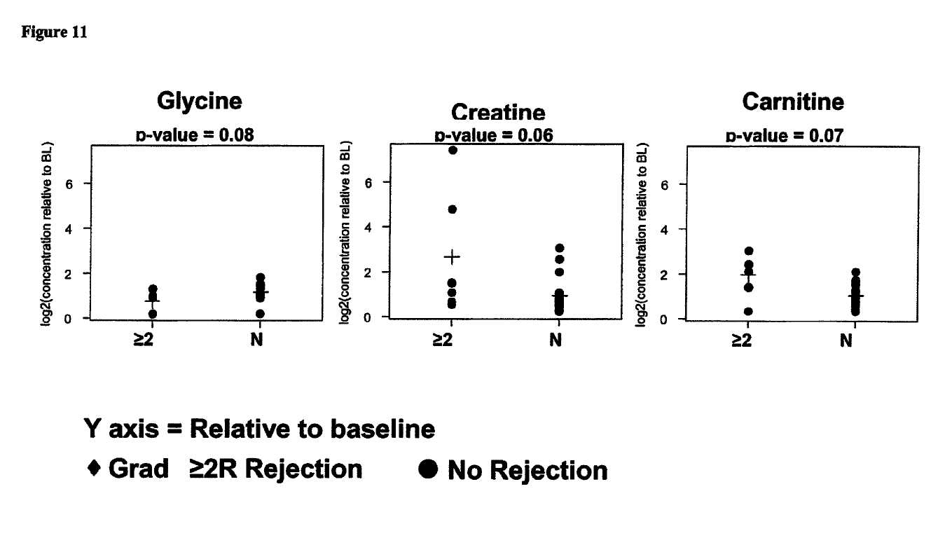

As illustrated in FIG. 11, the relative to baseline concentration for each of glycine, creatine and carnitine allowed for determination of the rejection status of each of the subjects in the population tested. All subjects having an ISHLT biopsy score ≧2R were correctly assigned a rejection status of AR; while all subjects having an ISHLT biopsy score 0R were correctly assigned a rejection status of NR.

TABLE 7d
Heart metabolite markers - Relative to baseline Concentration:
mean, std dev for AR and NR subject data of Table 7c.

| Metabolite | mean(AR) | SD(AR) | mean(NR) | SD(NR) |
|---|---|---|---|---|
| Glycine | −0.803 | 1.341 | 0.0477 | 0.834 |
| Creatine | 0.715 | 1.546 | −0.461 | 0.993 |
| Carnitine | 0.681 | 1.191 | −0.140 | 0.777 |

TABLE 8
Magnitude and direction of fold-change

| | | Fold Change | Direction |
|---|---|---|---|
| AR and NR comparision method | Metabolite | (AR versus NR) | (AR versus NR) |
| Absolute concentration based analysis | Taurine | 0.444 | Down |
| Absolute concentration based analysis | Serine | 0.593 | down |
| Absolute concentration based analysis | Glycine | 0.759 | down |
| Relative to baseline concentration based analysis | Glycine | 0.657 | down |
| Relative to baseline concentration based analysis | Creatine | 2.890 | up |
| Relative to baseline concentration based analysis | Carnitine | 1.893 | up |

“Absolute concentration” is a comparison between AR and NR samples. “Relative to baseline concentration is a ratio of AR/BL or NR/BL, followed by a comparison of the resulting ratios. When assessed using the absolute concentration method, creatine and carnitine do not exhibit a significant change (data not shown). When metabolites are assessed using the relative to baseline method, taurine and serine do not exhibit a significant change (data not shown).

Higher level of creatine was found in ARs as compared to NR (Table 8)—this may be a reflection of the creatine kinase (CK) level in the AR patients. Upregulation of CK has been used clinically to indicate injury to the skeletal or heart muscle (i.e. in myocardial infarction). Since acute rejection would involve immune-mediated insults to the transplanted organ, it is possible that like CK, creatine is also increased in ARs (relative to NRs) as another indication of allograft damage.

Taurine levels were found to be lower in AR subjects (relative to NRs) (Table 8). Given that low level of taurine has been found in condition such as hypertension, it may be possible that taurine can serve, rather as a specific indicator of increased pressure in the heart, a general biomarker for heart under stress.

It may be possible that the increased level of carnitine seen in rejection patients is partly due to the (compensatory) response of the allograft—to upregulate the fat utilization and thus generating more energy for the heart to counteract the negative effects ischemia/reperfusion, oxygen radical generation and alloimmune response can have on the myocardial energy metabolism.

The above results provide further evidence that differentially expressed level of taurine may serve as a biomarker of allograft rejection (especially considering higher levels of taurine were observed in NRs in our data). Based on the aforementioned study by Rashke et al., it is biologically plausible that the NRs benefited from increased level of taurine which ultimately protects the heart from PMN-induced reperfusion injury and oxidative stress.

Without wishing to be bound by theory, the above results may suggest that, given the role of glycine in production of biopolymers, a subject may exhibit additional demand for glycine to support or upregulate the production of DNA and phospholipids (e.g. for cell membranes) to meet the requirements of the immune cells (e.g. CD4+ and 8+ cells, NK cells and the like) involved in an allograft rejection response. Alternatively, glycine level is lower in AR than NR, possibly because the allograft rejection response and damage to the allograft have disrupted the normal cellular metabolism and energy production of the surrounding recipient cells and tissues.

### Example 4

### Alloreactive T-Cell Profiling

200 probe sets corresponding to 196 genes were differentially expressed between alloreactive T cell samples belonging to AR and NR samples (p>0.01). Based on the expression values of these probe sets, the AR subject samples clustered together separately from the NR subject samples (data not shown). 239901_at 241732_at and 237060_at may represent previously unidentified transcripts or genes specific to alloreactive T cells, or otherwise present in sufficiently low copy number so as to be masked using conventional techniques.

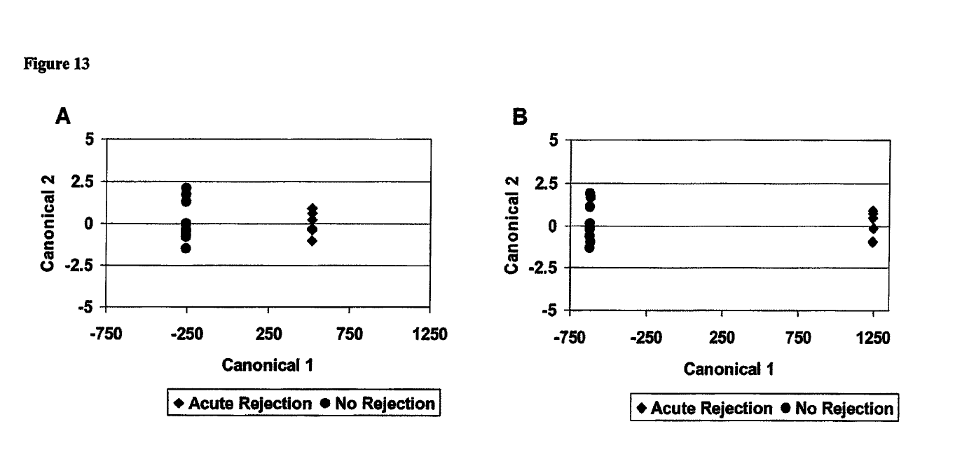

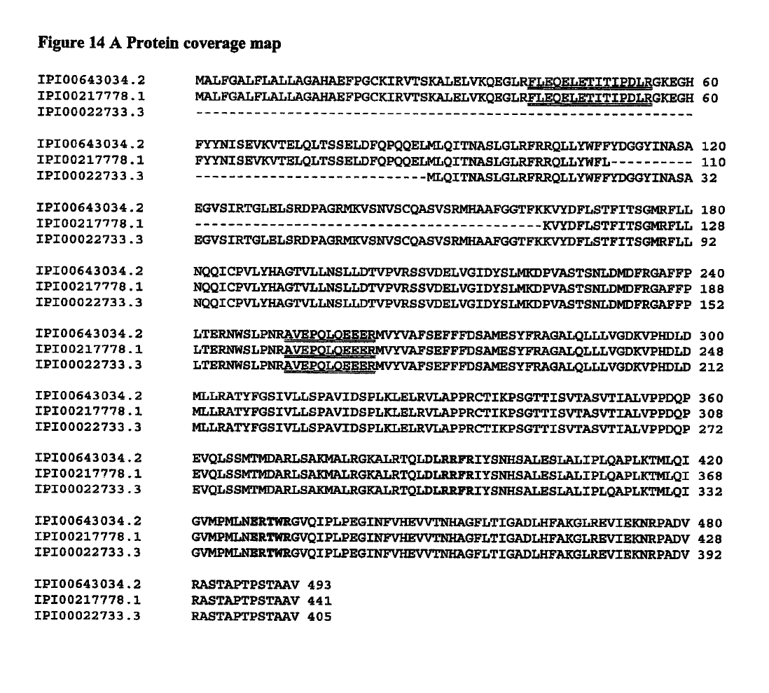

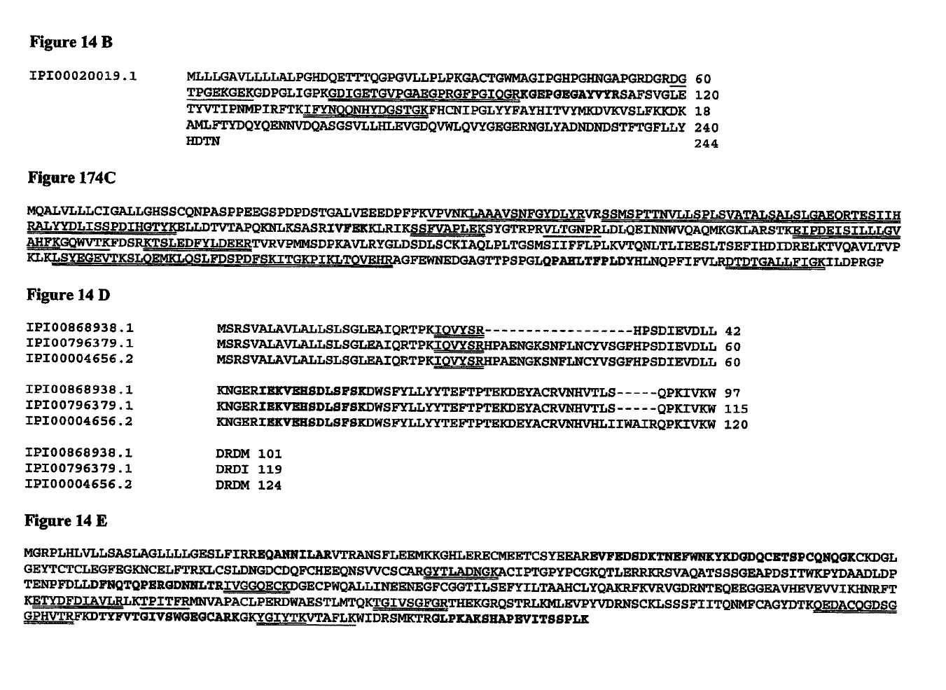

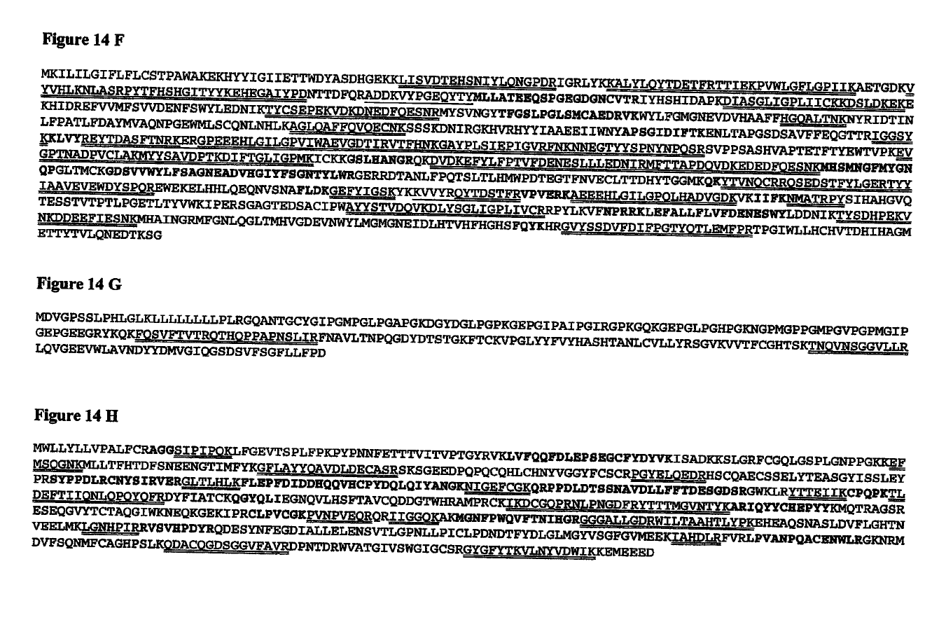

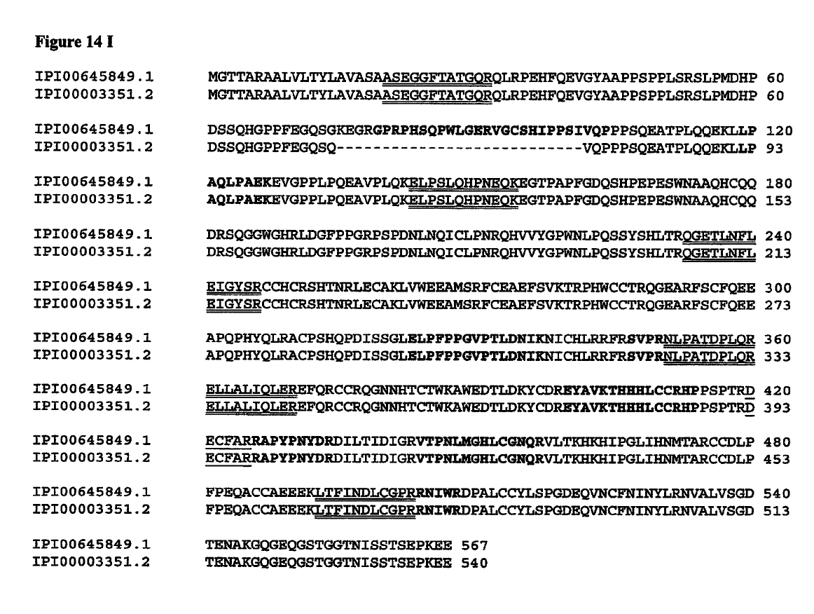

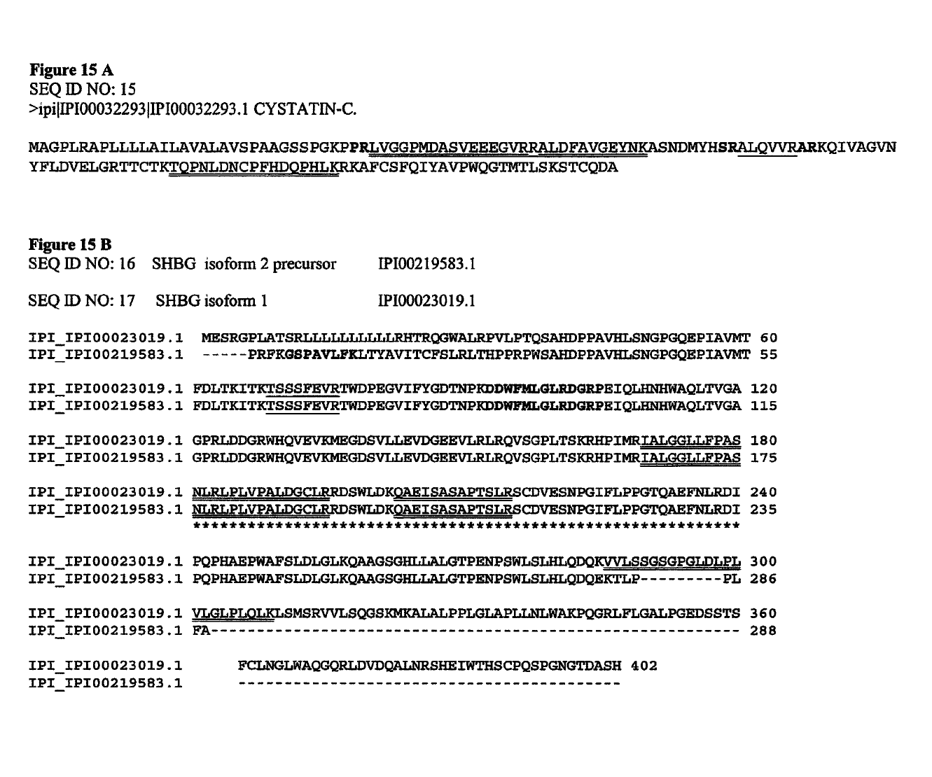

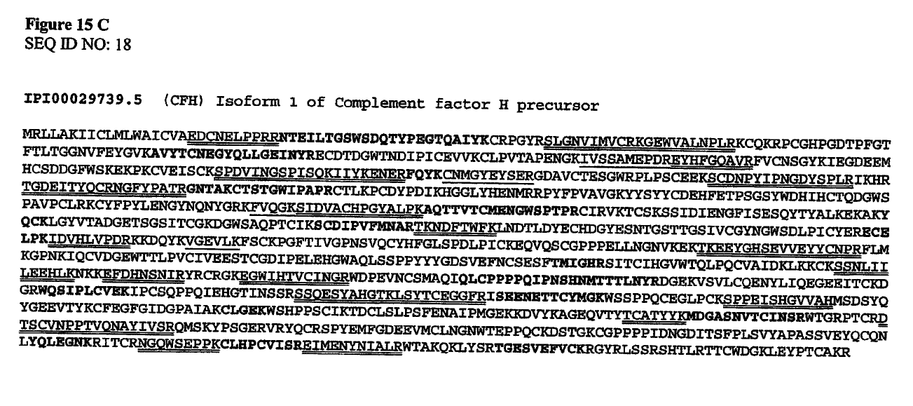

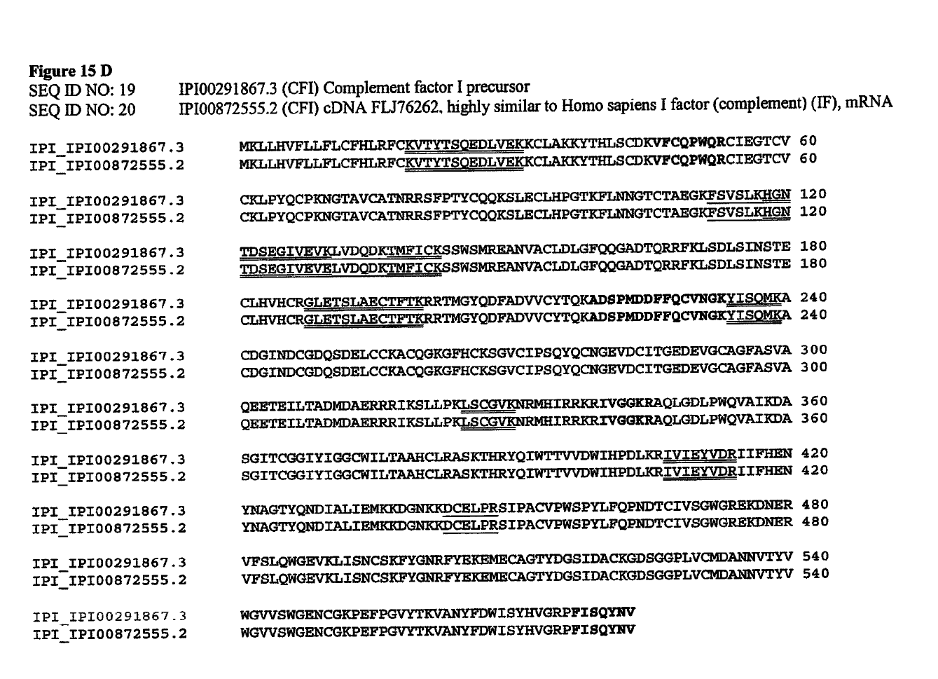

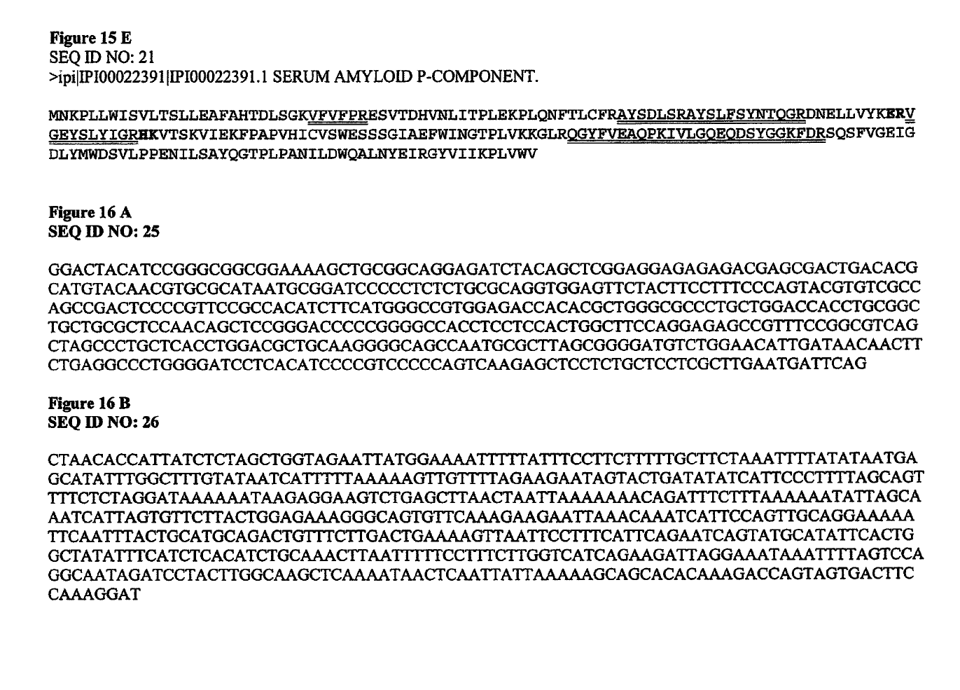

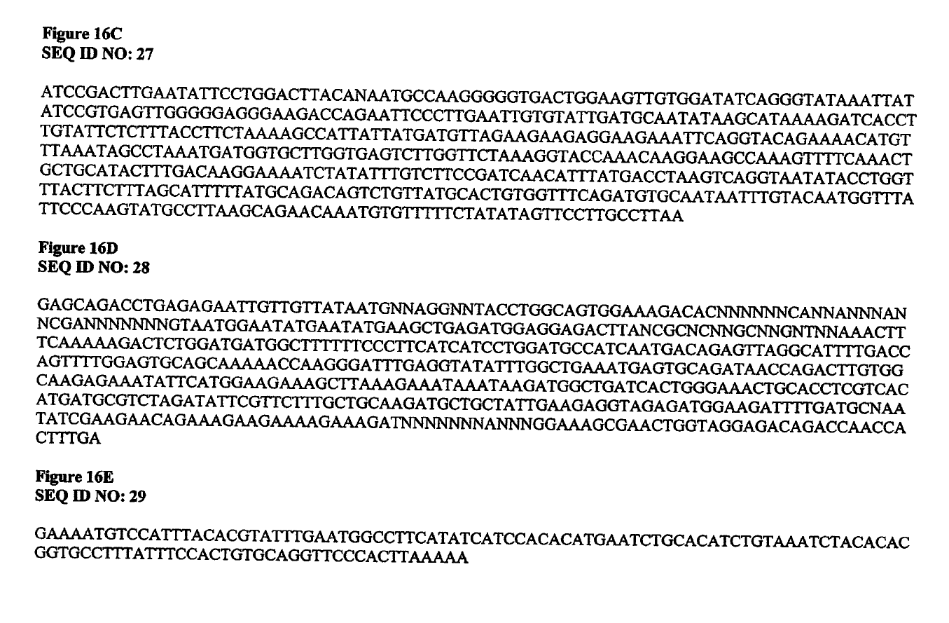

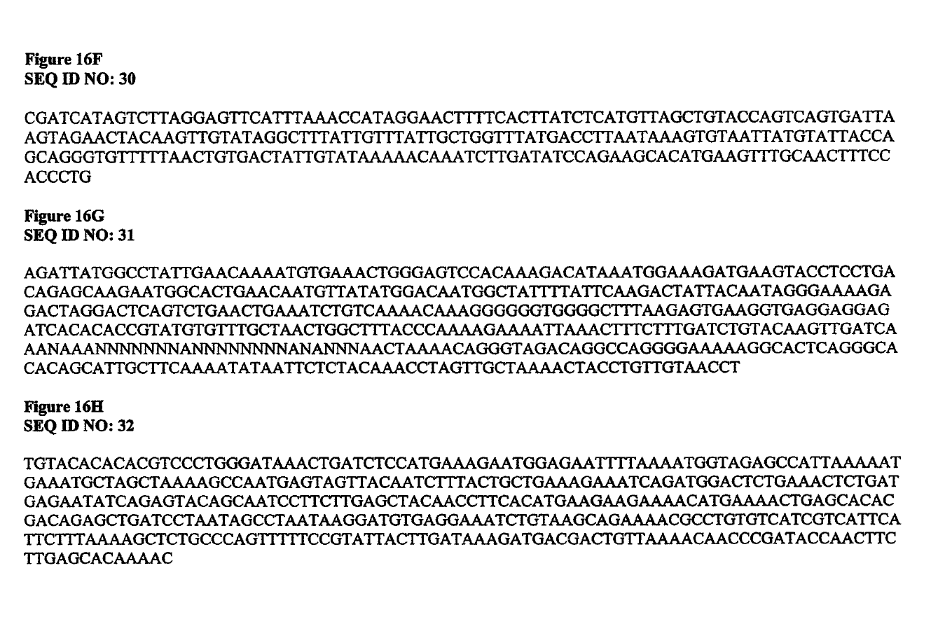

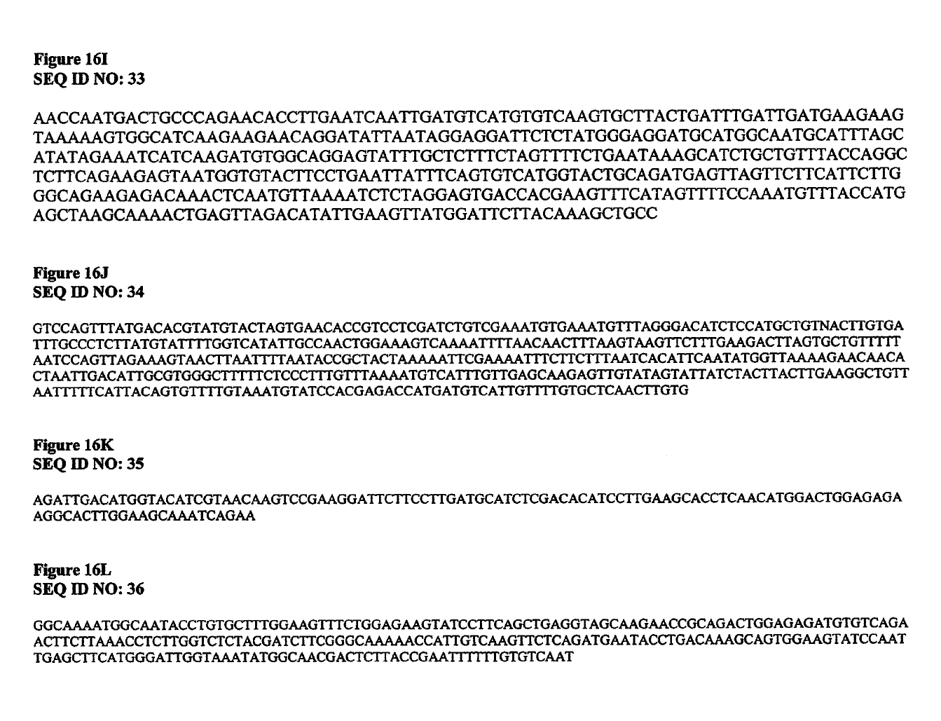

As discussed above, each of the differentially expressed probe sets demonstrated at a least 1.6-fold difference between samples from acute rejection patients (AR) and those from non-rejection patients (NR), and a subset of twelve genomic markers identified, which consistently differentiated AR and NR subjects. When Alloreactive T-cells were isolated from subject samples, and subjected to microarray analysis for identification of alloreactive T-cell genomic markers. Table 9 lists the markers demonstrating at least a 1.6 fold change. The increase or decrease in the KLF12, TTLL5, 239901_at, 241732_at, OFD1, MIRH1, WDR21A, EFCAB2, TNRC15, LENG10, MYSM1, 237060_at, C19orf59, MCL1, ANKRD25, MYL4 allowed for categorization of each sample as AR or NR (illustrated in FIG. 13A). FIG. 13b shows that the increase or decrease in alloreactive T-cell markers KLF12, TTLL5, 239901_at, 241732_at, OFD1, MIRH1, WDR21A, EFCAB2, TNRC15, LENG10, MYSM1, 237060_at, C19orf59, MCL1, ANKRD25, MYL4, when considered in combination with the increase or decrease in genomic markers TRF2, SRGAP2P1, KLF4, YLPM1, BID, MARCKS, CLEC2B, ARHGEF7, LYPLAL1, WRB, FGFR1OP2 and MBD4 markers allowed for a greater delination and better defined categorization of each sample as an AR or NR.

The above results demonstrate that specific sets of genomic markers or alloreactive T-cell genomic markers, taken alone or together, provide for a useful and consistent differentiation between subjects who are acute rejectors, or non-rejectors.

TABLE 9
Alloreactive T-cell biomarkers of acute rejection. “239901_at”, “241732_at” and “237060_at” are markers of transcripts that
do not correspond to a previously identified transcript, gene or gene product, but demonstrate statistically significant variation
between AR and NR subjects.

| | | | | | | Up or Down | |
|---|---|---|---|---|---|---|---|
| | | | | | | regulated in | |
| | | | | | | AR as | |
| | | | GenBank | Fold Change | log2 (fold | compared to | SEQ ID NO: |
| Probeset ID | Gene Symbol | Gene Name | Accession No. | (AR/NR) | change) | NR | of Target sequence |
| 206965_at | KLF12 | Kruppel-like | NM_007249 | 3.053346968 | −1.61039154 | Down | 345 |
| | | factor 12 | | | | | |
| 215898_at | TTLL5 | “tubulin tyrosine | NM_015072 | 2.970082206 | −1.570502862 | Down | 346 |
| | | ligase-like | | | | | |
| | | family, member | | | | | |
| | | 5” | | | | | |
| 239901_at | | Transcribed | — | 2.764376686 | −1.466954217 | Down | 347 |
| | | locus | | | | | |
| 241732_at | | Unknown 2 gene | — | 2.721088633 | −1.44418395 | Down | 348 |
| 241751_at | OFD1 | oral-facial- | NM_003611 | 2.68738363 | −1.426202284 | Down | 349 |
| | | digital syndrome 1 | | | | | |
| 232291_at | MIRH1 | microRNA host | XR_042147XR_042176 | 2.640301168 | −1.400702501 | Down | 350 |
| | | gene (non- | XR_042177 | | | | |
| | | protein coding) 1 | | | | | |
| 214758_at | WDR21A | WD repeat | NM_015604 | 2.46179347 | −1.299709734 | Down | 351 |
| | | domain 21A | NM_181340 | | | | |
| | | | NM_181341 | | | | |
| 1557674_s_at | EFCAB2 | EF-hand calcium | NM_032328 | 2.353831249 | −1.235010894 | Down | 352 |
| | | binding domain 2 | | | | | |
| 1560133_at | TNRC15 | trinucleotide | NM_001103146 | 2.113012444 | −1.079301264 | Down | 353 |
| | | repeat | NM_001103147 | | | | |
| | | containing 15 | NM_001103148 | | | | |
| | | | NM_015575 | | | | |
| 1564776_at | LENG10 | leukocyte | — | 1.988925732 | −0.991989406 | Down | 354 |
| | | receptor cluster | | | | | |
| | | (LRC) member | | | | | |
| | | 10 | | | | | |
| 225760_at | MYSM1 | “myb-like, | NM_001085487 | 1.67487314 | −0.744051826 | Down | 355 |
| | | SWIRM and | | | | | |
| | | MPN domains | | | | | |
| | | 1” | | | | | |
| 237060_at | | Full length insert | — | 2.27223707 | 1.184113364 | Up | 356 |
| | | cDNA clone | | | | | |
| | | ZD79D11 | | | | | |
| 235568_at | C19orf59 | chromosome 19 | NM_174918 | 2.357923591 | 1.237516968 | Up | 357 |
| | | open reading | | | | | |
| | | frame 59 | | | | | |
| 200796_s_at | MCL1 | myeloid cell | NM_021960 | 2.868955629 | 1.520525656 | Up | 358 |
| | | leukemia | NM_182763 | | | | |
| | | sequence 1 | | | | | |
| | | (BCL2-related) | | | | | |
| 218418_s_at | ANKRD25 | ankyrin repeat | NM_001136191 | 3.067259734 | 1.616950338 | Up | 359 |
| | | domain 25 | NM_015493 | | | | |
| 210088_x_at | MYL4 | “myosin, light | NM_001002841 | 3.276324766 | 1.712078372 | Up | 360 |
| | | chain 4, alkali; | NM_002476 | | | | |
| | | atrial, | | | | | |
| | | embryonic” | | | | | |

### Example 5

### Proteomic Profiling

A total of 906 protein group codes (PGC's) were detected in at least one of the 18 samples included in the discovery analysis and processed in 17 different iTRAQ experiments. Of these PGC's, 129 were detected in at least ⅔ of the 6 AR and 12 NR samples. From these two sets of PGC's, 56% and 2% were identified based on a single peptide identifier (FIG. 5). Thus, the majority of the proteins identified based on only one peptide were not identified in most of iTRAQ runs and were not further analyzed. Moreover, 57% and 40% of the 129 analyzed PGC's were identified based on >5 and >10 distinct peptides, respectively (FIG. 5).

### Discovery Analysis: Identification of Plasma Protein Markers

Statistical analysis identified 14 of the 129 analyzed PGC's whose relative concentrations differed significantly (p-value <0.05) between AR and NR samples (Table 10). Of the 14 identified PGC's, 11 were up-regulated in AR versus NR samples: B2M, F10, CP, CST3, ECMP1, CFH, C1QC, CFI, APCS, C1R and SERPINF1. The other 3 PGC's, PLTP, ADIPOQ and SHBG, were down-regulated. All PGC's were identified based on >2 distinct peptide sequences (in accordance with Paris Consensus, as per the Publication Guidelines for the Journal “Molecular and Cellular Proteomics” as of April 2007). Exemplary peptides identified in the iTRAQ experiments, the protein group code assigned and the SEQ ID NO: are listed in FIG. 17.

Panel of plasma proteins with differential relative levels between AR and NR samples (p<0.05). “PGC” contains the Protein Group Code assigned by PGCA. Accession numbers and protein names within each group, corresponding genes, p-values calculated by the robust moderated t-test (eBayes), fold changes with directions (plus and minus signs for up- or down-regulated, respectively) in AR relative to NR are given in the next five columns. Two panels were selected by a false discovery rate (FDR) criterion (A) and SDA (B) and are indicated in the last column. Panel A was selected by applying a FDR cut-off of 25%, which is equivalent to a p<0.01, on the PGCs and panel B was identified by SDA as the set of PGCs that provide the best separation between acute rejection and non-rejection samples (Table 10).

The forward selection SDA algorithm incorporates one protein group code at a time from the list of potential markers. In the first step it identifies the protein group code with the best performance based on leave-one-out cross validation. In the second step it identifies the second protein group code that, together with the previously identified code, best performs in a leave-one-out cross validation. This procedure is repeated until the improvement in the performance can not be significantly improved. In each cross-validation, performance is measured with the ability of the model to separate between acute rejection and non-rejection groups.

TABLE 10
Proteomic markers

| PGC | Accession # | Protein Name | Gene Symbol | p-value | Fold Change | Panel | SEQ ID NO: |
|---|---|---|---|---|---|---|---|
| 151 | IPI00643034.2 | Isoform 1 of Phospholipid transfer protein | PLTP | 0.0009 | −1.5 | “A, B” | 1 |
| | | precursor | | | | | |
| | IPI00217778.1 | Isoform 2 of Phospholipid transfer protein | | | | | 2 |
| | | precursor | | | | | |
| | IPI00022733.3 | 45 kDa protein | | | | | 3 |
| 92 | IPI00020019.1 | Adiponectin precursor | ADIPOQ | 0.0034 | −1.4 | “A, B” | 4 |
| 188 | IPI00868938.1 | Beta-2-microglobulin | B2M | 0.0044 | +1.5 | “A, B” | 5 |
| | IPI00004656.2 | Beta-2-microglobulin | | | | | 7 |
| | IPI00796379.1 | B2M protein | | | | | 6 |
| 84 | IPI00019576.1 | Coagulation factor X precursor | F10 | 0.0065 | +1.2 | “A, B” | 8 |
| 6 | IPI00017601.1 | Ceruloplasmin precursor | CP | 0.0086 | +1.3 | “A, B” | 9 |
| 62 | IPI00645849.1 | Extracellular matrix protein 1 | ECMP1 | 0.0217 | +1.2 | B | 10 |
| | IPI00003351.2 | Extracellular matrix protein 1 precursor | | | | | 11 |
| | IPI00479444.2 | | | | | | |
| 76 | IPI00022394.2 | Complement CIq subcomponent subunit C | CIQC | 0.0335 | +1.3 | B | 12 |
| | | precursor | | | | | |
| 26 | IPI00296165.5 | Complement C1r subcomponent precursor | C1R | 0.0483 | +1.2 | B | 13 |
| 61 | IPI00006114.4 | Pigment epithelium-derived factor precursor | SERPINF1 | 0.0483 | +1.2 | B | 14 |
| 110 | IPI00032293.1 | Cystatin-C precursor | CST3 | 0.0132 | +1.4 | | 15 |
| | IPI00301618.6 | | | | | | |
| | IPI00386885.1 | | | | | | |
| 138 | IPI00219583.1 | Isoform 2 of Sex hormone-binding globulin | SHBG | 0.0259 | −1.4 | | 16 |
| | | precursor | | | | | |
| | IPI00023019.1 | Isoform 1 of Sex hormone-binding globulin | | | | | 17 |
| | | precursor | | | | | |
| 8 | IPI00029739.5 | Isoform 1 of Complement factor H precursor | CFH | 0.0296 | +1.1 | | 18 |
| 50 | IPI00291867.3 | Complement factor I precursor | CF1 | 0.0341 | +1.2 | | 19 |
| | IPI00872555.2 | “cDNA FLJ76262, highly similar to Homo | | | | | 20 |
| | | sapiens I factor (complement) (IF), mRNA” | | | | | |
| 48 | IPI00022391.1 | Serum amyloid P-component precursor | APCS | 0.0438 | +1.1 | | 21 |

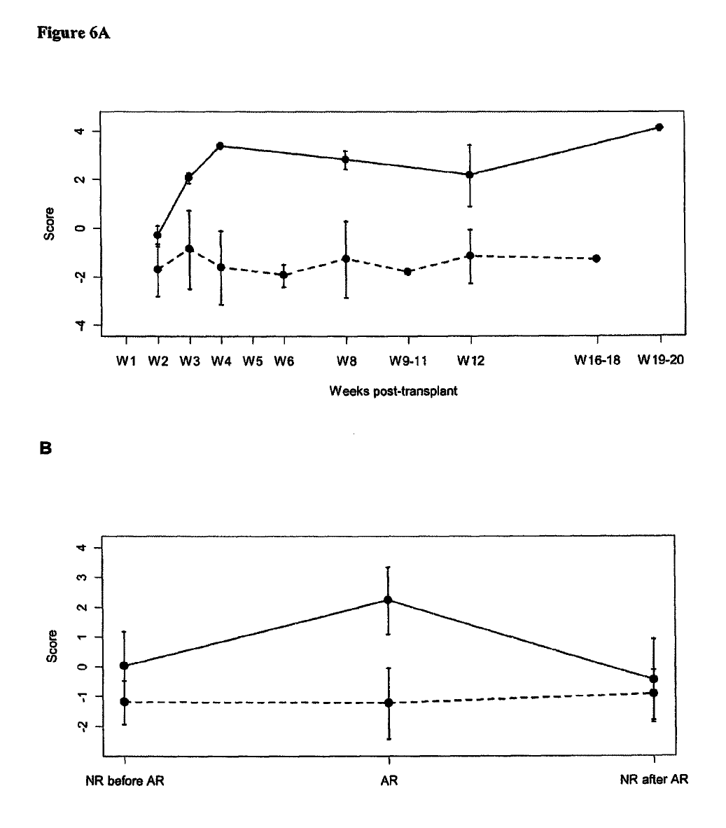

Two potential protein panels were identified based on a false discovery rate threshold (panel A) and a SDA (panel B). To visualize results across time, a single score was generated by a classifier built based on panel A using LDA (FIG. 6-A). Medians of this score for all AR (solid line) and NR (stippled line) samples available at each timepoint are displayed, and standard deviations are displayed using vertical bars (FIGS. 6A, B). Panel A clearly discriminated AR from NR at all timepoints with stronger separations after 4 weeks post-transplant. FIG. 6-B shows the score when patients transitioned between NR and AR episodes. The first consecutive timepoints of AR were considered and averaged from AR patients (solid line). Similarly, consecutive timepoints of NR before and after AR were considered and averaged from the same patients. A control curve was constructed for NR patients matched as closely as possible to AR patients by available timepoints (dashed line). Interestingly, the score for AR patients was differentially elevated at the timepoint(s) of AR compared to non-rejection states before or after acute rejection episode(s). On the contrary, NR patients presented a fairly constant pattern across matched timepoints. Similar results were obtained for the classifier built using panel B.

### Internal Validation

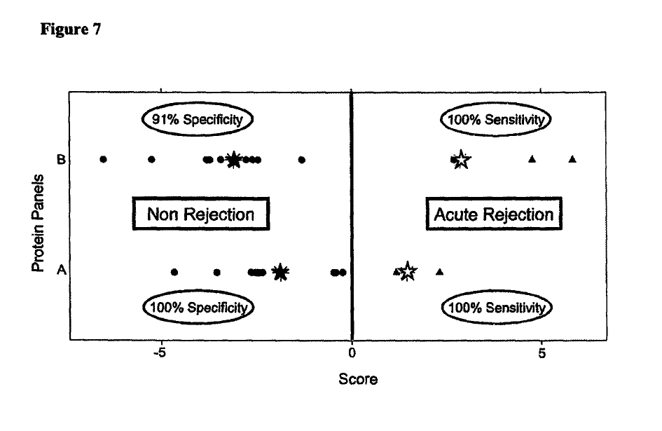

Results of an internal validation using an additional 13 patient samples using classifier A (built by LDA using panel A), and classifier B (built using panel B) are illustrated in FIG. 7. For visualization, the scores generated by both classifiers were re-centered to set the cut-off lines for classification at zero. Average scores for each of the AR and NR samples in the training set are displayed using red and black asterisks, respectively. Scores for each AR and NR samples in the test set are displayed using red triangles and black dots, respectively, showing a clear discrimination between AR and NR groups. Samples with positive values were classified as AR and those with negative values were classified as NR by LDA. Classifier A correctly classified all samples (100% sensitivity and specificity). Classifier B improved on the ability to separate the groups, but misclassified one NR sample (100% sensitivity and 91% specificity).

### Example 6

### Validation of Proteomic Expression Profile by ELISA

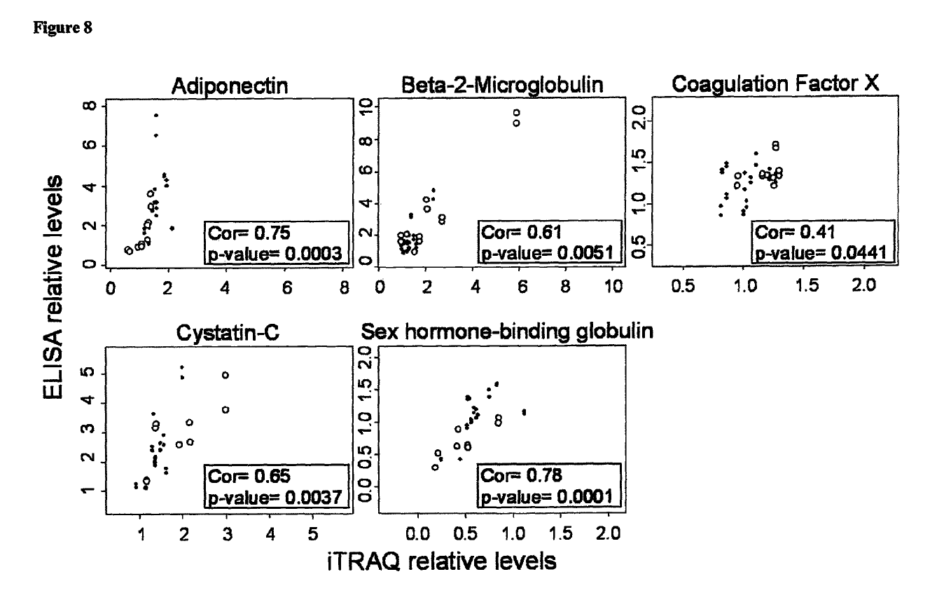

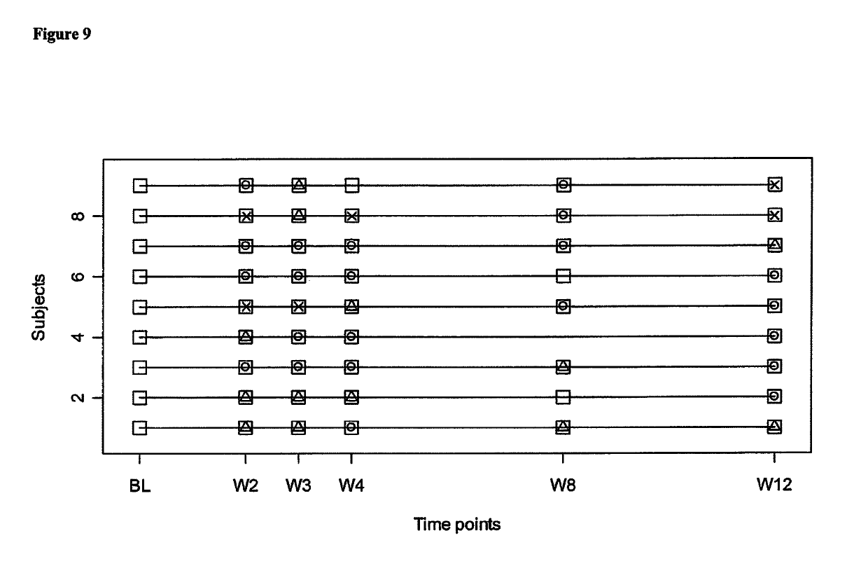

From the panel of proteins in Table 10, 5 were validated by ELISA: adiponectin, beta-2 microglobulin, cystatin C, factor X, and sex hormone-binding globulin. Although ELISA values are essentially absolute measures of protein levels, to ease comparability to the iTRAQ results, protein levels were reported relative to those of the pooled control (FIG. 8). Two important points were observed from the acquired data. First, differential protein levels between the AR and NR groups were validated. The robust moderated t-test (eBayes) was again used adjusting the correlation structure for the availability of technical duplicates in the data. Second, the correlations between ELISA and iTRAQ relative protein levels were examined. As outliers in the data can either lower the estimate of a strong correlation or inflate the estimate of a weak correlation, the Spearman correlation coefficient was used instead of the Pearson correlation coefficient.

A total of 4 out of 5 validated markers demonstrated differential protein levels in AR versus NR with p-values <0.055 (Table 11). In addition, the levels of all validated proteins were found to be in the same direction (up- and down-regulated) for AR versus NR samples in both iTRAQ and ELISA, thus corroborating the results found by iTRAQ. FIG. 8 demonstrates the correlation of protein level determined by iTRAQ (x-axis) and ELISA (y-axis) for the 18 samples used in the discovery analysis. Results provided evidence of a strong correlation between the measurements of both platforms (correlation coefficients above 0.6 and p-values from a test of positive correlation smaller than 0.006 for 4 out of 5 validated proteins). Together these results show that measurements from both platforms are well correlated.

TABLE 11
ELISA technical validation. P-values calculated by the robust
moderated t-test (eBayes), fold changes and their directions
(plus and minus signs for up- or down-regulated, respectively)
in AR relative to NR are given for each validated protein.

| Protein Name | P value | Fold change |
|---|---|---|
| SHBG | 0.0002 | −1.83 |
| ADIPOQ | 0.0014 | −2.60 |
| Cystatin-C | 0.0333 | +1.21 |
| B2M | 0.0534 | +1.64 |
| Coagulation factor X | 0.0846 | +1.05 |

All citations are herein incorporated by reference, as if each individual publication was specifically and individually indicated to be incorporated by reference herein and as though it were fully set forth herein. Citation of references herein is not to be construed nor considered as an admission that such references are prior art to the present invention.

One or more currently preferred embodiments of the invention have been described by way of example. The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

What is claimed is:

1. A method of determining the acute allograft rejection status of a subject, the method comprising the steps of:
a. determining the nucleic acid expression profile of one or more than one nucleic acid markers in a biological sample from the subject, the nucleic acid markers selected from the group comprising TRF2, SRGAP2P1, KLF4, YLPM1, BID, MARCKS, CLEC2B, ARHGEF7, LYPLAL1, WRB, FGFR1OP2 and MBD4;
b. comparing the expression profile of the one or more than one nucleic acid markers to a control profile; and
c. determining whether the expression level of the one or more than one nucleic acid markers is increased or decreased relative to the control profile;
wherein the increase or decrease of the one or more than one nucleic acid markers is indicative of the acute rejection status of the subject.

2. The method of claim 1 wherein TRF2 and FGFR1OP2 are increased relative to the non-rejector profile, and SRGAP2P1, KLF4, YLPM1, BID, MARCKS, CLEC2B, ARHGEF7, LYPLAL1, WRB, MBD4 are decreased relative to the control profile.

3. The method of claim 1 wherein the control profile is obtained from a non-rejecting, allograft recipient subject or a non-allograft recipient subject.

4. The method of claim 1, further comprising obtaining a value for one or more clinical variables.

5. The method of claim 1, further comprising at step a) determining the expression profile of one or more markers selected from Table 6.

6. The method of claim 1, wherein the nucleic acid expression profile of the one or more than one nucleic acid markers is determined by detecting an RNA sequence corresponding to one or more than one markers.

7. The method of claim 1, wherein the nucleic acid expression profile of the one or more than one nucleic acid markers is determined by PCR.

8. The method of claim 1, wherein the nucleic acid expression profile of the one or more than one nucleic acid markers is determined by hybridization.

9. The method of claim 9, wherein the hybridization is to an oligonucleotide.

10. A method of determining acute allograft rejection status of a subject, the method comprising the steps of:
a. determining a proteomic expression profile of five or more than five proteomic markers in a biological sample from the subject, the proteomic markers selected from the group comprising a polypeptide encoded by B2M, F10, CP, CST3, ECMP1, CFH, C1QC, CFI, APCS, C1R, SERPINF1, PLTP, ADIPOQ and SHBG;
b. comparing the expression profile of the five or more than five proteomic markers to a control profile; and
c. determining whether the expression level of the one or more than one proteomics markers is increased or decreased relative to the control profile;
wherein the increase or decrease of the five or more proteomic markers is indicative of the acute rejection status of the subject.

11. The method of claim 10 wherein the level of polypeptides encoded by PLTP, ADIPOQ and SHBG are decreased relative to a control, and the level of polypeptides encoded by B2M, F10, CP, CST3, ECMP1, CFH, C1QC, CFI, APCS, C1R and SERPINF1 are increased relative to a control profile.

12. The method of claim 10 wherein the control profile is obtained from a non rejecting, allograft recipient subject or a non-allograft recipient subject.

13. The method of claim 10 further comprising obtaining a value for one or more clinical variables.

14. The method of claim 10, wherein the proteomic expression profile is determined by an immunologic assay.

15. The method of claim 10, wherein the proteomic expression profile is determined by ELISA.

16. The method of claim 10, wherein the proteomic expression profile is determined by mass spectrometry.

17. The method of claim 10, wherein the proteomic expression profile is determined by an isobaric or isotope tagging method.

18. The method of claim 10 wherein the five or more than five markers include polypeptides encoded by PLTP, ADIPOQ, B2M, F10 and CP.

19. The method of claim 10 wherein the five or more than five markers include polypeptides encoded by PLTP, ADIPOQ, B2M, F10 and CP, and one or more than one of ECMP1, C1QC, C1R and SERPINF1.

20. The method of claim 1 wherein the control is an autologous control.

21. The method of claim 10 wherein the control is an autologous control.


SEQUENCE LISTING (397 sequences)

SEQ ID NO: 1 - protein, 493 aa, Homo sapiens
MALFGALFLALLAGAHAEFPGCKIRVTSKALELVKQEGLRFLEQELETITIPDLRGKEGHFYYNISEVKVTELQLTSSELDFQPQQELMLQITNASLGLRFRRQLLYWFFYDGGYINASAEGVSIRTGLELSRDPAGRMKVSNVSCQASVSRMHAAFGGTFKKVYDFLSTFITSGMRFLLNQQICPVLYHAGTVLLNSLLDTVPVRSSVDELVGIDYSLMKDPVASTSNLDMDFRGAFFPLTERNWSLPNRAVEPQLQEEERMVYVAFSEFFFDSAMESYFRAGALQLLLVGDKVPHDLDMLLRATYFGSIVLLSPAVIDSPLKLELRVLAPPRCTIKPSGTTISVTASVTIALVPPDQPEVQLSSMTMDARLSAKMALRGKALRTQLDLRRFRIYSNHSALESLALIPLQAPLKTMLQIGVMPMLNERTWRGVQIPLPEGINFVHEVVTNHAGFLTIGADLHFAKGLREVIEKNRPADVRASTAPTPSTAAV

SEQ ID NO: 2 - protein, 441 aa, Homo sapiens
MALFGALFLALLAGAHAEFPGCKIRVTSKALELVKQEGLRFLEQELETITIPDLRGKEGHFYYNISEVKVTELQLTSSELDFQPQQELMLQITNASLGLRFRRQLLYWFLKVYDFLSTFITSGMRFLLNQQICPVLYHAGTVLLNSLLDTVPVRSSVDELVGIDYSLMKDPVASTSNLDMDFRGAFFPLTERNWSLPNRAVEPQLQEEERMVYVAFSEFFFDSAMESYFRAGALQLLLVGDKVPHDLDMLLRATYFGSIVLLSPAVIDSPLKLELRVLAPPRCTIKPSGTTISVTASVTIALVPPDQPEVQLSSMTMDARLSAKMALRGKALRTQLDLRRFRIYSNHSALESLALIPLQAPLKTMLQIGVMPMLNERTWRGVQIPLPEGINFVHEVVTNHAGFLTIGADLHFAKGLREVIEKNRPADVRASTAPTPSTAAV

SEQ ID NO: 3 - protein, 405 aa, Homo sapiens
MLQITNASLGLRFRRQLLYWFFYDGGYINASAEGVSIRTGLELSRDPAGRMKVSNVSCQASVSRMHAAFGGTFKKVYDFLSTFITSGMRFLLNQQICPVLYHAGTVLLNSLLDTVPVRSSVDELVGIDYSLMKDPVASTSNLDMDFRGAFFPLTERNWSLPNRAVEPQLQEEERMVYVAFSEFFFDSAMESYFRAGALQLLLVGDKVPHDLDMLLRATYFGSIVLLSPAVIDSPLKLELRVLAPPRCTIKPSGTTISVTASVTIALVPPDQPEVQLSSMTMDARLSAKMALRGKALRTQLDLRRFRIYSNHSALESLALIPLQAPLKTMLQIGVMPMLNERTWRGVQIPLPEGINFVHEVVTNHAGFLTIGADLHFAKGLREVIEKNRPADVRASTAPTPSTAAV

SEQ ID NO: 4 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 5 - protein, 101 aa, Homo sapiens
MSRSVALAVLALLSLSGLEAIQRTPKIQVYSRHPSDIEVDLLKNGERIEKVEHSDLSFSKDWSFYLLYYTEFTPTEKDEYACRVNHVTLSQPKIVKWDRDM

SEQ ID NO: 6 - protein, 119 aa, Homo sapiens [sequence omitted]

SEQ ID NO: 7 - protein, 124 aa, Homo sapiens [sequence omitted]

SEQ ID NO: 8 - protein, 488 aa, Homo sapiens
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 9 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 10 - protein, 567 aa, Homo sapiens
MGTTARAALVLTYLAVASAASEGGFTATGQRQLRPEHFQEVGYAAPPSPPLSRSLPMDHPDSSQHGPPFEGQSGKEGRGPRPHSQPWLGERVGCSHIPPSIVQPPPSQEATPLQQEKLLPAQLPAEKEVGPPLPQEAVPLQKELPSLQHPNEQKEGTPAPFGDQSHPEPESWNAAQHCQQDRSQGGWGHRLDGFPPGRPSPDNLNQICLPNRQHVVYGPWNLPQSSYSHLTRQGETLNFLEIGYSRCCHCRSHTNRLECAKLVWEEAMSRFCEAEFSVKTRPHWCCTRQGEARFSCFQEEAPQPHYQLRACPSHQPDISSGLELPFPPGVPTLDNIKNICHLRRFRSVPRNLPATDPLQRELLALIQLEREFQRCCRQGNNHTCTWKAWEDTLDKYCDREYAVKTHHHLCCRHPPSPTRDECFARRAPYPNYDRDILTIDIGRVTPNLMGHLCGNQRVLTKHKHIPGLIHNMTARCCDLPFPEQACCAEEEKLTFINDLCGPRRNIWRDPALCCYLSPGDEQVNCFNINYLRNVALVSGDTENAKGQGEQGSTGGTNISSTSEPKEE

SEQ ID NO: 11 - protein, 540 aa, Homo sapiens
MGTTARAALVLTYLAVASAASEGGFTATGQRQLRPEHFQEVGYAAPPSPPLSRSLPMDHPDSSQHGPPFEGQSQVQPPPSQEATPLQQEKLLPAQLPAEKEVGPPLPQEAVPLQKELPSLQHPNEQKEGTPAPFGDQSHPEPESWNAAQHCQQDRSQGGWGHRLDGFPPGRPSPDNLNQICLPNRQHVVYGPWNLPQSSYSHLTRQGETLNFLEIGYSRCCHCRSHTNRLECAKLVWEEAMSRFCEAEFSVKTRPHWCCTRQGEARFSCFQEEAPQPHYQLRACPSHQPDISSGLELPFPPGVPTLDNIKNICHLRRFRSVPRNLPATDPLQRELLALIQLEREFQRCCRQGNNHTCTWKAWEDTLDKYCDREYAVKTHHHLCCRHPPSPTRDECFARRAPYPNYDRDILTIDIGRVTPNLMGHLCGNQRVLTKHKHIPGLIHNMTARCCDLPFPEQACCAEEEKLTFINDLCGPRRNIWRDPALCCYLSPGDEQVNCFNINYLRNVALVSGDTENAKGQGEQGSTGGTNISSTSEPKEE

SEQ ID NO: 12 - protein, 245 aa, Homo sapiens
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 13 - protein, 705 aa, Homo sapiens
MWLLYLLVPALFCRAGGSIPIPQKLFGEVTSPLFPKPYPNNFETTTVITVPTGYRVKLVFQQFDLEPSEGCFYDYVKISADKKSLGRFCGQLGSPLGNPPGKKEFMSQGNKMLLTFHTDFSNEENGTIMFYKGFLAYYQAVDLDECASRSKSGEEDPQPQCQHLCHNYVGGYFCSCRPGYELQEDRHSCQAECSSELYTEASGYISSLEYPRSYPPDLRCNYSIRVERGLTLHLKFLEPFDIDDHQQVHCPYDQLQIYANGKNIGEFCGKQRPPDLDTSSNAVDLLFFTDESGDSRGWKLRYTTEIIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 14 - protein, 418 aa, Homo sapiens [sequence omitted]

SEQ ID NO: 15 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 16 - protein, 288 aa, Homo sapiens
PRFKGSPAVLFKLTYAVITCFSLRLTHPPRPWSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQEKTLPPLFA

SEQ ID NO: 17 - protein, 402 aa, Homo sapiens
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQKVVLSSGSGPGLDLPLVLGLPLQLKLSMSRVVLSQGSKMKALALPPLGLAPLLNLWAKPQGRLFLGALPGEDSSTSFCLNGLWAQGQRLDVDQALNRSHEIWTHSCPQSPGNGTDASH

SEQ ID NO: 18 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 19 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVAQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 20 - protein, 583 aa, Homo sapiens [sequence omitted]

SEQ ID NO: 21 - protein, 223 aa, Homo sapiens
MNKPLLWISVLTSLLEAFAHTDLSGKVFVFPRESVTDHVNLITPLEKPLQNFTLCFRAYSDLSRAYSLFSYNTQGRDNELLVYKERVGEYSLYIGRHKVTSKVIEKFPAPVHICVSWESSSGIAEFWINGTPLVKKGLRQGYFVEAQPKIVLGQEQDSYGGKFDRSQSFVGEIGDLYMWDSVLPPENILSAYQGTPLPANILDWQALNYEIRGYVIIKPLVWV

SEQ ID NO: 22-24 - skipped (3 entries)

SEQ ID NO: 25 - DNA, 457 bp, Homo sapiens
ggactacatccgggcggcggaaaagctgcggcaggagatctacagctcggaggagagagacgagcgactgacacgcatgtacaacgtgcgcataatgcggatccccctctctgcgcaggtggagttctacttcctttcccagtacgtgtcgccagccgactccccgttccgccacatcttcatgggccgtggagaccacacgctgggcgccctgctggaccacctgcggctgctgcgctccaacagctccgggacccccggggccacctcctccactggcttccaggagagccgtttccggcgtcagctagccctgctcacctggacgctgcaaggggcagccaatgcgcttagcggggatgtctggaacattgataacaacttctgaggccctggggatcctcacatccccgtcccccagtcaagagctcctctgctcctcgcttgaatgattcag

SEQ ID NO: 26 - DNA, 552 bp, Homo sapiens
ctaacaccattatctctagctggtagaattatggaaaatttttatttccttctttttgcttctaaattttatataatgagcatatttggctttgtataatcatttttaaaaagttgttttagaagaatagtactgatatatcattcccttttagcagttttctctaggataaaaaataagaggaagtctgagcttaactaattaaaaaaacagatttctttaaaaaatattagcaaatcattagtgttcttactggagaaagggcagtgttcaaagaagaattaaacaaatcattccagttgcaggaaaaattcaatttactgcatgcagactgtttcttgactgaaaagttaattcctttcattcagaatcagtatgcatattcactggctatatttcatctcacatctgcaaacttaatttttcctttcttggtcatcagaagattaggaaataaattttagtccaggcaatagatcctacttggcaagctcaaaataactcaattattaaaaagcagcacacaaagaccagtagtgacttccaaaggat

SEQ ID NO: 27 - DNA, 523 bp, Homo sapiens
  modified_base (29)..(29): a, c, t, or g
atccgacttgaatattcctggacttacanaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaa

SEQ ID NO: 28 - DNA, 537 bp, Homo sapiens
  modified_base (32)..(33): a, c, t, or g
  modified_base (37)..(38): a, c, t, or g
  modified_base (60)..(65): a, c, t, or g
  modified_base (68)..(69): a, c, t, or g
  modified_base (71)..(73): a, c, t, or g
  modified_base (75)..(76): a, c, t, or g
  modified_base (80)..(86): a, c, t, or g
  modified_base (128)..(128): a, c, t, or g
  modified_base (132)..(132): a, c, t, or g
  modified_base (134)..(135): a, c, t, or g
  modified_base (138)..(139): a, c, t, or g
  modified_base (141)..(141): a, c, t, or g
  modified_base (143)..(144): a, c, t, or g
  modified_base (455)..(455): a, c, t, or g
  modified_base (489)..(495): a, c, t, or g
  modified_base (497)..(499): a, c, t, or g
gagcagacctgagagaattgttgttataatgnnaggnntacctggcagtggaaagacacnnnnnncannannnanncgannnnnnngtaatggaatatgaatatgaagctgagatggaggagacttancgcncnngcnngntnnaaactttcaaaaagactctggatgatggcttttttcccttcatcatcctggatgccatcaatgacagagttaggcattttgaccagttttggagtgcagcaaaaaccaagggatttgaggtatatttggctgaaatgagtgcagataaccagacttgtggcaagagaaatattcatggaagaaagcttaaagaaataaataagatggctgatcactgggaaactgcacctcgtcacatgatgcgtctagatattcgttctttgctgcaagatgctgctattgaagaggtagagatggaagattttgatgcnaatatcgaagaacagaaagaagaaaagaaagatnnnnnnnannnggaaagcgaactggtaggagacagaccaaccactttga

SEQ ID NO: 29 - DNA, 117 bp, Homo sapiens
gaaaatgtccatttacacgtatttgaatggccttcatatcatccacacatgaatctgcacatctgtaaatctacacacggtgcctttatttccactgtgcaggttcccacttaaaaa

SEQ ID NO: 30 - DNA, 239 bp, Homo sapiens
cgatcatagtcttaggagttcatttaaaccataggaacttttcacttatctcatgttagctgtaccagtcagtgattaagtagaactacaagttgtataggctttattgtttattgctggtttatgaccttaataaagtgtaattatgtattaccagcagggtgtttttaactgtgactattgtataaaaacaaatcttgatatccagaagcacatgaagtttgcaactttccaccctg

SEQ ID NO: 31 - DNA, 443 bp, Homo sapiens
  modified_base (308)..(308): a, c, t, or g
  modified_base (312)..(318): a, c, t, or g
  modified_base (320)..(327): a, c, t, or g
  modified_base (329)..(329): a, c, t, or g
  modified_base (331)..(333): a, c, t, or g
agattatggcctattgaacaaaatgtgaaactgggagtccacaaagacataaatggaaagatgaagtacctcctgacagagcaagaatggcactgaacaatgttatatggacaatggctattttattcaagactattacaatagggaaaagagactaggactcagtctgaactgaaatctgtcaaaacaaaggggggtggggctttaagagtgaaggtgaggaggagatcacacaccgtatgtgtttgctaactggctttacccaaaagaaaattaaactttctttgatctgtacaagttgatcaaanaaannnnnnnannnnnnnnanannnaactaaaacagggtagacaggccaggggaaaaaggcactcagggcacacagcattgcttcaaaatataattctctacaaacctagttgctaaaactacctgttgtaacct

SEQ ID NO: 32 - DNA, 398 bp, Homo sapiens
tgtacacacacgtccctgggataaactgatctccatgaaagaatggagaattttaaaatggtagagccattaaaaatgaaatgctagctaaaagccaatgagtagttacaatctttactgctgaaagaaatcagatggactctgaaactctgatgagaatatcagagtacagcaatccttcttgagctacaaccttcacatgaagaagaaaacatgaaaactgagcacacgacagagctgatcctaatagcctaataaggatgtgaggaaatctgtaagcagaaaacgcctgtgtcatcgtcattcattctttaaaagctctgcccagtttttccgtattacttgataaagatgacgactgttaaaacaacccgataccaacttcttgagcacaaaac

SEQ ID NO: 33 - DNA, 441 bp, Homo sapiens
aaccaatgactgcccagaacaccttgaatcaattgatgtcatgtgtcaagtgcttactgatttgattgatgaagaagtaaaaagtggcatcaagaagaacaggatattaataggaggattctctatgggaggatgcatggcaatgcatttagcatatagaaatcatcaagatgtggcaggagtatttgctctttctagttttctgaataaagcatctgctgtttaccaggctcttcagaagagtaatggtgtacttcctgaattatttcagtgtcatggtactgcagatgagttagttcttcattcttgggcagaagagacaaactcaatgttaaaatctctaggagtgaccacgaagtttcatagttttccaaatgtttaccatgagctaagcaaaactgagttagacatattgaagttatggattcttacaaagctgcc

SEQ ID NO: 34 - DNA, 446 bp, Homo sapiens
  modified_base (85)..(85): a, c, t, or g
gtccagtttatgacacgtatgtactagtgaacaccgtcctcgatctgtcgaaatgtgaaatgtttagggacatctccatgctgtnacttgtgatttgccctcttatgtattttggtcatattgccaactggaaagtcaaaattttaacaactttaagtaagttctttgaagacttagtgctgtttttaatccagttagaaagtaacttaattttaataccgctactaaaaattcgaaaatttcttctttaatcacattcaatatggttaaaagaacaacactaattgacattgcgtgggctttttctccctttgtttaaaatgtcatttgttgagcaagagttgtatagtattatctacttacttgaaggctgttaatttttcattacagtgttttgtaaatgtatccacgagaccatgatgtcattgttttgtgctcaacttgtg

SEQ ID NO: 35 - DNA, 115 bp, Homo sapiens
agattgacatggtacatcgtaacaagtccgaaggattcttccttgatgcatctcgacacatccttgaagcacctcaacatggactggagagaaggcacttggaagcaaatcagaa

SEQ ID NO: 36 - DNA, 243 bp, Homo sapiens
ggcaaaatggcaatacctgtgctttggaagtttctggagaagtatccttcagctgaggtagcaagaaccgcagactggagagatgtgtcagaacttcttaaacctcttggtctctacgatcttcgggcaaaaaccattgtcaagttctcagatgaatacctgacaaagcagtggaagtatccaattgagcttcatgggattggtaaatatggcaacgactcttaccgaattttttgtgtcaat

SEQ ID NO: 37 - protein, 12 aa, Artificial Sequence
  Description of Artificial Sequence: Synthetic peptide
  Mass spectrometry peptide #37
AAFFHGQALTNK

SEQ ID NO: 38 - protein, 13 aa, Artificial Sequence
  Description of Artificial Sequence: Synthetic peptide
  Mass spectrometry peptide No. 38
ADDKVYPGEQYTY

SEQ ID NO: 39 - protein, 33 aa, Artificial Sequence
  Description of Artificial Sequence: Synthetic polypeptide
  Mass spectrometry peptide #39
ADDKVYPGEQYTYMLLATEEQSPGEGDGNCVTR

SEQ ID NO: 40 - protein, 20 aa, Artificial Sequence
  Description of Artificial Sequence: Synthetic peptide
  Mass spectrometry peptide # 40
AEEEHLGILGPQLHADVGDK

SEQ ID NO: 41 - protein, 6 aa, Artificial Sequence
  Description of Artificial Sequence: Synthetic peptide
  Mass spectrometry peptide #41
AETGDK

SEQ ID NO: 42 - protein, 14 aa, Artificial Sequence
  Description of Artificial Sequence: Synthetic peptide
  Mass spectrometry peptide #42
AGLQAFFQVQECNK

SEQ ID NO: 43 - protein, 12 aa, Artificial Sequence
  Description of Artificial Sequence: Synthetic peptide
  Mass spectrometry peptide #43
ALYLQYTDETFR

SEQ ID NO: 44 - protein, 15 aa, Artificial Sequence
  Description of Artificial Sequence: Synthetic peptide
  Mass spectrometry peptide #44
ALYLQYTDETFRTTI

SEQ ID NO: 45 - protein, 7 aa, Artificial Sequence
  Description of Artificial Sequence: Synthetic peptide
  Mass spectrometry peptide #45
ASDHGEK

SEQ ID NO: 46 - protein, 17 aa, Artificial Sequence
  Description of Artificial Sequence: Synthetic peptide
  Mass spectrometry peptide #46
ATEEQSPGEGDGNCVTR

SEQ ID NO: 47 - protein, 10 aa, Artificial Sequence
  Description of Artificial Sequence: Synthetic peptide
  Mass spectrometry peptide #47
AYYSTVDQVK

SEQ ID NO: 48 - protein, 19 aa, Artificial Sequence
  Description of Artificial Sequence: Synthetic peptide
  Mass spectrometry peptide #48
CSEPEKVDKDNEDFQESNR

SEQ ID NO: 49 - protein, 10 aa, Artificial Sequence
  Description of Artificial Sequence: Synthetic peptide
  Mass spectrometry peptide #49
DDEEFIESNK

SEQ ID NO: 50 - protein, 14 aa, Artificial Sequence
  Description of Artificial Sequence: Synthetic peptide
  Mass spectrometry peptide #50
DIASGLIGPLIICK

SEQ ID NO: 51 - protein, 15 aa, Artificial Sequence
  Description of Artificial Sequence: Synthetic peptide
  Mass spectrometry peptide #51
DIASGLIGPLIICKK

SEQ ID NO: 52 - protein, 11 aa, Artificial Sequence
  Description of Artificial Sequence: Synthetic peptide
  Mass spectrometry peptide #52
DIFTGLIGPMK

SEQ ID NO: 53 - protein, 14 aa, Artificial Sequence
  Description of Artificial Sequence: Synthetic peptide
  Mass spectrometry peptide #53
DLYSGLIGPLIVCR

SEQ ID NO: 54 - protein, 10 aa, Artificial Sequence
  Description of Artificial Sequence: Synthetic peptide
  Mass spectrometry peptide #54
DNEDFQESNR

SEQ ID NO: 55 - protein, 7 aa, Artificial Sequence
  Description of Artificial Sequence: Synthetic peptide
  Mass spectrometry peptide #55
DSLDKEK

SEQ ID NO: 56 - protein, 14 aa, Artificial Sequence
  Description of Artificial Sequence: Synthetic peptide
  Mass spectrometry peptide #56
DTANLFPQTSLTLH

SEQ ID NO: 57 - protein, 26 aa, Artificial Sequence
  Description of Artificial Sequence: Synthetic peptide
  Mass spectrometry peptide #57
DVDKEFYLFPTVFDENESLLLEDNIR

SEQ ID NO: 58 - protein, 10 aa, Artificial Sequence
  Description of Artificial Sequence: Synthetic peptide
  Mass spectrometry peptide #58
EDEDFQESNK

SEQ ID NO: 59 - protein, 8 aa, Artificial Sequence
  Description of Artificial Sequence: Synthetic peptide
  Mass spectrometry peptide #59
EDFQESNR

SEQ ID NO: 60 - protein, 21 aa, Artificial Sequence
  Description of Artificial Sequence: Synthetic peptide
  Mass spectrometry peptide #60
EEEHLGILGPVIWAEVGDTIR

SEQ ID NO: 61 - protein, 14 aa, Artificial Sequence
  Description of Artificial Sequence: Synthetic peptide
  Mass spectrometry peptide #61
EGTYYSPNYNPQSR

SEQ ID NO: 62 - protein, 9 aa, Artificial Sequence
  Description of Artificial Sequence: Synthetic peptide
  Mass spectrometry peptide #62
EHEGAIYPD

SEQ ID NO: 63 - protein, 16 aa, Artificial Sequence
  Description of Artificial Sequence: Synthetic peptide
  Mass spectrometry peptide #63
EHEGAIYPDNTTDFQR

SEQ ID NO: 64 - protein, 13 aa, Artificial Sequence
  Description of Artificial Sequence: Synthetic peptide
  Mass spectrometry peptide #64
EHSNIYLQNGPDR

SEQ ID NO: 65 - protein, 25 aa, Artificial Sequence
  Description of Artificial Sequence: Synthetic peptide
  Mass spectrometry peptide #65
ERGPEEEHLGILGPVIWAEVGDTIR

SEQ ID NO: 66 - protein, 14 aa, Artificial Sequence
  Description of Artificial Sequence: Synthetic peptide
  Mass spectrometry peptide #66
EVGPTNADPVCLAK

SEQ ID NO: 67 - protein, 10 aa, Artificial Sequence
  Description of Artificial Sequence: Synthetic peptide
  Mass spectrometry peptide #67
EYTDASFTNR

SEQ ID NO: 68 - protein, 11 aa, Artificial Sequence
  Description of Artificial Sequence: Synthetic peptide
  Mass spectrometry peptide #68
EYTDASFTNRK

SEQ ID NO: 69 - protein, 7 aa, Artificial Sequence
  Description of Artificial Sequence: Synthetic peptide
  Mass spectrometry peptide #69
FLGPIIK

SEQ ID NO: 70 - protein, 13 aa, Artificial Sequence
  Description of Artificial Sequence: Synthetic peptide
  Mass spectrometry peptide #70
FNKNNEGTYYSPN

SEQ ID NO: 71 - protein, 15 aa, Artificial Sequence
  Description of Artificial Sequence: Synthetic peptide
  Mass spectrometry peptide #71
FNKNNEGTYYSPNYN

SEQ ID NO: 72 - protein, 19 aa, Artificial Sequence
  Description of Artificial Sequence: Synthetic peptide
  Mass spectrometry peptide #72
FNKNNEGTYYSPNYNPQSR

SEQ ID NO: 73 - protein, 20 aa, Artificial Sequence
  Description of Artificial Sequence: Synthetic peptide
  Mass spectrometry peptide #73
FTTAPDQVDKEDEDFQESNK

SEQ ID NO: 74 - protein, 13 aa, Artificial Sequence
  Description of Artificial Sequence: Synthetic peptide
  Mass spectrometry peptide #74
GAYPLSIEPIGVR

SEQ ID NO: 75 - protein, 8 aa, Artificial Sequence
  Description of Artificial Sequence: Synthetic peptide
  Mass spectrometry peptide #75
GEFYIGSK

SEQ ID NO: 76 - protein, 8 aa, Artificial Sequence
  Description of Artificial Sequence: Synthetic peptide
  Mass spectrometry peptide #76
GFLGPIIK

SEQ ID NO: 77 - protein, 7 aa, Artificial Sequence
  Description of Artificial Sequence: Synthetic peptide
  Mass spectrometry peptide #77
GHSFQYK

SEQ ID NO: 78 - protein, 6 aa, Artificial Sequence
  Description of Artificial Sequence: Synthetic peptide
  Mass spectrometry peptide #78
GITYYK

SEQ ID NO: 79 - protein, 23 aa, Artificial Sequence
  Description of Artificial Sequence: Synthetic peptide
  Mass spectrometry peptide #79
GPEEEHLGILGPVIWAEVGDTIR

SEQ ID NO: 80 - protein, 8 aa, Artificial Sequence
  Description of Artificial Sequence: Synthetic peptide
  Mass spectrometry peptide #80
GSLHANGR

SEQ ID NO: 81 - protein, 13 aa, Artificial Sequence
  Description of Artificial Sequence: Synthetic peptide
  Mass spectrometry peptide #81
GTYYSPNYNPQSR

SEQ ID NO: 82 - protein, 9 aa, Artificial Sequence
  Description of Artificial Sequence: Synthetic peptide
  Mass spectrometry peptide #82
GVYSSDVFD

SEQ ID NO: 83 - protein, 23 aa, Artificial Sequence
  Description of Artificial Sequence: Synthetic peptide
  Mass spectrometry peptide #83
GVYSSDVFDIFPGTYQTLEMFPR

SEQ ID NO: 84 - protein, 8 aa, Artificial Sequence
  Description of Artificial Sequence: Synthetic peptide
  Mass spectrometry peptide #84
HGQALTNK

SEQ ID NO: 85 - protein, 10 aa, Artificial Sequence
  Description of Artificial Sequence: Synthetic peptide
  Mass spectrometry peptide #85
HIDREFVVMF

SEQ ID NO: 86 - protein, 14 aa, Artificial Sequence
  Description of Artificial Sequence: Synthetic peptide
  Mass spectrometry peptide #86
IAAVEVEWDYSPQR

SEQ ID NO: 87 - protein, 6 aa, Artificial Sequence
  Description of Artificial Sequence: Synthetic peptide
  Mass spectrometry peptide #87
IGGSYK

SEQ ID NO: 88 - protein, 8 aa, Artificial Sequence
  Description of Artificial Sequence: Synthetic peptide
  Mass spectrometry peptide #88
IGPLIVCR

SEQ ID NO: 89 - protein, 10 aa, Artificial Sequence
  Description of Artificial Sequence: Synthetic peptide
  Mass spectrometry peptide #89
IYHSHIDAPK

SEQ ID NO: 90 - protein, 9 aa, Artificial Sequence
  Description of Artificial Sequence: Synthetic peptide
  Mass spectrometry peptide #90
IYLQNGPDR

SEQ ID NO: 91 - protein, 13 aa, Artificial Sequence
  Description of Artificial Sequence: Synthetic peptide
  Mass spectrometry peptide #91
KALYLQYTDETFR

SEQ ID NO: 92 - protein, 11 aa, Artificial Sequence
  Description of Artificial Sequence: Synthetic peptide
  Mass spectrometry peptide #92
KDDEEFIESNK

SEQ ID NO: 93 - protein, 7 aa, Artificial Sequence
  Description of Artificial Sequence: Synthetic peptide
  Mass spectrometry peptide #93
KLEFALL

SEQ ID NO: 94 - protein, 8 aa, Artificial Sequence
  Description of Artificial Sequence: Synthetic peptide
  Mass spectrometry peptide #94
KLEFALLF

SEQ ID NO: 95 - protein, 20 aa, Artificial Sequence
  Description of Artificial Sequence: Synthetic peptide
  Mass spectrometry peptide #95
KLISVDTEHSNIYLQNGPDR

SEQ ID NO: 96 - protein, 17 aa, Artificial Sequence
  Description of Artificial Sequence: Synthetic peptide
  Mass spectrometry peptide #96
KNNEGTYYSPNYNPQSR

SEQ ID NO: 97 - protein, 5 aa, Artificial Sequence
  Description of Artificial Sequence: Synthetic peptide
  Mass spectrometry peptide #97
KVVYR

SEQ ID NO: 98 - protein, 9 aa, Artificial Sequence
  Description of Artificial Sequence: Synthetic peptide
  Mass spectrometry peptide #98
LGFLGPIIK

SEQ ID NO: 99 - protein, 19 aa, Artificial Sequence
  Description of Artificial Sequence: Synthetic peptide
  Mass spectrometry peptide #99
LISVDTEHSNIYLQNGPDR

SEQ ID NO: 100 - protein, 19 aa, Artificial Sequence
  Description of Artificial Sequence: Synthetic peptide
  Mass spectrometry peptide #100
LLATEEQSPGEGDGNCVTR

SEQ ID NO: 101 - protein, 11 aa, Artificial Sequence
  Description of Artificial Sequence: Synthetic peptide
  Mass spectrometry peptide #101
MFTTAPDQVDK

SEQ ID NO: 102 - protein, 13 aa, Artificial Sequence
  Description of Artificial Sequence: Synthetic peptide
  Mass spectrometry peptide #102
MFTTAPDQVDKED

SEQ ID NO: 103 - protein, 21 aa, Artificial Sequence
  Description of Artificial Sequence: Synthetic peptide
  Mass spectrometry peptide #103
MFTTAPDQVDKEDEDFQESNK

SEQ ID NO: 104 - protein, 7 aa, Artificial Sequence
  Description of Artificial Sequence: Synthetic peptide
  Mass spectrometry peptide #104
MHAINGR

SEQ ID NO: 105 - protein, 19 aa, Artificial Sequence
  Description of Artificial Sequence: Synthetic peptide
  Mass spectrometry peptide #105
MHSMNGFMYGNQPGLTMCK

SEQ ID NO: 106 - protein, 20 aa, Artificial Sequence
  Description of Artificial Sequence: Synthetic peptide
  Mass spectrometry peptide #106
MLLATEEQSPGEGDGNCVTR

SEQ ID NO: 107 - protein, 22 aa, Artificial Sequence
  Description of Artificial Sequence: Synthetic peptide
  Mass spectrometry peptide #107
MYSVNGYTFGSLPGLSMCAEDR

SEQ ID NO: 108 - protein, 24 aa, Artificial Sequence
  Description of Artificial Sequence: Synthetic peptide
  Mass spectrometry peptide #108
MYSVNGYTFGSLPGLSMCAEDRVK

SEQ ID NO: 109 - protein, 10 aa, Artificial Sequence
  Description of Artificial Sequence: Synthetic peptide
  Mass spectrometry peptide #109
MYYSAVDPTK

SEQ ID NO: 110 - protein, 21 aa, Artificial Sequence
  Description of Artificial Sequence: Synthetic peptide
  Mass spectrometry peptide #110
MYYSAVDPTKDIFTGLIGPMK

SEQ ID NO: 111 - protein, 15 aa, Artificial Sequence
  Description of Artificial Sequence: Synthetic peptide
  Mass spectrometry peptide #111
NEGTYYSPNYNPQSR

SEQ ID NO: 112 - protein, 18 aa, Artificial Sequence
  Description of Artificial Sequence: Synthetic peptide
  Mass spectrometry peptide #112
NKNNEGTYYSPNYNPQSR

SEQ ID NO: 113 - protein, 9 aa, Artificial Sequence
  Description of Artificial Sequence: Synthetic peptide
  Mass spectrometry peptide #113
NLASRPYTF

SEQ ID NO: 114 - protein, 10 aa, Artificial Sequence
  Description of Artificial Sequence: Synthetic peptide
  Mass spectrometry peptide #114
NLASRPYTFH

SEQ ID NO: 115 - protein, 7 aa, Artificial Sequence
  Description of Artificial Sequence: Synthetic peptide
  Mass spectrometry peptide #115
NMATRPY

SEQ ID NO: 116 - protein, 10 aa, Artificial Sequence
  Description of Artificial Sequence: Synthetic peptide
  Mass spectrometry peptide #116
NNEGTYYSPN

SEQ ID NO: 117 - protein, 16 aa, Artificial Sequence
  Description of Artificial Sequence: Synthetic peptide
  Mass spectrometry peptide #117
NNEGTYYSPNYNPQSR

SEQ ID NO: 118 - protein, 6 aa, Artificial Sequence
  Description of Artificial Sequence: Synthetic peptide
  Mass spectrometry peptide #118
PLIICK

SEQ ID NO: 119 - protein, 14 aa, Artificial Sequence
  Description of Artificial Sequence: Synthetic peptide
  Mass spectrometry peptide #119
PTKDIFTGLIGPMK

SEQ ID NO: 120 - protein, 6 aa, Artificial Sequence
  Description of Artificial Sequence: Synthetic peptide
  Mass spectrometry peptide #120
PVCLAK

SEQ ID NO: 121 - protein, 12 aa, Artificial Sequence
  Description of Artificial Sequence: Synthetic peptide
  Mass spectrometry peptide #121
PVWLGFLGPIIK

SEQ ID NO: 122 - protein, 9 aa, Artificial Sequence
  Description of Artificial Sequence: Synthetic peptide
  Mass spectrometry peptide #122
QKYTVNQCR

SEQ ID NO: 123 - protein, 12 aa, Artificial Sequence
  Description of Artificial Sequence: Synthetic peptide
  Mass spectrometry peptide #123
QSEDSTFYLGER

SEQ ID NO: 124 - protein, 14 aa, Artificial Sequence
  Description of Artificial Sequence: Synthetic peptide
  Mass spectrometry peptide #124
QVDKEDEDFQESNK

SEQ ID NO: 125 - protein, 8 aa, Artificial Sequence
  Description of Artificial Sequence: Synthetic peptide
  Mass spectrometry peptide #125
QYTDSTFR

SEQ ID NO: 126 - protein, 13 aa, Artificial Sequence
  Description of Artificial Sequence: Synthetic peptide
  Mass spectrometry peptide #126
QYTDSTFRVPVER

SEQ ID NO: 127 - protein, 12 aa, Artificial Sequence
  Description of Artificial Sequence: Synthetic peptide
  Mass spectrometry peptide #127
REYTDASFTNRK

SEQ ID NO: 128 - protein, 13 aa, Artificial Sequence
  Description of Artificial Sequence: Synthetic peptide
  Mass spectrometry peptide #128
RQSEDSTFYLGER

SEQ ID NO: 129 - protein, 21 aa, Artificial Sequence
  Description of Artificial Sequence: Synthetic peptide
  Mass spectrometry peptide #129
SAGNEADVHGIYFSGNTYLWR

SEQ ID NO: 130 - protein, 5 aa, Artificial Sequence
  Description of Artificial Sequence: Synthetic peptide
  Mass spectrometry peptide #130
SFQYK

SEQ ID NO: 131 - protein, 23 aa, Artificial Sequence
  Description of Artificial Sequence: Synthetic peptide
  Mass spectrometry peptide #131
SGAGTEDSACIPWAYYSTVDQVK

SEQ ID NO: 132 - protein, 11 aa, Artificial Sequence
  Description of Artificial Sequence: Synthetic peptide
  Mass spectrometry peptide #132
SGLIGPLIVCR

SEQ ID NO: 133 - protein, 8 aa, Artificial Sequence
  Description of Artificial Sequence: Synthetic peptide
  Mass spectrometry peptide #133
SHGITYYK

SEQ ID NO: 134 - protein, 8 aa, Artificial Sequence
  Description of Artificial Sequence: Synthetic peptide
  Mass spectrometry peptide #134
SHGITYYK

SEQ ID NO: 135 - protein, 8 aa, Artificial Sequence
  Description of Artificial Sequence: Synthetic peptide
  Mass spectrometry peptide #135
SIEPIGVR

SEQ ID NO: 136 - protein, 9 aa, Artificial Sequence
  Description of Artificial Sequence: Synthetic peptide
  Mass spectrometry peptide #136
SPNYNPQSR

SEQ ID NO: 137 - protein, 20 aa, Artificial Sequence
  Description of Artificial Sequence: Synthetic peptide
  Mass spectrometry peptide #137
SSDVFDIFPGTYQTLEMFPR

SEQ ID NO: 138 - protein, 17 aa, Artificial Sequence
  Description of Artificial Sequence: Synthetic peptide
  Mass spectrometry peptide #138
SVDTEHSNIYLQNGPDR

SEQ ID NO: 139 - protein, 23 aa, Artificial Sequence
  Description of Artificial Sequence: Synthetic peptide
  Mass spectrometry peptide #139
SVPPSASHVAPTETFTYEWTVPK

SEQ ID NO: 140 - protein, 16 aa, Artificial Sequence
  Description of Artificial Sequence: Synthetic peptide
  Mass spectrometry peptide #140
SVVDENFSWYLEDNIK

SEQ ID NO: 141 - protein, 8 aa, Artificial Sequence
  Description of Artificial Sequence: Synthetic peptide
  Mass spectrometry peptide #141
TDASFTNR

SEQ ID NO: 142 - protein, 10 aa, Artificial Sequence
  Description of Artificial Sequence: Synthetic peptide
  Mass spectrometry peptide #142
TPGIWLLHCH

SEQ ID NO: 143 - protein, 19 aa, Artificial Sequence
  Description of Artificial Sequence: Synthetic peptide
  Mass spectrometry peptide #143
TTAPDQVDKEDEDFQESNK

SEQ ID NO: 144 - protein, 11 aa, Artificial Sequence
  Description of Artificial Sequence: Synthetic peptide
  Mass spectrometry peptide #144
TTIEKPVWLGF

SEQ ID NO: 145 - protein, 17 aa, Artificial Sequence
  Description of Artificial Sequence: Synthetic peptide
  Mass spectrometry peptide #145
TTIEKPVWLGFLGPIIK

SEQ ID NO: 146 - protein, 9 aa, Artificial Sequence
  Description of Artificial Sequence: Synthetic peptide
  Mass spectrometry peptide #146
TVLQNEDTK

SEQ ID NO: 147 - protein, 8 aa, Artificial Sequence
  Description of Artificial Sequence: Synthetic peptide
  Mass spectrometry peptide #147
TYCSEPEK

SEQ ID NO: 148 - protein, 21 aa, Artificial Sequence
  Description of Artificial Sequence: Synthetic peptide
  Mass spectrometry peptide #148
TYCSEPEKVDKDNEDFQESNR

SEQ ID NO: 149 - protein, 5 aa, Artificial Sequence
  Description of Artificial Sequence: Synthetic peptide
  Mass spectrometry peptide #149
TYLWR

SEQ ID NO: 150 - protein, 8 aa, Artificial Sequence
  Description of Artificial Sequence: Synthetic peptide
  Mass spectrometry peptide #150
TYSDHPEK

SEQ ID NO: 151 - protein, 17 aa, Artificial Sequence
  Description of Artificial Sequence: Synthetic peptide
  Mass spectrometry peptide #151
TYYIAAVEVEWDYSPQR

SEQ ID NO: 152 - protein, 12 aa, Artificial Sequence
  Description of Artificial Sequence: Synthetic peptide
  Mass spectrometry peptide #152
TYYSPNYNPQSR

SEQ ID NO: 153 - protein, 12 aa, Artificial Sequence
  Description of Artificial Sequence: Synthetic peptide
  Mass spectrometry peptide #153
VDKDNEDFQESN

SEQ ID NO: 154 - protein, 13 aa, Artificial Sequence
  Description of Artificial Sequence: Synthetic peptide
  Mass spectrometry peptide #154
VDKDNEDFQESNR

SEQ ID NO: 155 - protein, 12 aa, Artificial Sequence
  Description of Artificial Sequence: Synthetic peptide
  Mass spectrometry peptide #155
VNKDDEEFIESN

SEQ ID NO: 156 - protein, 13 aa, Artificial Sequence
  Description of Artificial Sequence: Synthetic peptide
  Mass spectrometry peptide #156
VNKDDEEFIESNK

SEQ ID NO: 157 - protein, 6 aa, Artificial Sequence
  Description of Artificial Sequence: Synthetic peptide
  Mass spectrometry peptide #157
VPVERK

SEQ ID NO: 158 - protein, 6 aa, Artificial Sequence
  Description of Artificial Sequence: Synthetic peptide
  Mass spectrometry peptide #158
VTFHNK

SEQ ID NO: 159 - protein, 19 aa, Artificial Sequence
  Description of Artificial Sequence: Synthetic peptide
  Mass spectrometry peptide #159
VTFHNKGAYPLSIEPIGVR

SEQ ID NO: 160 - protein, 12 aa, Artificial Sequence
  Description of Artificial Sequence: Synthetic peptide
  Mass spectrometry peptide #160
VVYRQYTDSTFR

SEQ ID NO: 161 - protein, 9 aa, Artificial Sequence
  Description of Artificial Sequence: Synthetic peptide
  Mass spectrometry peptide #161
VYPGEQYTY

SEQ ID NO: 162 - protein, 29 aa, Artificial Sequence
  Description of Artificial Sequence: Synthetic peptide
  Mass spectrometry peptide #162
VYPGEQYTYMLLATEEQSPGEGDGNCVTR

SEQ ID NO: 163 - protein, 6 aa, Artificial Sequence
  Description of Artificial Sequence: Synthetic peptide
  Mass spectrometry peptide #163
VYVHLK

SEQ ID NO: 164 - protein, 10 aa, Artificial Sequence
  Description of Artificial Sequence: Synthetic peptide
  Mass spectrometry peptide #164
YSPNYNPQSR

SEQ ID NO: 165 - protein, 8 aa, Artificial Sequence
  Description of Artificial Sequence: Synthetic peptide
  Mass spectrometry peptide #165
YSTVDQVK

SEQ ID NO: 166 - protein, 7 aa, Artificial Sequence
  Description of Artificial Sequence: Synthetic peptide
  Mass spectrometry peptide #166
YTVNQCR

SEQ ID NO: 167 - protein, 9 aa, Artificial Sequence
  Description of Artificial Sequence: Synthetic peptide
  Mass spectrometry peptide #167
IDVHLVPDR

SEQ ID NO: 168 - protein, 8 aa, Artificial Sequence
  Description of Artificial Sequence: Synthetic peptide
  Mass spectrometry peptide #168
NLPNGDFR

SEQ ID NO: 169 - protein, 15 aa, Artificial Sequence
  Description of Artificial Sequence: Synthetic peptide
  Mass spectrometry peptide #169
QDACQGDSGGVFAVR

SEQ ID NO: 170 - protein, 9 aa, Artificial Sequence
  Description of Artificial Sequence: Synthetic peptide
  Mass spectrometry peptide #170
VLNYVDWIK

SEQ ID NO: 171 - protein, 12 aa, Artificial Sequence
  Description of Artificial Sequence: Synthetic peptide
  Mass spectrometry peptide #171
AYSLFSYNTQGR

SEQ ID NO: 172 - protein, 11 aa, Artificial Sequence
  Description of Artificial Sequence: Synthetic peptide
  Mass spectrometry peptide #172
GYVIIKPLVWV

SEQ ID NO: 173 - protein, 13 aa, Artificial Sequence
  Description of Artificial Sequence: Synthetic peptide
  Mass spectrometry peptide #173
IVLGQEQDSYGGK

SEQ ID NO: 174 - protein, 6 aa, Artificial Sequence
  Description of Artificial Sequence: Synthetic peptide
  Mass spectrometry peptide #174
VFVFPR

SEQ ID NO: 175 - protein, 10 aa, Artificial Sequence
  Description of Artificial Sequence: Synthetic peptide
  Mass spectrometry peptide #175
VGEYSLYIGR

SEQ ID NO: 176 - protein, 15 aa, Artificial Sequence
  Description of Artificial Sequence: Synthetic peptide
  Mass spectrometry peptide #176
ADSPMDDFFQCVNGK

SEQ ID NO: 177 - protein, 13 aa, Artificial Sequence
  Description of Artificial Sequence: Synthetic peptide
  Mass spectrometry peptide #177
AQLGDLPWQVAIK

SEQ ID NO: 178 - protein, 19 aa, Artificial Sequence
  Description of Artificial Sequence: Synthetic peptide
  Mass spectrometry peptide #178
ASVAQEETEILTADMDAER

SEQ ID NO: 179 - protein, 6 aa, Artificial Sequence
  Description of Artificial Sequence: Synthetic peptide
  Mass spectrometry peptide # 179
DCELPR

SEQ ID NO: 180 - protein, 14 aa, Artificial Sequence
  Description of Artificial Sequence: Synthetic peptide
  Mass spectrometry peptide #180
DNERVFSLQWGEVK

SEQ ID NO: 181 - protein, 19 aa, Artificial Sequence
  Description of Artificial Sequence: Synthetic peptide
  Mass spectrometry peptide #181
EANVACLDLGFQQGADTQR

SEQ ID NO: 182 - protein, 7 aa, Artificial Sequence
  Description of Artificial Sequence: Synthetic peptide
  Mass spectrometry peptide #182
FISQYNV

SEQ ID NO: 183 - protein, 6 aa, Artificial Sequence
  Description of Artificial Sequence: Synthetic peptide
  Mass spectrometry peptide #183
FSVSLK

SEQ ID NO: 184 - protein, 5 aa, Artificial Sequence
  Description of Artificial Sequence: Synthetic peptide
  Mass spectrometry peptide #184
FYGNR

SEQ ID NO: 185 - protein, 5 aa, Artificial Sequence
  Description of Artificial Sequence: Synthetic peptide
  Mass spectrometry peptide #185
GFHCK

SEQ ID NO: 186 - protein, 13 aa, Artificial Sequence
  Description of Artificial Sequence: Synthetic peptide
  Mass spectrometry peptide #186
GLETSLAECTFTK

SEQ ID NO: 187 - protein, 13 aa, Artificial Sequence
  Description of Artificial Sequence: Synthetic peptide
  Mass spectrometry peptide #187
HGNTDSEGIVEVK

SEQ ID NO: 188 - protein, 6 aa, Artificial Sequence
  Description of Artificial Sequence: Synthetic peptide
  Mass spectrometry peptide #188
IVGGKR

SEQ ID NO: 189 - protein, 8 aa, Artificial Sequence
  Description of Artificial Sequence: Synthetic peptide
  Mass spectrometry peptide No. 189
IVIEYVDR

SEQ ID NO: 190 - protein, 13 aa, Artificial Sequence
  Description of Artificial Sequence: Synthetic peptide
  Mass spectrometry peptide #190
KVTYTSQEDLVEK

SEQ ID NO: 191 - protein, 7 aa, Artificial Sequence
  Description of Artificial Sequence: Synthetic peptide
  Mass spectrometry peptide #191
LISNCSK

SEQ ID NO: 192 - protein, 7 aa, Artificial Sequence
  Description of Artificial Sequence: Synthetic peptide
  Mass spectrometry peptide #192
LPYQCPK

SEQ ID NO: 193 - protein, 6 aa, Artificial Sequence
  Description of Artificial Sequence: Synthetic peptide
  Mass spectrometry peptide #193
LVDQDK

SEQ ID NO: 194 - protein, 10 aa, Artificial Sequence
  Description of Artificial Sequence: Synthetic peptide
  Mass spectrometry peptide #194
NGTAVCATNR

SEQ ID NO: 195 - protein, 8 aa, Artificial Sequence
  Description of Artificial Sequence: Synthetic peptide
  Mass spectrometry peptide #195
PFISQYNV

SEQ ID NO: 196 - protein, 14 aa, Artificial Sequence
  Description of Artificial Sequence: Synthetic peptide
  Mass spectrometry peptide #196
RAQLGDLPWQVAIK

SEQ ID NO: 197 - protein, 9 aa, Artificial Sequence
  Description of Artificial Sequence: Synthetic peptide
  Mass spectrometry peptide #197
SFPTYCQQK

SEQ ID NO: 198 - protein, 10 aa, Artificial Sequence
  Description of Artificial Sequence: Synthetic peptide
  Mass spectrometry peptide #198
SLECLHPGTK

SEQ ID NO: 199 - protein, 5 aa, Artificial Sequence
  Description of Artificial Sequence: Synthetic peptide
  Mass spectrometry peptide #199
SLLPK

SEQ ID NO: 200 - protein, 6 aa, Artificial Sequence
  Description of Artificial Sequence: Synthetic peptide
  Mass spectrometry peptide #200
SSWSMR

SEQ ID NO: 201 - protein, 6 aa, Artificial Sequence
  Description of Artificial Sequence: Synthetic peptide
  Mass spectrometry peptide #201
TMFICK

SEQ ID NO: 202 - protein, 16 aa, Artificial Sequence
  Description of Artificial Sequence: Synthetic peptide
  Mass spectrometry peptide #202
TMGYQDFADVVCYTQK

SEQ ID NO: 203 - protein, 14 aa, Artificial Sequence
  Description of Artificial Sequence: Synthetic peptide
  Mass spectrometry peptide #203
VANYFDWISYHVGR

SEQ ID NO: 204 - protein, 22 aa, Artificial Sequence
  Description of Artificial Sequence: Synthetic peptide
  Mass spectrometry peptide #204
VANYFDWISYHVGRPFISQYNV

SEQ ID NO: 205 - protein, 8 aa, Artificial Sequence
  Description of Artificial Sequence: Synthetic peptide
  Mass spectrometry peptide #205
VFCQPWQR

SEQ ID NO: 206 - protein, 10 aa, Artificial Sequence
  Description of Artificial Sequence: Synthetic peptide
  Mass spectrometry peptide #206
VFSLQWGEVK

SEQ ID NO: 207 - protein, 12 aa, Artificial Sequence
  Description of Artificial Sequence: Synthetic peptide
  Mass spectrometry peptide #207
VTYTSQEDLVEK

SEQ ID NO: 208 - protein, 6 aa, Artificial Sequence
  Description of Artificial Sequence: Synthetic peptide
  Mass spectrometry peptide #208
YISQMK

SEQ ID NO: 209 - protein, 6 aa, Artificial Sequence
  Description of Artificial Sequence: Synthetic peptide
  Mass spectrometry peptide #209
YISQMK

SEQ ID NO: 210 - protein, 16 aa, Artificial Sequence
  Description of Artificial Sequence: Synthetic peptide
  Mass spectrometry peptide #210
YQIWTTVVDWIHPDLK

SEQ ID NO: 211 - protein, 17 aa, Artificial Sequence
  Description of Artificial Sequence: Synthetic peptide
  Mass spectrometry peptide #211
YQIWTTVVDWIHPDLKR

SEQ ID NO: 212 - protein, 12 aa, Artificial Sequence
  Description of Artificial Sequence: Synthetic peptide
  Mass spectrometry peptide #212
LQSLFDSPDFSK

SEQ ID NO: 213 - protein, 9 aa, Artificial Sequence
  Description of Artificial Sequence: Synthetic peptide
  Mass spectrometry peptide #213
LSYEGEVTK

SEQ ID NO: 214 - protein, 8 aa, Artificial Sequence
  Description of Artificial Sequence: Synthetic peptide
  Mass spectrometry peptide #214
APYPNYDR

SEQ ID NO: 215 - protein, 12 aa, Artificial Sequence
  Description of Artificial Sequence: Synthetic peptide
  Mass spectrometry peptide #215
ASEGGFTATGQR

SEQ ID NO: 216 - protein, 8 aa, Artificial Sequence
  Description of Artificial Sequence: Synthetic peptide
  Mass spectrometry peptide No. 216
AWEDTLDK

SEQ ID NO: 217 - protein, 6 aa, Artificial Sequence
  Description of Artificial Sequence: Synthetic peptide
  seq id no. 217
DECFAR

SEQ ID NO: 218 - protein, 9 aa, Artificial Sequence
  Description of Artificial Sequence: Synthetic peptide
  Mass spectrometry peptide #218
DILTIDIGR

SEQ ID NO: 219 - protein, 10 aa, Artificial Sequence
  Description of Artificial Sequence: Synthetic peptide
  Mass spectrometry peptide #219
EGGFTATGQR

SEQ ID NO: 220 - protein, 18 aa, Artificial Sequence
  Description of Artificial Sequence: Synthetic peptide
  Mass spectrometry peptide #220
EHFQEVGYAAPPSPPLSR

SEQ ID NO: 221 - protein, 10 aa, Artificial Sequence
  Description of Artificial Sequence: Synthetic peptide
  Mass spectrometry peptide #221
ELLALIQLER

SEQ ID NO: 222 - protein, 12 aa, Artificial Sequence
  Description of Artificial Sequence: Synthetic peptide
  Mass spectrometry peptide #222
ELPSLQHPNEQK

SEQ ID NO: 223 - protein, 15 aa, Artificial Sequence
  Description of Artificial Sequence: Synthetic peptide
  Mass spectrometry peptide #223
EVGPPLPQEAVPLQK

SEQ ID NO: 224 - protein, 5 aa, Artificial Sequence
  Description of Artificial Sequence: Synthetic peptide
  Mass spectrometry peptide #224
EYAVK

SEQ ID NO: 225 - protein, 16 aa, Artificial Sequence
  Description of Artificial Sequence: Synthetic peptide
  Mass spectrometry peptide #225
FSCFQEEAPQPHYQLR

SEQ ID NO: 226 - protein, 7 aa, Artificial Sequence
  Description of Artificial Sequence: Synthetic peptide
  Mass spectrometry peptide #226
HPPSPTR

SEQ ID NO: 227 - protein, 9 aa, Artificial Sequence
  Description of Artificial Sequence: Synthetic peptide
  Mass spectrometry peptide #227
LPAQLPAEK

SEQ ID NO: 228 - protein, 11 aa, Artificial Sequence
  Description of Artificial Sequence: Synthetic peptide
  Mass spectrometry peptide #228
LTFINDLCGPR

SEQ ID NO: 229 - protein, 9 aa, Artificial Sequence
  Description of Artificial Sequence: Synthetic peptide
  Mass spectrometry peptide #229
LVWEEAMSR

SEQ ID NO: 230 - protein, 9 aa, Artificial Sequence
  Description of Artificial Sequence: Synthetic peptide
  Mass spectrometry peptide #230
NFLEIGYSR

SEQ ID NO: 231 - protein, 6 aa, Artificial Sequence
  Description of Artificial Sequence: Synthetic peptide
  Mass spectrometry peptide #231
NICHLR

SEQ ID NO: 232 - protein, 10 aa, Artificial Sequence
  Description of Artificial Sequence: Synthetic peptide
  Mass spectrometry peptide #232
NLPATDPLQR

SEQ ID NO: 233 - protein, 13 aa, Artificial Sequence
  Description of Artificial Sequence: Synthetic peptide
  Mass spectrometry peptide #233
NVALVSGDTENAK

SEQ ID NO: 234 - protein, 10 aa, Artificial Sequence
  Description of Artificial Sequence: Synthetic peptide
  Mass spectrometry peptide #234
PHSQPWLGER

SEQ ID NO: 235 - protein, 14 aa, Artificial Sequence
  Description of Artificial Sequence: Synthetic peptide
  Mass spectrometry peptide #235
QGETLNFLEIGYSR

SEQ ID NO: 236 - protein, 19 aa, Artificial Sequence
  Description of Artificial Sequence: Synthetic peptide
  Mass spectrometry peptide #236
QLRPEHFQEVGYAAPPSPP

SEQ ID NO: 237 - protein, 22 aa, Artificial Sequence
  Description of Artificial Sequence: Synthetic peptide
  Mass spectrometry peptide #237
QLRPEHFQEVGYAAPPSPPLSR

SEQ ID NO: 238 - protein, 11 aa, Artificial Sequence
  Description of Artificial Sequence: Synthetic peptide
  Mass spectrometry peptide #238
SEGGFTATGQR

SEQ ID NO: 239 - protein, 14 aa, Artificial Sequence
  Description of Artificial Sequence: Synthetic peptide
  Mass spectrometry peptide #239
VTPNLMGHLCGNQR

SEQ ID NO: 240 - protein, 18 aa, Artificial Sequence
  Description of Artificial Sequence: Synthetic peptide
  Mass spectrometry peptide #240
FNAVLTNPQGDYDTSTGK

SEQ ID NO: 241 - protein, 9 aa, Artificial Sequence
  Description of Artificial Sequence: Synthetic peptide
  Mass spectrometry peptide #241
FQSVFTVTR

SEQ ID NO: 242 - protein, 11 aa, Artificial Sequence
  Description of Artificial Sequence: Synthetic peptide
  Mass spectrometry peptide #242
GIPGEPGEEGR

SEQ ID NO: 243 - protein, 11 aa, Artificial Sequence
  Description of Artificial Sequence: Synthetic peptide
  Mass spectrometry peptide #243
GMPGLPGAPGK

SEQ ID NO: 244 - protein, 27 aa, Artificial Sequence
  Description of Artificial Sequence: Synthetic peptide
  Mass spectrometry peptide #244
NGPMGPPGMPGVPGPMGIPGEPGEEGR

SEQ ID NO: 245 - protein, 11 aa, Artificial Sequence
  Description of Artificial Sequence: Synthetic peptide
  Mass spectrometry peptide #245
PQGDYDTSTGK

SEQ ID NO: 246 - protein, 11 aa, Artificial Sequence
  Description of Artificial Sequence: Synthetic peptide
  Mass spectrometry peptide #246
QKFQSVFTVTR

SEQ ID NO: 247 - protein, 13 aa, Artificial Sequence
  Description of Artificial Sequence: Synthetic peptide
  Mass spectrometry peptide #247
QTHQPPAPNSLIR

SEQ ID NO: 248 - protein, 12 aa, Artificial Sequence
  Description of Artificial Sequence: Synthetic peptide
  Mass spectrometry peptide #248
TNQVNSGGVLLR

SEQ ID NO: 249 - protein, 9 aa, Artificial Sequence
  Description of Artificial Sequence: Synthetic peptide
  Mass spectrometry peptide #249
ANSFLEEMK

SEQ ID NO: 250 - protein, 11 aa, Artificial Sequence
  Description of Artificial Sequence: Synthetic peptide
  Mass spectrometry peptide #250
DWAESTLMTQK

SEQ ID NO: 251 - protein, 9 aa, Artificial Sequence
  Description of Artificial Sequence: Synthetic peptide
  Mass spectrometry peptide #251
EQANNILAR

SEQ ID NO: 252 - protein, 11 aa, Artificial Sequence
  Description of Artificial Sequence: Synthetic peptide
  Mass spectrometry peptide #252
ETYDFDIAVLR

SEQ ID NO: 253 - protein, 8 aa, Artificial Sequence
  Description of Artificial Sequence: Synthetic peptide
  Mass spectrometry peptide #253
EVFEDSDK

SEQ ID NO: 254 - protein, 9 aa, Artificial Sequence
  Description of Artificial Sequence: Synthetic peptide
  Mass spectrometry peptide #254
GYTLADNGK

SEQ ID NO: 255 - protein, 8 aa, Artificial Sequence
  Description of Artificial Sequence: Synthetic peptide
  Mass spectrometry peptide #255
IVGGQECK

SEQ ID NO: 256 - protein, 8 aa, Artificial Sequence
  Description of Artificial Sequence: Synthetic peptide
  Mass spectrometry peptide #256
LKTPITFR

SEQ ID NO: 257 - protein, 9 aa, Artificial Sequence
  Description of Artificial Sequence: Synthetic peptide
  Mass spectrometry peptide #257
MLEVPYVDR

SEQ ID NO: 258 - protein, 11 aa, Artificial Sequence
  Description of Artificial Sequence: Synthetic peptide
  Mass spectrometry peptide # 258
MNVAPACLPER

SEQ ID NO: 259 - protein, 22 aa, Artificial Sequence
  Description of Artificial Sequence: Synthetic peptide
  Mass spectrometry peptide #259
MNVAPACLPERDWAESTLMTQK

SEQ ID NO: 260 - protein, 19 aa, Artificial Sequence
  Description of Artificial Sequence: Synthetic peptide
  Mass spectrometry peptide #260
NTEQEEGGEAVHEVEVVIK

SEQ ID NO: 261 - protein, 16 aa, Artificial Sequence
  Description of Artificial Sequence: Synthetic peptide
  Mass spectrometry peptide #261
QEDACQGDSGGPHVTR

SEQ ID NO: 262 - protein, 9 aa, Artificial Sequence
  Description of Artificial Sequence: Synthetic peptide
  Mass spectrometry peptide #262
TGIVSGFGR

SEQ ID NO: 263 - protein, 7 aa, Artificial Sequence
  Description of Artificial Sequence: Synthetic peptide
  Mass spectrometry peptide #263
TNEFWNK

SEQ ID NO: 264 - protein, 6 aa, Artificial Sequence
  Description of Artificial Sequence: Synthetic peptide
  Mass spectrometry peptide #264
TPITFR

SEQ ID NO: 265 - protein, 6 aa, Artificial Sequence
  Description of Artificial Sequence: Synthetic peptide
  Mass spectrometry peptide #265
TRGLPK

SEQ ID NO: 266 - protein, 6 aa, Artificial Sequence
  Description of Artificial Sequence: Synthetic peptide
  Mass spectrometry peptide #266
VTAFLK

SEQ ID NO: 267 - protein, 17 aa, Artificial Sequence
  Description of Artificial Sequence: Synthetic peptide
  Mass spectrometry peptide #267
YKDGDQCETSPCQNQGK

SEQ ID NO: 268 - protein, 15 aa, Artificial Sequence
  Description of Artificial Sequence: Synthetic peptide
  Mass spectrometry peptide #268
IFYNQQNHYDGSTGK

SEQ ID NO: 269 - protein, 11 aa, Artificial Sequence
  Description of Artificial Sequence: Synthetic peptide
  Mass spectrometry peptide #269
ALDFAVGEYNK

SEQ ID NO: 270 - protein, 6 aa, Artificial Sequence
  Description of Artificial Sequence: Synthetic peptide
  Mass spectrometry peptide #270
ALQVVR

SEQ ID NO: 271 - protein, 9 aa, Artificial Sequence
  Description of Artificial Sequence: Synthetic peptide
  Mass spectrometry peptide #271
ASNDMYHSR

SEQ ID NO: 272 - protein, 15 aa, Artificial Sequence
  Description of Artificial Sequence: Synthetic peptide
  Mass spectrometry peptide #272
ASNDMYHSRALQVVR

SEQ ID NO: 273 - protein, 16 aa, Artificial Sequence
  Description of Artificial Sequence: Synthetic peptide
  Mass spectrometry peptide #273
LVGGPMDASVEEEGVR

SEQ ID NO: 274 - protein, 17 aa, Artificial Sequence
  Description of Artificial Sequence: Synthetic peptide
  Mass spectrometry peptide #274
LVGGPMDASVEEEGVRR

SEQ ID NO: 275 - protein, 5 aa, Artificial Sequence
  Description of Artificial Sequence: Synthetic peptide
  Mass spectrometry peptide #275
MTLSK

SEQ ID NO: 276 - protein, 17 aa, Artificial Sequence
  Description of Artificial Sequence: Synthetic peptide
  Mass spectrometry peptide #276
NKASNDMYHSRALQVVR

SEQ ID NO: 277 - protein, 8 aa, Artificial Sequence
  Description of Artificial Sequence: Synthetic peptide
  Mass spectrometry peptide #277
SRALQVVR

SEQ ID NO: 278 - protein, 17 aa, Artificial Sequence
  Description of Artificial Sequence: Synthetic peptide
  Mass spectrometry peptide #278
TQPNLDNCPFHDQPHLK

SEQ ID NO: 279 - protein, 22 aa, Artificial Sequence
  Description of Artificial Sequence: Synthetic peptide
  Mass spectrometry peptide #279
ALALPPLGLAPLLNLWAKPQGR

SEQ ID NO: 280 - protein, 9 aa, Artificial Sequence
  Description of Artificial Sequence: Synthetic peptide
  Mass spectrometry peptide #280
DDWFMLGLR

SEQ ID NO: 281 - protein, 6 aa, Artificial Sequence
  Description of Artificial Sequence: Synthetic peptide
  Mass spectrometry peptide #281
DSWLDK

SEQ ID NO: 282 - protein, 8 aa, Artificial Sequence
  Description of Artificial Sequence: Synthetic peptide
  Mass spectrometry peptide #282
GSPAVLFK

SEQ ID NO: 283 - protein, 14 aa, Artificial Sequence
  Description of Artificial Sequence: Synthetic peptide
  Mass spectrometry peptide #283
HPIMRIALGGLLFP

SEQ ID NO: 284 - protein, 14 aa, Artificial Sequence
  Description of Artificial Sequence: Synthetic peptide
  Mass spectrometry peptide #284
IALGGLLFPASNLR

SEQ ID NO: 285 - protein, 12 aa, Artificial Sequence
  Description of Artificial Sequence: Synthetic peptide
  Mass spectrometry peptide #285
LPLVPALDGCLR

SEQ ID NO: 286 - protein, 10 aa, Artificial Sequence
  Description of Artificial Sequence: Synthetic peptide
  Mass spectrometry peptide #286
LVPALDGCLR

SEQ ID NO: 287 - protein, 13 aa, Artificial Sequence
  Description of Artificial Sequence: Synthetic peptide
  Mass spectrometry peptide #287
QAEISASAPTSLR

SEQ ID NO: 288 - protein, 9 aa, Artificial Sequence
  Description of Artificial Sequence: Synthetic peptide
  Mass spectrometry peptide #288
QVSGPLTSK

SEQ ID NO: 289 - protein, 23 aa, Artificial Sequence
  Description of Artificial Sequence: Synthetic peptide
  Mass spectrometry peptide #289
SCDVESNPGIFLPPGTQAEFNLR

SEQ ID NO: 290 - protein, 8 aa, Artificial Sequence
  Description of Artificial Sequence: Synthetic peptide
  Mass spectrometry peptide #290
TSSSFEVR

SEQ ID NO: 291 - protein, 8 aa, Artificial Sequence
  Description of Artificial Sequence: Synthetic peptide
  Mass spectrometry peptide #291
VVLSQGSK

SEQ ID NO: 292 - protein, 24 aa, Artificial Sequence
  Description of Artificial Sequence: Synthetic peptide
  Mass spectrometry peptide #292
VVLSSGSGPGLDLPLVLGLPLQLK

SEQ ID NO: 293 - protein, 7 aa, Artificial Sequence
  Description of Artificial Sequence: Synthetic peptide
  Mass spectrometry peptide #293
WHQVEVK

SEQ ID NO: 294 - protein, 11 aa, Artificial Sequence
  Description of Artificial Sequence: Synthetic peptide
  Mass spectrometry peptide #294
AVEPQLQEEER

SEQ ID NO: 295 - protein, 14 aa, Artificial Sequence
  Description of Artificial Sequence: Synthetic peptide
  Mass spectrometry peptide #295
DPVASTSNLDMDFR

SEQ ID NO: 296 - protein, 5 aa, Artificial Sequence
  Description of Artificial Sequence: Synthetic peptide
  Mass spectrometry peptide #296
ERTWR

SEQ ID NO: 297 - protein, 15 aa, Artificial Sequence
  Description of Artificial Sequence: Synthetic peptide
  Mass spectrometry peptide #297
FLEQELETITIPDLR

SEQ ID NO: 298 - protein, 9 aa, Artificial Sequence
  Description of Artificial Sequence: Synthetic peptide
  Mass spectrometry peptide #298
GAFFPLTER

SEQ ID NO: 299 - protein, 16 aa, Artificial Sequence
  Description of Artificial Sequence: Synthetic peptide
  Mass spectrometry peptide #299
GAFFPLTERNWSLPNR

SEQ ID NO: 300 - protein, 6 aa, Artificial Sequence
  Description of Artificial Sequence: Synthetic peptide
  Mass spectrometry peptide #300
GGTFKK

SEQ ID NO: 301 - protein, 6 aa, Artificial Sequence
  Description of Artificial Sequence: Synthetic peptide
  Mass spectrometry peptide #301
TQLDLR

SEQ ID NO: 302 - protein, 10 aa, Artificial Sequence
  Description of Artificial Sequence: Synthetic peptide
  Mass spectrometry peptide #302
VPHDLDMLLR

SEQ ID NO: 303 - protein, 6 aa, Artificial Sequence
  Description of Artificial Sequence: Synthetic peptide
  Mass spectrometry peptide #303
DEYACR

SEQ ID NO: 304 - protein, 6 aa, Artificial Sequence
  Description of Artificial Sequence: Synthetic peptide
  Mass spectrometry peptide #304
IQVYSR

SEQ ID NO: 305 - protein, 10 aa, Artificial Sequence
  Description of Artificial Sequence: Synthetic peptide
  Mass spectrometry peptide #305
VNHVTLSQPK

SEQ ID NO: 306 - protein, 5 aa, Artificial Sequence
  Description of Artificial Sequence: Synthetic peptide
  Mass spectrometry peptide #306
WDRDM

SEQ ID NO: 307 - protein, 15 aa, Artificial Sequence
  Description of Artificial Sequence: Synthetic peptide
  Mass spectrometry peptide #307
YTEFTPTEKDEYACR

SEQ ID NO: 308-344 - skipped (37 entries)

SEQ ID NO: 345 - DNA, 243 bp, Homo sapiens
aagagtaagtatcacattcacaggatgaattaagaattgccaaggtgttcaatggataatggaaactgcagcatctcctgtcccccagccatgtcattctagctgtgaactggcacgaatgtcagcagcttttcattcagctgaatgctctttgagagattagggcagagaatggagtgttttaatgagttggaaattcatgctttgatgagatataagacctatgtgcatcttttctaatac

SEQ ID NO: 346 - DNA, 496 bp, Homo sapiens
gaaagttctagttgctcttcatgctcactaacatctgatattgttctccatttgattttagtcatgttagtagatgtaagatgttagttgcatttccctgatgagtactgatgttgagaacttttcatttgcttgttagccagtcacatattttcttttgtaaattgactgttccaatcttttacatgttcttaaatctcattatttttgttattcatatcaacagttaccaatagggcatttttatattttatcactagaaatcctttgtcacatatacatacctatattgcagatatttttcctcaaaggcatgtcttgcttattcattttcttttccttagtgatgtcttttgatgagcagaaatgtcttgatcatggtagttttatagtagtcttgaaatcaggtgatgtaagttatctttttcaagattgttttgactaatatagattctttgatttttccctaatgtggcaatcaacctttcctgccacc

SEQ ID NO: 347 - DNA, 487 bp, Homo sapiens
  modified_base (27)..(27): a, c, t, or g
  modified_base (292)..(292): a, c, t, or g
ggcagtctgcttaacaagatgctgggnaccacctctgaattagtgaatcagggagctgctgtgtgatcccctcctccaggctttcttggagtttgtctctgccacttcctcaagccctttaaaaaggctcctgtgctttagagagttaggatgttaatgaccgttcttttttcttgtactttattaagtactagtcttactagactagtgcttaagtggcagtgtctgcctttgccttctcccatctctttatgctcctttcttccaacttctcacgtttctgtttctcgcncggccaggtgtccagcatcccatgctgacttgcggttggccaatcccctcctcgtagatttcctccacattttcctcttcctccttttcctgtgggaagttctttttctgggctgtggctggtcttacgtagttcggtggaaaaagtattgaggtagtgttagatactgtgctgcacctccaggagtgtccaatc

SEQ ID NO: 348 - DNA, 369 bp, Homo sapiens
gtatgggaatcccttgagtttttcagtaaaattctatttgggtggctgtcaagtccagtctgcatagtcttgacatgtcttggataagatccttatactatcacttttcctgtcatggacactacaaattatattgaatcagaaatctaatttaatccattcttattcaattggaagcagtaattgctttcaatttaatgggattggattcagtattagaaattttgatgttccttcaaacttaatacagtatggttatatatatttccatttatttgtgtgtgttcctgtgccctcgcagagagatgatcttggagtattacttgtattcacttgagttatttgcaacggttggtaaagaggcatatt

SEQ ID NO: 349 - DNA, 558 bp, Homo sapiens
agatattttatccttttgaccttgatatgtttatagtaatttatttaacagtgtaagaaagagcttgaaatttgctttattagcatagttataatcttatttaatctgaaggatgtttcttaatttcatattcagaaacatatcctaatttatcatttaaacactgatataaaaatatgtctacttagtaacctattttttcttaaggtatttactatgcaggatctattacaactcattaaaatcaaccctacttccagtctctacaaatcactggtttcaggatctgataaagaaaatcaaaaaggtaggagccgtcatctttgtagagaacagcaacagttttctatgtactttttcctctaaaagaatgaagcaaatttttagggagaagagtaaaatttaatttgatttaaattatttgatttctgcaggtcatttcccctatatttccatttttgtaataacttttagatattcaattaaaaaaactgtagacagtatcaaaggccctacagacaggctgaaagatactgtttgtaaaaatacccactgttg

SEQ ID NO: 350 - DNA, 498 bp, Homo sapiens
  modified_base (71)..(71): a, c, t, or g
  modified_base (107)..(107): a, c, t, or g
  modified_base (451)..(451): a, c, t, or g
caaaatgcagcctgcaacttcctggagaacaactcagtgtcacattaaagtttattatgtatttaatgatncactgtttaattgacagttttgcatagtttgtctanctttagagaattaagagcctctcaactgagcagtaaaggtaaggagagctcaatctgcacagagccagtttttagtgtttgatggaaataagatcatcatgcccacttgagacttcagattattctttagcttagtggttgtatgagttacatcttattaaagtcgaaattaatgtagttttctgccttgataacatttcatatgtggtattagttttaaagggtcattaggaaaatgcacatattccatgaattttaagacccatagaaaagttgaagaatgcttaattttcttatccagtaatgtaaacacagagacagaacattgagatgtgcctagttcngtatttacagtttggtctggctgtttgagttctagcgcatttaatgt

SEQ ID NO: 351 - DNA, 450 bp, Homo sapiens
actattctcatcaacaccaggacttcgcctcctttttaagtggatttttcacctgtttttatcacagaagagctcttgtgagctggatgatagagagagctcagccttgctgagaggcagatgcaggtagttaccagctgcagtaataaactgcagccgggattcagcttcagccaagatagcacttctggtcttagagccacacaagtgatcagaatcacaaatgtgaaatccatgactgcccaaggcctgtcctcttctcctgctgaaggtctccagtagtgtatatcatgtttctgggctgctgtaacccatgctgttgtgtgtcacagtcctcagaactggagccctgaagacttaaaccggtcctaggaactgaagcattaaaccagaaagagctgtggggagagttgtcaacctagcccatccccaggatgtaatgactttgac

SEQ ID NO: 352 - DNA, 216 bp, Homo sapiens
  modified_base (121)..(122): a, c, t, or g
tcttcaccgtcctttcccagagaaagcgacagcacagcctatgacaaccgaagctaaaacggaaaaactgggccgggcgcaagaggaaaccagagccattcgactattgctagaggagagnnatagctgcaggaaaacatgcgcaaaatagcacaaacattgcgtcgtcaaggctgcaatgtctaatacaaaatagaaggttctcatagcaacaaa

SEQ ID NO: 353 - DNA, 428 bp, Homo sapiens
  modified_base (26)..(26): a, c, t, or g
  modified_base (35)..(35): a, c, t, or g
  modified_base (87)..(87): a, c, t, or g
  modified_base (103)..(103): a, c, t, or g
  modified_base (106)..(106): a, c, t, or g
  modified_base (108)..(108): a, c, t, or g
  modified_base (134)..(134): a, c, t, or g
  modified_base (140)..(140): a, c, t, or g
  modified_base (160)..(160): a, c, t, or g
  modified_base (200)..(200): a, c, t, or g
  modified_base (212)..(212): a, c, t, or g
  modified_base (238)..(238): a, c, t, or g
  modified_base (287)..(287): a, c, t, or g
  modified_base (300)..(300): a, c, t, or g
  modified_base (303)..(303): a, c, t, or g
  modified_base (342)..(342): a, c, t, or g
  modified_base (348)..(349): a, c, t, or g
  modified_base (352)..(352): a, c, t, or g
  modified_base (364)..(364): a, c, t, or g
atttacacaaggcaaataggagcttnggattattnggatggttattcaagtctagctaacttcaaagccagttaatttttttttttnaatacaggaagcgttnggngnaaataacagtctttccaaaaatgatngatatngcattctcaagaaggtcttngatagccaaatttctagcacctttagtagatgcctttctngcccctgaaacngtctcaagtatctgtattaaaccatngttaactttattcatgattctttagatagtaatgattaataatattagntaatatatatatngtnttagcatgtgccaggaatcataccaaatacttcatatgntacaanntgntgtgaagccagntggcacagagtaagctctcagtaaagtattagttgctttattgttattaaaaacgtgtctggct

SEQ ID NO: 354 - DNA, 492 bp, Homo sapiens
ttatagccactatcttttcacaatagtaaaacataaaagagtttaagctatgccaatttagagagtgcttaacaatgtgtaattactttattttcacttaatagatttttacctgtgccttgtttcagatctgaataggtttcctttgacctcaaaatcactaatgcagggaaaattaccatatcatgagagtatttttcctctttcttttttttgcctgatgcttagaataataaatactcccaaggcttaggagtagacatggcctgagagaaagcctattaaatgctattttctaatggttttaaaaaacaacttaaacaaatcatgctactcaaactgattttcttgctaaaacagaactagttttagagtatattttagacatctttttttctcaattactagttaaacttgaagttgggctctatctggatttgctttatagatctgcttcatttcatgccagatttgggcaaaccatgaacaacc

SEQ ID NO: 355 - DNA, 514 bp, Homo sapiens
  modified_base (117)..(117): a, c, t, or g
aatgcctgatttgctatttttgacatttcctcgtctcttaagaagtcagttaaatatgttttcatagtttatattcctgtttcatagattactgtgaaacatgtatttaaacctatngaattataaaatagtatttagattctagcgtgagttaaatagattagtcatatatcttttagatttgtggatttgacatgtaaattatgtgttgtgtataagtaagttagttactaaacatatggcatggttattgataaacttgttgctatttttttccaaatgctatcagtgtttgtggacttttaaaaattagtttgaattttggaatgttctgtgataaaatataatttcaactattttgtacatttaaatatgccatgttgtatatgtctgtatttaaaaatgttgtaaatatctgcattttaagaattatgaaagattttcctcaaaaatgacagaactctccatacttaattgtgacacattataagatatctgattttaagcttttgga

SEQ ID NO: 356 - DNA, 517 bp, Homo sapiens
gacaaaagccggtttctcagtgagggaggggcctccgaagcccgcgagaccttaaaatgaacctgccttccagctgcaagtgataccccacccccatctgggaggtaacccgacccctagggagtcacacagtccccagggaaacttcatgggggccctgtccgggagtgacccgcccccgcccccgcgaggggcttggtcatcctctgttagccgactacagaaagagtacccagagagcaaaacacacaccgaaagaatccagtctcccgcaaaagagattcaccccttagcattgccggaaggagaatctacaatttattttcggggaaatcgctggccccaagataatctgttaaattgtggactttatttccaagaaaaatacacctcatgcatctacacaaaactttcaaggagtccggagaacctatgaatcttaaggacctcaagagggaagcaaggaaattaacattattgaaataccagctgtgggccaggctttgttgttaggtat

SEQ ID NO: 357 - DNA, 312 bp, Homo sapiens
  modified_base (95)..(95): a, c, t, or g
  modified_base (265)..(265): a, c, t, or g
gcatgtcacggtgggtggctgtgtctgagcacctccagcagatgtcactctgagtgtgggtgttggtgacatgcattgcacgggcctgtctcccngtttgtgtaaacatactagagtatactgcggcgtgttttctgtctacccatgtcatggtgggggagatttatctccgtacatgtgggtgtcgccatgtgtgccctgtcactatctgtggctgggtgaacggctgtgtcattatgagtgtgccgagttatgccaccctgtntgctcagggcacatgcacacagacatttatctctgcactcacatttt

SEQ ID NO: 358 - DNA, 166 bp, Homo sapiens
gcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggg

SEQ ID NO: 359 - DNA, 468 bp, Homo sapiens
ggcttgatgtagactggcttgctttgatttttagtgaagggaatgtacgtaaaacaaaatagggcttggctggtcaaaggagacaagcaggatggatggatggatggatgaatagatagatggtgtttgcatgtaaattgcagagaaaacaaaaccaaagctgattggaaacaattaattgtgggtgtctgagggggaaggtcgcagctttgggcagctttgagaagcggtacaagagctctgtgcctgtgtgtccagccctggagccagccagtgcatttattttaagctcttagaagcaactccttggcccaggaatgcgtgacccctgagatgggtccacgcatctctctacacgtccttctctccgtgggatactggactcgtgcctctgcgcccattctcttctcacgcatatccatgagctttaatttcactttctgatcacggtacgtccataaagccagt

SEQ ID NO: 360 - DNA, 433 bp, Homo sapiens
ctaccaatgccgaggtgctgcgtgtgctgggcaagcccaagcctgaagagatgaatgtcaagatgctggactttgagacgttcttgcccatcctgcagcacatttcccgcaacaaggagcagggcacctatgaggacttcgtggagggcctgcgtgtctttgacaaggagagcaatggcacggtcatgggtgctgagcttcggcacgtccttgccaccctgggagagaagatgactgaggctgaagtggagcagctgttagctgggcaagaggatgccaatggctgcatcaattatgaagcctttgtcaagcacatcatgtcagggtgaagcagagtcttccaggtgcctggcccttggctttagccataccagggtgagttaaagagaggccccggctgggtgagctgagatggagtcctcgacttatcacc

SEQ ID NO: 361 - DNA, 532 bp, Homo sapiens
ttttgtttggacttcttttactcatgatcttgtgacatattatgtcttcatgcaaggggaaaatctcagcaatgattactctttgagatagaagaactgcaaaggtaataatacagccaaagataatctctcagcttttaaatgggtagagaaacactaaagcattcaatttattcaagaaaagtaagcccttgaagatatcttgaaatgaaagtataactgagttaaattatactggagaagtcttagacttgaaatactacttaccatatgtgcttgcctcagtaaaatgaaccccactgggtgggcagaggttcatttcaaatacatctttgatacttgttcaaaatatgttctttaaaaatataattttttagagagctgttcccaaattttctaacgagtggaccattatcactttaaagccctttatttataatacatttcctacgggctgtgttccaacaaccattttttttcagcagactatgaatattatagtattataggccaaactggcaaacttcagact

SEQ ID NO: 362 - DNA, 295 bp, Homo sapiens
tgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttg

SEQ ID NO: 363 - DNA, 501 bp, Homo sapiens
gtgctgtgctcagataataatagtttgtaagtaaaagtttttagttttcagtgttcaggttatagaatataactgaccataaaaattacctgcaggtattttctttttatgaacttgtttttaaattaccaagtaattactggtgtcattttgttttatgacagacacacgtatctaacaaacaaacaaacagtgaccttctccatgggtcaaggacttccttacaatttctcctgagttaacttttgtgaaaataatacctaaggttttctggcttattgaggaaatttcctaacaaacaaacaaacaaacaaacagaagagaagatcattaaccactgtatactttgtgtatataataggtcagtgtaaagaaatatgatttgaggtggtgcatgcaagtaactagggtttattctatataatgaatatttatagatctgtaacatttgtttcaaaatgctgtttcatttttataaagtaccagtgtttagctgctttt

SEQ ID NO: 364 - DNA, 531 bp, Homo sapiens
agtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtctgaaatctgcctcctgtgcctcccttttctcctctccccaatccatgcttcactcatgcaaactgctttattttccctacttgcagcgccaagtggccactggcatcgcagcttcctgtctgtttatatattaaagttgcttttattgcaccattttcaatttggagca

SEQ ID NO: 365 - DNA, 450 bp, Homo sapiens
tacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtc

SEQ ID NO: 366 - DNA, 511 bp, Homo sapiens
gcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtggga

SEQ ID NO: 367 - DNA, 71 bp, Homo sapiens
atgtaagagttcagactcacattctattaaaatttagccctaaaatgacaagccttcttaaagccttattt

SEQ ID NO: 368 - DNA, 459 bp, Homo sapiens
acattatgtggagccctatctttacaaaagtttcctactgtaaagtgcttttattttcagttttcatttgatagtactcaaccataattaaagttgcataagataattgctttacatttcacatacctatatttatctgagtgctgtctaaaactgttgtgctagccaaagtaatgctatgaaatcatttgcagaattaacccgtgagttaatgttaaatgcactgttattgccatgtgaagaggcatcgactttgataccaccatcatgttcagaccattttatacatttcagtggcctttttttttttaaggaaaaaaaagcgcaaaaccaagtacatagtgacgatggcttttatttggacaaatagcttttatattttcattaaaccatgcaaaaaatactacatctttctggcacataactgtctccttaaccactggaacagttcagccattt

SEQ ID NO: 369 - DNA, 135 bp, Homo sapiens
gaggcaaatggatctcgatatttcagatgggcttttgatgcactgttgccaaggaaggctttttctgattttttgacaaatgaatttttgcacactttcattggtgtctttcggcaacttacacacattgaaaat

SEQ ID NO: 370 - DNA, 433 bp, Homo sapiens; identical to SEQ ID NO: 360

SEQ ID NO: 371 - DNA, 498 bp, Homo sapiens
ctaccaatgccgaggtgctgcgtgtgctgggcaagcccaagcctgaagagatgaatgtcaagatgctggactttgagacgttcttgcccatcctgcagcacatttcccgcaacaaggagcagggcacctatgaggacttcgtggagggcctgcgtgtctttgacaaggagagcaatggcacggtcatgggtgctgagcttcggcacgtccttgccaccctgggagagaagatgactgaggctgaagtggagcagctgttagctgggcaagaggatgccaatggctgcatcaattatgaagcctttgtcaagcacatcatgtcagggtgaagcagagtcttccaggtgcctggcccttggctttagccataccagggtgagttaaagagaggccccggctgggtgagctgagatggagtcctcgacttatcaccacaccactgccccaaggaccttacaggccctccctgttaataaacagctctaacacggccaggct

SEQ ID NO: 372 - DNA, 545 bp, Homo sapiens
tggactttgagacgttcttgcccatcctgcagcacatttcccgcaacaaggagcagggcacctatgaggacttcgtggagggcctgcgtgtctttgacaaggagagcaatggcacggtcatgggtgctgagcttcggcacgtccttgccaccctgggagagaagatgactgaggctgaagtggagcagctgttagctgggcaagaggatgccaatggctgcatcaattatgaagcctttgtcaagcacatcatgtcagggtgaagcagagtcttccaggtgcctggcccttggctttagccataccagggtgagttaaagagaggccccggctgggtgagctgagatggagtcctcgacttatcaccacaccactgccccaaggaccttacaggccctccctgttaataaacagctctaacacggccaggctgggctctgggattctgagaccgcaagaccaacccttcctcctcctcagcctactcaacctgaagacaaggaagatgaaggccttgaagattatccacttccacttactagtaa

SEQ ID NO: 373 - DNA, 546 bp, Homo sapiens
gtccttgccaccctgggagagaagtcattgtttgaccggaccccgactggagagatgaagatcacctacatgactgaggctgaagtggagcagctgttagctgggcaagaggatggccagtgcggggatgtactgcgggccctgggccagaaccctaccgccaatggctgcatcaattatgaagcctttgtcaagcacatcatgaatgccgaggtgctgcgtgtgctgggcaagcccaagcctgaagagtcagggtgaagcagagtcttccaggtgcctgcccttggctttagccataccagggtgagttaaagagaggcccggctgggtgagctgagatggagtcctcgacttatcaccacaccactgccccaaggacttacaggccctccctgttaataaacagctctaacacggccaggctgggctctgtgaagcagagtcttccaggaccgcaagaccaacccttcctcctcctcagcctactcaacctgaagacaaggaagatgaaggccttgaagattatccacttccacttactagtaa

SEQ ID NO: 374 - DNA, 479 bp, Homo sapiens
  modified_base (441)..(441): a, c, t, or g
accaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttganaagattcttaatagctggctgtaaatactgcttggttt

SEQ ID NO: 375 - DNA, 493 bp, Homo sapiens
catcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggac

SEQ ID NO: 376 - DNA, 514 bp, Homo sapiens
tattgcatactacacagaagcttcaaattcctcatgttttaaatgagcactagagggaggtttatcacctttttggttaccagatgataatctatatttgttattactcagtgactctctaatttcacatcagcatgttcagcttgtgctagtcatcattctgatttgcttatctgtttttggtcaaaatatttatttttaaacatgttagaacttaccaaaatgaaagcaacagtgatgtattgaaatcttagttttgatattttaaagaaatgactttctagattaaaaaaatagttttgtagcattttaaacttagtgactatttagttcaattgttcatccattttttatttgcttttataattgcctccttgttttggtatattgtaaaataatttaaataatgtatttataaatatgtataattatgatgtaaaataccgtgtgtatatgtatgtatgtgtgtttctgtatatgcacacacataaacggctttgcctagccaaggatt

SEQ ID NO: 377 - DNA, 444 bp, Homo sapiens
gagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgatggtgc

SEQ ID NO: 378 - DNA, 297 bp, Homo sapiens
cctacctcattgttcttaatgcattgagaggtgatttagtttatatgtttttggaagaaaccattaatgtttaatttaatcttaataccaaaacgaccagattgaagtttgacttttattgtcacaaatcagcaggcacaagaactgtccatgaagatgggaaatagccttaaggctgatgcagtttacttacaagtttagaaaccagaatgctttgtttttaccagattcaccattagaggttgatggggcaactgcagcccatgacacaagatctcattgttcctcgatgtagag

SEQ ID NO: 379 - DNA, 257 bp, Homo sapiens
gatctgtacctaagctcagtatgcaaaagcccaaactagtgcagaaactgtaaactgtgcctttcaaagaactggccctaggtgaacaggaaaacaatgaagtttgcatgactaaattgcagaagcataattttatttttttggagcactttttcagcaatattagcggctgaggggctcaggatctattttaatatttcaattattcttccatttcatatagtgatcacaagcagggggttctgcaattccgttca

SEQ ID NO: 380 - DNA, 527 bp, Homo sapiens
gggagatttaaattgcgctaacagtgttgcgcaaaaatgagttcatgccatttaacatattgtattttaattattaactgtattaatttactatgaaatggacatccttttaactaaaatggaattgaacattgcagttttcaaatatttttccttgttgggtctggaaaaggaattctactttgatctgcatagaaaattttgatacaattttttgaaagttcttaggtgaaacatttacccattaaaaaggaagcagaaatactgagacatgaaaggcattatcaactaactctagactctagaacccattctagcatatctcacgtgcaatttttaaaaataagttaataattcatctcatatcaacaaaagcctttgaaacatgggttttcactagatatcacctagtgctaagataaaaaccaaaacaatatcagaattacatttatgctctaaatttgtagttgtccattgttgtgcttagtaaatgtgtgtcattaatgctgtattctcctagctattat

SEQ ID NO: 381 - DNA, 235 bp, Homo sapiens
gtgcatgtgaccacaaatgcttgcttggacttgcccatctagcactttggaaatcagtatttaaatgccaaataatcttccaggtagtgctgcttctgaagttatctcttaatcctcttaagtaatttggtgtctgtccagaaaaagtcgatttatgtgtattaattggccatcatgatgttatcatatcttattcccttttatgctatgatttattctatcttttgtatttcag

SEQ ID NO: 382 - DNA, 444 bp, Homo sapiens
gatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaa

SEQ ID NO: 383 - DNA, 166 bp, Homo sapiens; identical to SEQ ID NO: 358

SEQ ID NO: 384 - DNA, 525 bp, Homo sapiens
tcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgctt

SEQ ID NO: 385 - DNA, 320 bp, Homo sapiens
  modified_base (214)..(215): a, c, t, or g
aaggttagtgactctgcaggacttcacagagagagctgtgcccatcattcagtccaagtgctttctctgcccagacagcacagaactccagccccgctacttacatggatcatcgagtttccacctaaaatatgattctatttattttgagtcactgttaccaaattagaactaaaacaaagttacataaaaagttattgtgactccacttaannttagtgacgtatttttgtatatataggccaacctataccacatccaaaattatgtatctattacagcccctagaagctttataaatacagtgtgtcttcttttat

SEQ ID NO: 386 - DNA, 526 bp, Homo sapiens
gacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcacgctgatccccatcgctgtgggtggtgccctggcggggctggtcctcatcgtcctcatcgcctacctcgtcggcaggaagaggagtcacgcaggctaccagactatctagcctggtgcacgcaggcacagc

SEQ ID NO: 387 - DNA, 493 bp, Homo sapiens
ggtgaagccagtgtactttctcagtgagttctctgcataaagactaatcagtgggaccaggtaaaaaggtcatataatacattgtggagattgcttacttaatacttctgaaaaatggagtaagggagaaactgtaatgttgcaatatgaacctcccattgggccttccatagggaaagctgtgactactctgaaatggaacctagcattatatccttgtagggtagattataaatcatttccagttcatttctcttagaggtgattacctctagccatcagccttactccatcccatgtttggtatgcaatttgagccacaaggctcgtatcgccaacagctatatacattttgttccatttttctgtcttacagagccatgataaaactgtggttagtgagttaaaattcctggagtaactactgtttttctcctttgaaacttaggtttctaaagttgcacctaaggaatctgtcacattttctgttgaa

SEQ ID NO: 388 - DNA, 273 bp, Homo sapiens
  modified_base (68)..(68): a, c, t, or g
aggaacagacctttggcttggagttgatgcccttggactgaatatttatgagaaagatgataagttanccccaaagattggctttccttggagtgaaatcaggaacatctctttcaatgacaaaaagtttgtcattaaacccatcgacaagaaggcacctgactttgtgttttatgccccacgtctgagaatcaacaagcggatcctgcagctctgcatgggcaaccatgagttgtatatgcgccgcaggaagcctgacaccatcgaggtgca

SEQ ID NO: 389 - DNA, 515 bp, Homo sapiens
gatagggttactacttgagttgctatggctccagctgaaagaaagcccgtgcagtcatatcacgcgtaaacatttgctttatgctaaaaatatggtggacctggcattacagctattacaaatctcctaagatgtctcgggtagtgtattagttacttttcatactgctatgaagaaatactggaaactgggtaatttataaagaaaaagaggtttaatgtactcacagttccacaaggctggagaggcctcagaatcatggtggaaggcaaagaaggagcaaaaaggtatgtcttccatggcagcaggcaagagagcacgtgcagggaaactgccctttataaaaccatcagatttagtgagatgtattcactatcacgagaacagtatgggaaaaacctgcccccatgattcgattacctcctaccgggtccctcccacgacacatggggattatgggaactacaattcaagatgaaatttgggtggggacgcagccaaaccatatcgggtag

SEQ ID NO: 390 - DNA, 213 bp, Homo sapiens
tcatctttttcacccatcagaatttgtctcaggattacttggtttttctcagtcctcaagcgagaacttgcttttctttgttaatgtgactttcattactgagtacccacgtatttggagtatgagaaggtgggttatttctcatactctgtccctctctttttcattgaatgtaagagtacattttaatgttgcttcagtgattgtataatg

SEQ ID NO: 391 - DNA, 447 bp, Homo sapiens
gaattagccaaaacacttaggaagaaatcacttaaatacctctggcttagaaattttttcatgcacactgttggaatgtatgctaattgaacatgcaattggggaagaaaaaatgtagaatgatttttgctatttctagtagaaagaaaatgtctgttttccaaagataatgttatacatcctattttgtaatttttttgaaaaaagttcaatgttcagttttccttagtttttaccttgttttctctataggtcatgatttctgtgaagcaaaaagatgccttttaccatgaattcttgagtttacatcaataatattgtatattaaggggatcagaagtaggaaggaaaaaataagagatagcagaggaaaaagaaaaacatttcctcttataacttctgaagtaatttgtaaaaaagatttgtagagtcaatcatgtgtttaaa

SEQ ID NO: 392 - DNA, 489 bp, Homo sapiens
ttcctaggtttcactctttttagtgtactagatccagaaacttagtgtaatgccctgctttatatttctttgacttaacattggtttcagaaagaatcttagctacctagaatttacagtctctgtttcatggcaacactggataatggctttgtgaaatttaaaaaatttttgtagcgactgtaaacagaaatgccaaattgatggttaattgttgctgcttcaaaaataagtataaaattaatatgtaaggaagcccattctttcatgttaaatacttggggtgggaggggagaaagggaaccttttcttaaaatgaaaataattactgctattttaaaatttcttgatcattgaatgtgagacccttctaacatgatttgagaagctgtacaagtataggcagagttattttcctgtttacattttttttttgttttggggaaaaaattggtaggtgtctaattactgtttacttcattgttatat

SEQ ID NO: 393 - DNA, 268 bp, Homo sapiens
gaattgtgacttgcaccttttgttcaaagtatttcctttaggcattgtaattgtgaacagctcttacttgtgccagtgacagatgcagtggtctcctttccccagttgaagcagtgcatacgcagtagctattatttgtgttatctttatttctcttcattgttagaaaccaaagtcttctctgctggctggggctgagagagggtctgggttatctccttctgatcttcaaaacaagagagagaccttgaatacactgactcttcca

SEQ ID NO: 394 - DNA, 413 bp, Homo sapiens
  modified_base (39)..(39): a, c, t, or g
  modified_base (368)..(368): a, c, t, or g
gttttcaactcaggaactatggcaaggaactttccccanatcaaattctattaacgctgagatacaagtcatccatgcacagccactatcataccctttattctcactgaaaggcagaactcagaacctgttattttatgtctgtaatcatgtactttggcatcttttggaggaaaggggcaggataactcactggaatgtacagtattttgctagtgcatttcaaggaatggaatcttctccagtatgaaattaccagatataaaataatgtaatgatgctgaggatataagcttttagaaggtaatttgatggtatttctttctcgaatgaaaagctgctggtttaccctcaaccctattcattancattaccatgagtgaatttatatctaattatttccacttgccctg

SEQ ID NO: 395 - DNA, 499 bp, Homo sapiens
  modified_base (95)..(96): a, c, t, or g
tgaaacaaacagctgacgtcctctctcgatctgcaagcctttcaccaaccaaatagttgcctctctcgtcaccaaactggaacctcacaccagcnngcaaaggaaggaagaaaggttttagagctgtgtgttctttctctggctttgattcttctttgagttctcttacttgccacgtacaggaccattatttatgagtgaaaagttgtagcacattccttttgcaggtctgagctaagcccctgaaagcagggtaatgctcataaaaggactgttcccgcggccccaaggtgcctgttgttcacacttaagggaagtttataaagctactggccccagatgctcagggtaaggagcaccaaagctgaggctggctcagagatctccagagaagctgcagcctgccctggccctggctctggccctggcccacattgcacatggaaacccaaaggcatatatctgcgtatgtgtggtacttagtcacatctttgtcaac

SEQ ID NO: 396 - DNA, 185 bp, Homo sapiens
aggagattttccttcgggagttgatctctaatgcttctgatgccttggacaagattcgctatgagagcctgacagacccttcgaagttggacagtggtaaagagctgaaaattgacatcatccccaaccctcaggaacgtaccctgactttggtagacacaggcattggcatgaccaaagctgat

SEQ ID NO: 397 - DNA, 487 bp, Homo sapiens
taatcttgctcattaaccttactcctttgagaattctttaacaatatttaaaattggtaacaaaaatagtttagccataattgtttagccatgtgagtttcaggttggtacacgttcagacagaactgctgtatcacattccaattttgaatagccagtgagcaatcaagtgtagagaaatgataaatggcctaagaaggcatacagtggcataaacgatgctcttcctagtagcttaataggccacaagctagtttctgttgccctctgaaataaaatatgctttaaaaatgtagggaccagtgcttagaaaagcaaaaactaggtgtgtcattgaaataataggcataaaaattaaatgttacataagacccctatttggaaaaagggtccttttaaaaactgaatttgtactaaatcagatttgccatgtccagtacagaataatttgtacttagtatttgcagcagggtttgtctttgtgaat